(19) United States
(12) Patent Application Publication
(10) Pub. No.: US 2025/0340604 A1
(43) Pub. Date: Nov. 6, 2025

(54) BLOOD-BRAIN BARRIER TRANSLOCATING PEPTIDES AND RELATED MOLECULES AND METHODS OF USE THEREOF

(71) Applicants: Enkefalos Biosciences, Inc., San Diego, CA (US); Enkefalos Biosciences, LLC, San Diego, CA (US)

(72) Inventors: Duncan MCGREGOR, San Diego, CA (US); William ELDRIDGE, Cambridge (GB); Vaughn SMIDER, San Diego, CA (US); Charles MELANCON, San Diego, CA (US)

(73) Assignees: Enkefalos Biosciences, Inc., San Diego, CA (US); Enkefalos Biosciences, LLC, San Diego, CA (US)

(21) Appl. No.: 18/865,655

(22) PCT Filed: May 15, 2023
(86) PCT No.: PCT/US2023/067027

§ 371 Date: Nov. 13, 2024

Related U.S. Application Data
(60) Provisional application No. 63/342,622, filed on May 16, 2022.

(51) Int. Cl. C07K 14/415; A61K 38/00; A61K 39/00; C07K 14/48; C07K 16/24; C07K 16/32; C12N 9/24

(52) U.S. Cl. CPC C07K 14/415; C07K 14/48; C07K 16/241; C07K 16/32; C12N 9/2402; C12Y 302/01045; A61K 38/00; A61K 2039/505; C07K 2317/92; C07K 2319/01

(57) ABSTRACT

Provided are peptides able to bind a receptor that mediates receptor-mediating transcytosis (RMT) across the blood-brain barrier (BBB), as well as binding molecules that incorporate the peptides. Also provided are conjugates, including fusion proteins, composed of the peptides or binding molecules and a therapeutic or diagnostic agent. In some embodiments, the conjugates are able to pass through the blood-brain barrier after being parenterally administered to allow for function of the therapeutic or diagnostic agent in the central nervous system. Also provided are methods of making and using the provided peptides and molecules.

This application claims priority from U.S. provisional application No. 63/342,622, filed May 16, 2022, entitled “BLOOD-BRAIN BARRIER TRANSLOCATING PEPTIDES AND RELATED MOLECULES AND METHODS OF USE THEREOF,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 166302000240SeqList.xml, created May 15, 2023, which is 253,440 kilobytes in size. The information in the electronic format of the Sequence Listing is incorporated by reference in its entirety.

### FIELD

The present disclosure provides peptides able to bind a receptor that mediates receptor-mediating transcytosis (RMT) across the blood-brain barrier (BBB), as well as binding molecules that incorporate the peptides. The present disclosure also provides conjugates, including fusion proteins, composed of the peptides or binding molecules and a therapeutic or diagnostic agent. In some embodiments, the conjugates are able to pass through the blood-brain barrier after being parenterally administered to allow for function of the therapeutic or diagnostic agent in the central nervous system. The present disclosure also provides methods of making and using the provided peptides and molecules.

### BACKGROUND

The blood-brain barrier (BBB) performs a neuroprotective function by tightly controlling access to the brain; consequently it also impedes access of pharmacological agents to cerebral tissues, necessitating the use of vectors for their transit. BBB permeability is frequently a rate-limiting factor for the penetration of drugs or peptides into the central nervous system (CNS) (see Pardridge, W. M. J. Neurovirol. 5: 556-569 (1999); Bickel, U., Yoshikawa, T. & Pardridge, W. M. Adv. Drug Deliv. Rev. 46: 247-279 (2001). The brain is shielded against potentially toxic substances by the BBB, which is formed by brain capillary endothelial cells that are closely sealed by tight junctions. In addition, brain capillaries possess few fenestrae and few endocytic vesicles, compared to the capillaries of other organs (see Pardridge, W. M. J. Neurovirol. 5: 556-569 (1999)). There is little transit across the BBB of large, hydrophilic molecules aside from some specific proteins such as transferrin, lactoferrin and low-density lipoproteins, which are taken up by receptor-mediated endocytosis (see Pardridge, W. M. J. Neurovirol. 5: 556-569 (1999); Tsuji, A. & Tamai, I. Adv. Drug Deliv. Rev. 36: 277-290 (1999); Kusuhara, H. & Sugiyama, Y. Drug Discov. Today 6:150-156 (2001); Dehouck, B. et al. J. Cell. Biol. 138: 877-889 (1997); and Fillebeen, C. et al. J. Biol. Chem. 274: 7011-7017 (1999). Improved molecules are therefore needed, for example, to allow certain agents, such as therapeutic and diagnostic agents, to transit the BBB. Provided are molecules that meet such needs.

### SUMMARY

Provided herein is a modified cyclotide comprising i) a peptide that binds to a blood-brain barrier trancytosis receptor (BBB-R) selected from the group consisting of transferrin receptor (TrfR), insulin-like growth factor type 1 receptor (IGFR), Erb-B2 Receptor Tyrosine Kinase 3 (ErbB3), leptin receptor (ObR), low-density lipoprotein receptor-related protein 1 (LRP-1), and receptor for advanced glycation-end products (RAGE), wherein said peptide has an amino acid sequence of 2 to 50 amino acid residues; and ii) a cyclotide scaffold comprising the peptide of i), wherein the modified cyclotide comprises the structure (I):

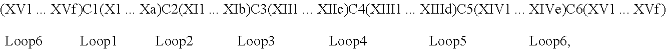

wherein C1 to C6 are cysteine residues; wherein each of C1 and C4, C2 and C5, and C3 and C6 are connected by a disulfide bond to form a cysteine knot; wherein each X represents an amino acid residue in a loop, wherein said amino acid residues are the same or different; wherein d is about 1-2; wherein at least one loop from loops 1, 2, 3, 5 or 6 has an amino acid sequence comprising the sequence of said peptide of clause i), wherein any loop comprising said sequence of said peptide of clause i) comprises 2 to about 30 amino acids, and wherein for any of loops 1, 2, 3, 5, or 6 that do not contain said sequence of said peptide of clause i), a, b, c, e, and f, are the same or different, and are each any number from 3-10, and b, c, e, and f are each any number from 1 to 20.

In some of any of the provided embodiments, the BBB-R is human. In some of any of the provided embodiments, the cyclotide scaffold is selected from a plant cyclotide. In some of any of the provided embodiments, the cyclotide scaffold is of the Momordicae species. In some of any of the provided embodiments, the cyclotide scaffold is a Momordica cochinchinensis trypsin inhibitor. In some of any of the provided embodiments, the Momordica cochinchinensis trypsin inhibitor is MCoTI-I set forth in SEQ ID NO: 1, MCoTI-II set forth in SEQ ID NO: 2 or MCoTI-III set forth in SEQ ID NO: 3. In some of any of the provided embodiments, the sequence of said peptide replaces or substitutes one or more amino acids of one of the one or more loops of the cyclotide scaffold.

Provided herein is a modified cyclotide comprising a peptide that binds to a blood-brain barrier trancytosis receptor (BBB-R), wherein: the peptide is inserted into or replaces one or more amino acids of at least one loop of the cyclotide scaffold set forth in SEQ ID NO:1, SEQ ID NO:2 or SEQ ID NO: 3, and wherein the peptide is about 2 to 50 amino acid residues; and the BBB-R is selected from the group consisting of transferrin receptor (TrfR), insulin-like growth factor type 1 receptor (IGFR), Erb-B2 Receptor Tyrosine Kinase 3 (ErbB3), leptin receptor (ObR), low-density lipoprotein receptor-related protein 1 (LRP-1).

In some of any of the provided embodiments, the cyclotide scaffold is set forth in SEQ ID NO:2. In some of any of the provided embodiments, the at least one loop is loop 1, loop 5 or loop 6, or is a combination thereof. In some of any of the provided embodiments, the at least one loop is loop 1. In some of any of the provided embodiments, the peptide is inserted into and replaces amino acids in only one loop of the cyclotide scaffold. In some of any of the provided embodiments, the only loop is loop 1. In some of any of the provided embodiments, the cyclotide scaffold is set forth in SEQ ID NO:2 and the peptide replaces loop 1 amino acids between cysteine 4 and cysteine 11 of SEQ ID NO:2. In some of any of the provided embodiments, all amino acids of the at least one loop are replaced by the peptide.

Provided herein is a modified cyclotide comprising a peptide inserted into loop 1 to replace all amino acids between cysteine 4 and cysteine 11 of SEQ ID NO:2, wherein the peptide is 2 to 50 amino acid residues and binds to a blood-brain barrier trancytosis receptor (BBB-R) selected from the group consisting of transferrin receptor (TrfR), insulin-like growth factor type 1 receptor (IGFR), Erb-B2 Receptor Tyrosine Kinase 3 (ErbB3), leptin receptor (ObR), low-density lipoprotein receptor-related protein 1 (LRP-1).

In some of any of the provided embodiments, the peptide is 2 to 40 amino acids, 2 to 30 amino acids, 2 to 25 amino acids, 2 to 20 amino acids, 2 to 15 amino acids, 2 to 10 amino acids, 2 to 5 amino acids, 5 to 50 amino acids, 5 to 40 amino acids, 5 to 30 amino acids, 5 to 25 amino acids, 5 to 20 amino acids, 5 to 15 amino acids, 5 to 10 amino acids, 10 to 50 amino acids, 10 to 40 amino acids, 10 to 30 amino acids, 10 to 25 amino acids, 10 to 15 amino acids, 15 to 50 amino acids, 15 to 40 amino acids, 15 to 30 amino acids, 15 to 25 amino acids, 15 to 20 amino acids, 20 to 50 amino acids, 20 to 40 amino acids, 20 to 30 amino acids, 20 to 25 amino acids, 25 to 50 amino acids, 25 to 40 amino acids, 25 to 30 amino acids, 30 to 50 amino acids, 30 to 40 amino acids, or 40 to 50 amino acids. In some of any of the provided embodiments, the peptide is 2 to 30 amino acids, such as 2 to 24 amino acids, 2 to 18 amino acids, 2 to 12 amino acids, 2 to 6 amino acids, 6 to 30 amino acids, 6 to 24 amino acids, 6 to 18 amino acids, 6 to 12 amino acids, 12 to 30 amino acids, 12 to 24 amino acids, 12 to 18 amino acids, 18 to 30 amino acids, 18 to 24 amino acids or 24 to 30 amino acids. In some of any of the provided embodiments, the peptide is 14 to 20 amino acids. In some of any of the provided embodiments, the peptide is 10 amino acids. In some of any of the provided embodiments, the peptide is 12 amino acids.

In some of any of the provided embodiments, the BBB-R is expressed on brain endothelial cells. In some of any of the provided embodiments, the peptide has blood-brain barrier translocation activity. In some of any of the provided embodiments, the BBB-R is the transferrin receptor.

In some of any of the provided embodiments, the peptide comprises the sequence set forth in any one of SEQ ID NOS: 26-34 and 49-54. In some embodiments, the peptide is set forth in any one of SEQ ID NOS: 26-34 and 49-54. In some of any of the provided embodiments, the peptide has the consensus motif set forth as xxxxxHxxSWGx (SEQ ID NOL:177). In some of any of the provided embodiments, the modified cyclotide comprises the sequence set forth in any one of SEQ ID NOS: 72-80 and 95-100. In some embodiments, the modified cyclotide is set forth in any one of SEQ ID NOS: 72-80 and 95-100. In some of any of the provided embodiments, the peptide comprises the sequence forth in SEQ ID NO:26. In some embodiments, the peptide is set forth in SEQ ID NO:26. In some of any of the provided embodiments, the modified cyclotide comprises the sequence set forth in SEQ ID NO:72. In some embodiments, the modified cyclotide is set forth in SEQ ID NO:72. In some embodiments, the peptide comprises the sequence forth in SEQ ID NO:49. In some embodiments, the peptide is set forth in SEQ ID NO:49. In some embodiments, the modified cyclotide comprises the sequence set forth in SEQ ID NO:95. In some embodiments, the modified cyclotide is set forth in SEQ ID NO:95.

In some of any of the provided embodiments, the peptide comprises a sequence with 1, 2, 3 or 4 amino acid substitution(s) compared to the sequence set forth in any one of SEQ ID NOS: 26-34 and 49-54. In some embodiments, the amino acid substitution(s) is substitution of an amino acid to another amino acid selected from histidine or an alanine. In some embodiments, the peptide comprises a sequence with 1, 2, 3 or 4 amino acid substitution(s) compared to the sequence set forth in of SEQ ID NO: 26. In some of any of the provided embodiments, the peptide comprises the sequence set forth in any one of SEQ ID NOS: 55 and 117-128. In some of any of the provided embodiments, the peptide is set forth in any one of SEQ ID NOS: 55 and 117-128. In some embodiments, the modified cyclotide comprises the sequence set forth in any one of SEQ ID NOS: 101 and 105-116. In some embodiments, the modified cyclotide is set forth in any one of SEQ ID NOS: 101 and 105-116. In some of any of the provided embodiments, the peptide comprises the sequence set forth in SEQ ID NO:55. In some embodiments, the peptide is set forth in SEQ ID NO:55. In some embodiments, the modified cyclotide comprises the sequence set forth in SEQ ID NO:101. In some embodiments, the modified cyclotide is set forth in SEQ ID NO:101.

In some of any of the provided embodiments, the BBB-R is the leptin receptor. In some of any of the provided embodiments, the peptide comprises the sequence set forth in any one of SEQ ID NOS: 10-23. In some of any of the provided embodiments, the peptide is set forth in any one of SEQ ID NOS: 10-23. In some of any of the provided embodiments, the modified cyclotide comprises the sequence set forth in any one of SEQ ID NOS: 56-69. In some embodiments, the modified cyclotide is set forth in any one of SEQ ID NOS: 56-69. In some of any of the provided embodiments, the peptide comprises a sequence with 1, 2, 3 or 4 amino acid substitution(s) compared to the sequence set forth in any one of SEQ ID NOS: 10-23. In some embodiments, the amino acid substitution(s) is substitution of an amino acid to another amino acid selected from histidine or an alanine.

In some of any of the provided embodiments, the BBB-R is ErbB3. In some of any of the provided embodiments, the peptide comprises the sequence set forth in SEQ ID NO:24 or 25. In some of any of the provided embodiments, the peptide is set forth in SEQ ID NO: 24 or 25. In some of any of the provided embodiments, the modified cyclotide comprises the sequence set forth SEQ ID NO: 70 or 71. In some embodiments, the modified cyclotide is set forth in SEQ ID NO: 70 or 71. In some of any of the provided embodiments, the peptide comprises a sequence with 1, 2, 3 or 4 amino acid substitution(s) compared to the sequence set forth in SEQ ID NO: 24 or 25. In some embodiments, the amino acid substitution(s) is substitution of an amino acid to another amino acid selected from histidine or an alanine.

In some of any of the provided embodiments, the BBB-R is insulin-like growth factor type 1 receptor (IGFR). In some of any of the provided embodiments, the peptide comprises the sequence set forth in SEQ ID NO:35 or 36. In some of any of the provided embodiments, the peptide is set forth in SEQ ID NO: 35 or 36. In some of any of the provided embodiments, the peptide comprises the sequence set forth in SEQ ID NO:36. In some embodiments, the peptide is set forth in SEQ ID NO:36. In some of any of the provided embodiments, the modified cyclotide comprises the sequence set forth SEQ ID NO: 81 or 82. In some embodiments, the modified cyclotide is set forth in SEQ ID NO: 81 or 82. In some of any of the provided embodiments, the modified cycltoide comprises the sequence set forth in SEQ ID NO:82. In some embodiments, the modified cyclotide is set forth in SEQ ID NO:82. In some of any of the provided embodiments, the peptide comprises a sequence with 1, 2, 3 or 4 amino acid substitution(s) compared to the sequence set forth in SEQ ID NO: 35 or 36. In some embodiments, the amino acid substitution(s) is substitution of an amino acid to another amino acid selected from histidine or an alanine.

In some of any of the provided embodiments, the BBB-R is RAGE. In some of any of the provided embodiments, the peptide comprises the sequence set forth in SEQ ID NO:37 or 38. In some of any of the provided embodiments, the peptide is set forth in SEQ ID NO: 37 or 38. In some of any of the provided embodiments, the modified cyclotide comprises the sequence set forth SEQ ID NO: 83 or 84. In some embodiments, the modified cyclotide is set forth in SEQ ID NO: 83 or 84. In some of any of the provided embodiments, the peptide comprises a sequence with 1, 2, 3 or 4 amino acid substitution(s) compared to the sequence set forth in SEQ ID NO: 37 or 38. In some embodiments, the amino acid substitution(s) is to a histidine or an alanine.

In some of any of the provided embodiments, the BBB-R is the 1 lipoprotein receptor-related protein 1 (LRP-1). In some of any of the provided embodiments, the peptide comprises the sequence set forth in any one of SEQ ID NOS: 39-48. In some of any of the provided embodiments, the peptide is set forth in any one of SEQ ID NOS: 39-48. In some embodiments, the peptide comprises the sequence set forth in SEQ ID NO:39. In some embodiments, the peptide is set forth in SEQ ID NO:39. In some embodiments, the peptide comprises the sequence set forth in SEQ ID NO:43. In some embodiments, the peptide is set forth in SEQ ID NO:43. In some embodiments, the peptide comprises the sequence set forth in SEQ ID NO:47. In some embodiments, the peptide is set forth in SEQ ID NO:47. In some of any of the provided embodiments, the modified cyclotide comprises the sequence set forth in any one of SEQ ID NOS: 85-94. In some embodiments, the modified cyclotide is set forth in any one of SEQ ID NOS: 85-94. In some embodiments, the modified cyclotide comprises the sequence set forth in SEQ ID NO:85. In some embodiments, the modified cyclotide is set forth in SEQ ID NO:85. In some embodiments, the modified cyclotide comprises the sequence set forth in SEQ ID NO:89. In some embodiments, the modified cyclotide is set forth in SEQ ID NO:89. In some embodiments, the modified cyclotide comprises the sequence set forth in SEQ ID NO:93. In some embodiments, the modified cyclotide is set forth in SEQ ID NO:93. In some of any of the provided embodiments, the peptide comprises a sequence with 1, 2, 3 or 4 amino acid substitution(s) compared to the sequence set forth in any one of SEQ ID NOS: 39-48. In some embodiments, the amino acid substitution(s) is substitution of an amino acid to another amino acid selected from histidine or an alanine.

Provided herein is a peptide comprising the amino acid sequence set forth in any of SEQ ID NOs: 10-55 or 117-128, wherein the peptide is 6-50 amino acids in length and binds to a receptor involved in blood-brain barrier transcytosis (BBB-R).

In some of any of the provided embodiments, the peptide has blood-brain barrier translocation activity. In some of any of the provided embodiments, the peptide is 2 to 40 amino acids, 2 to 30 amino acids, 2 to 25 amino acids, 2 to 20 amino acids, 2 to 15 amino acids, 2 to 10 amino acids, 2 to 5 amino acids, 5 to 50 amino acids, 5 to 40 amino acids, 5 to 30 amino acids, 5 to 25 amino acids, 5 to 20 amino acids, 5 to 15 amino acids, 5 to 10 amino acids, 10 to 50 amino acids, 10 to 40 amino acids, 10 to 30 amino acids, 10 to 25 amino acids, 10 to 15 amino acids, 15 to 50 amino acids, 15 to 40 amino acids, 15 to 30 amino acids, 15 to 25 amino acids, 15 to 20 amino acids, 20 to 50 amino acids, 20 to 40 amino acids, 20 to 30 amino acids, 20 to 25 amino acids, 25 to 50 amino acids, 25 to 40 amino acids, 25 to 30 amino acids, 30 to 50 amino acids, 30 to 40 amino acids, or 40 to 50 amino acids. In some of any of the provided embodiments, the peptide is 2 to 30 amino acids, such as 2 to 24 amino acids, 2 to 18 amino acids, 2 to 12 amino acids, 2 to 6 amino acids, 6 to 30 amino acids, 6 to 24 amino acids, 6 to 18 amino acids, 6 to 12 amino acids, 12 to 30 amino acids, 12 to 24 amino acids, 12 to 18 amino acids, 18 to 30 amino acids, 18 to 24 amino acids or 24 to 30 amino acids. In some of any of the provided embodiments, the peptide is 14 to 20 amino acids. In some of any of the provided embodiments, the peptide is 10 amino acids. In some of any of the provided embodiments, the peptide is 12 amino acids.

Provided herein is a peptide consisting of the sequence set forth in any one of SEQ ID NOs: SEQ ID NOs: 10-55 or 117-128. In some of any of the provided embodiments, the peptide binds to a receptor involved in blood-brain barrier transcytosis.

Provided herein is a peptide set forth by the sequence of any of SEQ ID NOS: 26-34 and 49-55 and 117-128, wherein the peptide binds the transferrin receptor. In some of any of the provided embodiments, the peptide is set forth in SEQ ID NO:26. In some of any of the provided embodiments, the peptide is set forth in SEQ ID NO: 49. In some of any of the provided embodiments, the peptide is set forth in SEQ ID NO: 55.

Provided herein is a peptide set forth by the sequence of any of SEQ ID NOS: 10-23, wherein the peptide binds the leptin receptor. Provided herein is a peptide set forth by the sequence of any of SEQ ID NOS: 24 or 25, wherein the peptide binds ErbR3.

Provided herein is a peptide set forth by the sequence of any of SEQ ID NOS: 35 or 36, wherein the peptide binds IGFR. In some of any of the provided embodiments, the peptide is set forth in SEQ ID NO: 35. In some of any of the provided embodiments, the peptide is set forth in SEQ ID NO:36.

Provided herein is a peptide set forth by the sequence of SEQ ID NO: 37 or 38, wherein the peptide binds the RAGE.

Provided herein is a peptide set forth by the sequence of any of SEQ ID NOS: 39-48, wherein the peptide binds the lipoprotein receptor-related protein 1 (LRP-1). In some of any of the provided embodiments, the peptide is set forth in SEQ ID NO:39. In some of any of the provided embodiments, the peptide is set forth in SEQ ID NO43. In some of any of the provided embodiments, the peptide is set forth in SEQ ID NO:47.

In some of any of the provided embodiments, the peptide is synthetic. In some of any of the provided embodiments, the peptide is isolated.

Provided here is a binding molecule comprising a binding scaffold and any of the provided peptides. In some of any of the provided embodiments, the binding scaffold is a cyclotide. In some of any of the provided embodiments, the peptide is inserted into or replaces one or more amino acids of a loop of a cyclotide backbone. In some embodiments, the at least one loop is loop 1. In some of any of the provided embodiments, the binding scaffold is set forth in any one of SEQ ID NOS: 1-3. In some of any of the provided embodiments, the binding scaffold is set forth in SEQ ID NO:2.

Provided herein is a nucleic acid molecule encoding any of the provided modified cyclotides or any of the provided binding molecules.

Provided herein is a vector comprising any of the provided nucleic acids. In some of any of the provided embodiments, the vector is an expression vector.

Provided herein is a host cell comprising any of the provided nucleic acid molecules or any of the provided vectors.

Provided herein is a method of producing a modified cyclotide or binding molecule, the method comprising introducing the nucleic acid of claim 87 or the vector of claim 88 or claim 89 into a host cell and culturing the host cell under conditions to express the protein in the cell. In some embodiments, the method further includes purifying the protein from the cell.

Provided herein is a purified binding molecule or modified cyclotide produced by any of the provided methods.

Provided herein is a conjugate comprising any of the provided modified cyclotides, or any of the provided binding molecules, and a biological active agent. In some of any of the provided embodiments, the biologically active agent is a small molecule, a peptide or a protein. In some of any of the provided embodiments, the biologically active agent is a diagnostic agent or a therapeutic agent. In some of any of the provided embodiments, any of the provided conjugates is a fusion protein comprising the modified cyclotide operably linked to a biologically active agent that is a protein or peptide.

Provided herein is a fusion protein comprising any of the provided modified cyclotides or any of the provided binding molecules and a biologically active agent that is a protein or peptide.

In some of any of the provided embodiments, the biologically active agent is an antibody. In some embodiments, the antibody is directed against an antigen selected from the group consisting of human epidermal growth factor receptor 2 (HER2), beta-secretase 1 (BACE1), amyloid beta (Abeta), epidermal growth factor receptor (EGFR), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), caspase 6 and TNF-alpha.

In some of any of the provided embodiments, the antibody is trastuzumab, adalimumab or aducanumab. In some of any of the provided embodiments, the biologically active agent is a growth factor or a hormone. In some of any of the provided embodiments, the biologically active agent is a growth factor and the growth factor is nerve growth factor (NGF) or Granulocyte colony-stimulating factor (GCSF).

In some of any of the provided embodiments, the biologically active agent is an enzyme. In some of any of the provided embodiments, the enzyme is a ceramide degrading enzyme, a lipase, a hydrolase type enzyme or a sulfatase. In some of any of the provided embodiments, the enzyme is a ceramide degrading enzyme and the ceramide degrading enzyme is glucocerebrosidase, galactocerebrosidase or alpha galactosidase. In some embodiments, the enzyme is a glucocerebrosidase that has a sequence of amino acids that is at least 95% identical to the sequence set forth in SEQ ID NO:144 or SEQ ID NO:145. In some embodiments, the glucocerebrosidase is a variant that contains 1-5 amino acid substitutions compared to the sequence set forth in SEQ ID NO: 144 or SEQ ID NO:145. Non-limiting mutations include any as described herein. In some embodiments, the enzyme is a glucocerebrosidase that has the sequence of amino acids set forth in SEQ ID NO:144. In some embodiments, the enzyme is a glucocerebrosidase that has the sequence of amino acids set forth in SEQ ID NO: 145. In some of any of the provided embodiments, the enzyme is a lipase or a hydrolase type enzyme and the enzyme is sphinomyelinase, cerliponase or alpha glucosidase.

In some of any of the provided embodiments, the conjugate or fusion protein comprises a single modified cyclotide. In some of any of the provided embodiments, the conjugate or fusion protein comprises 2, 3, or 4 modified cyclotides. In some of any of the provided embodiments, each modified cyclotide is the same. In some of any of the provided embodiments, each modified cyclotide is different.

In some of any of the provided embodiments, the conjugate or fusion protein is monovalent for binding a BBB-R.

In some of any of the provided embodiments, the conjugate is bivalent for binding to a BBB-R. In some of any of the provided embodiments, the conjugate or fusion protein comprises at least two different modified cyclotides that bind to different BBB-R.

In some of any of the provided embodiments, the conjugate or fusion protein is bispecific for binding two different BBB-R. In some embodiments, the conjugate is bispecific for binding two different BBB-R and comprises at least two different modified cyclotides that each bind to a different BBB-R.

In some of any of the provided embodiments, the modified cyclotide is linked to the biologically active agent via a linker. In some embodiments, the linker is at least 10 amino acids in length. In some embodiments, the linker is at least 15 amino acids in length. In some embodiments, the linker is 10 to 20 amino acids in length. In some of any of the provided embodiments, the linker is a flexible peptide linker. In som embodiments, the peptide linker comprises the sequence GGGGS (SEQ ID NO:148), (GGGGS)2 (SEQ ID NO:154) or (GGGGS)3 (SEQ ID NO:154). In some of any of the provided embodiments, the linker is set forth in SEQ ID NO:104. In some of any of the provided embodiments, the linker is a cleavable linker comprising an endosome-specific protease cleavage site. In some of any of the provided embodiments, the endosome-specific protease cleavage site is a cathepsin cleavage site. In some of any of the provided embodiments, the cathepsin cleavage site is a cathepsin B cleavage site. In some of any of the provided embodiments, the linker comprises the sequence set forth in SEQ ID NO:133.

Provided herein is a pharmaceutical composition comprising any of the provided conjugates or fusion proteins and a pharmaceutical carrier.

Provided herein is a method for transporting a biologically active agent across a blood brain barrier of an individual, the method comprising administering any of the provided conjugates or fusion proteins or any of the provided pharmaceutical compositions to an individualin need thereof. In some embodiments, the individual is a mammal. In some embodiments, the individual is a human.

In some of any of the provided embodiments, the individual, such as a mammal (e.g, a human) has a neurological disease. In some of any of the provided embodiments, said neurological disease is selected from the group consisting of Alzheimer's disease, Parkinson's disease, stroke, a brain tumor and a brain metastasis. In some of any of the provided embodiments, said neurological disease is a congenital disease that is selected from the group consisting of Austen Disease, Canavan Disease, Gaucher's Disease, Hunter Syndrome, Hurler-Scheie Syndrome, Jansky Bielschowsky Disease, Krabbe Disease, LCAT Deficiency, Lowe Syndrome, Maroteaux-Lamy Syndrome, Morquio Syndrome A, Morquio Syndrome B, Sanfilippo Syndrome A, Sanfilippo Syndrome B, Sanfilippo Syndrome C, Sanfilippo Syndrome D, Spinal Muscular Atrophy, Tay Sachs Disease, and Walker-Warburg Syndrome.

Provided herein is a method for treating a patient having a neurological disease comprising administering any of the provided conjugates or any of the provided pharmaceutical compositions to said patient. Provided herein is a method for diagnosing a neurological disease in a patient in need thereof comprising administering any of the provided conjugates or fusion proteins or any of the provided pharmaceutical compositions to said patient and wherein said conjugate comprises a radiolabel. In some of any of the provided embodiments, said neurological disease is selected from the group consisting of Alzheimer's disease, Parkinson's disease, stroke, a brain tumor and a brain metastasis. In some of any of the provided embodiments, said neurological disease is a congenital disease that is selected from the group consisting of Austen Disease, Canavan Disease, Gaucher's Disease, Hunter Syndrome, Hurler-Scheie Syndrome, Jansky Bielschowsky Disease, Krabbe Disease, LCAT Deficiency, Lowe Syndrome, Maroteaux-Lamy Syndrome, Morquio Syndrome A, Morquio Syndrome B, Sanfilippo Syndrome A, Sanfilippo Syndrome B, Sanfilippo Syndrome C, Sanfilippo Syndrome D, Spinal Muscular Atrophy, Tay Sachs Disease, and Walker-Warburg Syndrome.

Also provided are any of the compositions provided herein for use in the treatment of a neurological disease. Also provided are use of any of the provided pharmaceutical compositions in the manufacture of a medicament for use in the treatment of a neurological disease. Also provided are any of the provided compositions provided herein for use in the diagnosis of a neurological disease. Also provided herein are use of any of the provided pharmaceutical compositions in the manufacture of a medicament for use in the diagnosis of a neurological condition. In some of any of the provided embodiments, said neurological disease is selected from the group consisting of Alzheimer's disease, Parkinson's disease, stroke, a brain tumor and a brain metastasis. In some of any of the provided embodiments, said neurological disease is a congenital disease that is selected from the group consisting of Austen Disease, Canavan Disease, Gaucher's Disease, Hunter Syndrome, Hurler-Scheie Syndrome, Jansky Bielschowsky Disease, Krabbe Disease, LCAT Deficiency, Lowe Syndrome, Maroteaux-Lamy Syndrome, Morquio Syndrome A, Morquio Syndrome B, Sanfilippo Syndrome A, Sanfilippo Syndrome B, Sanfilippo Syndrome C, Sanfilippo Syndrome D, Spinal Muscular Atrophy, Tay Sachs Disease, and Walker-Warburg Syndrome.

### BRIEF DESCRIPTION OF THE DRAWINGS

FIGS. 1A-1D depict exemplary configurations of provided antibody fusion proteins composed of an antibody and at least one modified cyclotide including: a bivalent molecule (FIG. 1A), a bispecific molecule (FIG. 1B), a monovalent molecule in which a hole heavy chain is modified with the modified cyclotide (FIG. 1C), and a monovalent molecule in which a knob heavy chain is modified with the modified cyclotide (FIG. 1D).

FIG. 2 depicts a flow cytometry-based assay for internalization of fluorescently labeled adalimumab-CYC17 cyclotide fusion and adalimumab by transferrin receptor-expressing human cells.

FIG. 3 depicts ELISA results for binding of different adalimumab-cyclotide fusions for binding to transferrin receptor (TfR) coated on a microwell, in which the cyclotide portion was CYC17 or different mutants thereof containing amino acid substitution at the indicated positions (e.g. 5H, refers to substitution to a histidine (H) at position 5).

FIG. 4 depicts binding of different cyclotide antibody fusion molecules to human transferrin receptor, including molecules set forth in FIGS. 1A-ID. The molecules include monovalent molecules in which the exemplary CYC17 was fused to either the knob or hole chain of a heterodimeric aducanumab (Aduhelm) antibody, a bispecific fusion in which CYC17 (directed to TfR) was fused to the hole and CYC27 (directed to IGF1R) was fused to the knob of a heterodimeric aducanumab (Aduhelm) antibody, and a bivalent adalimumab (Humira) containing two copies of the CYC17 cyclotide.

FIG. 5 depicts expression of cyclotide trastuzumab antibody fusion molecules in supernatant from transfected host cells.

FIG. 6A depicts enzymatic activity of the GlcCSase-CYC17 fusion cyclotide in a glucocerebroside activity assay.

FIG. 6B depicts ELISA results for binding of the GlcCSase-CYC17 fusion cyclotide to the human transferrin receptor (hTfR) coated on a microwell.

FIG. 7 depicts ELISA results for binding of the NGF-CYC17 fusion cyclotide to the human transferrin receptor (hTfR) coated on a microwell.

FIG. 8A depicts ELISA results for binding of exemplary trastuzumab fusion cyclotides to human LPR1 coated on a microwell. The fusion molecules included monovalent molecules in which exemplary cyclotides CYC30, CYC32, or CYC34 were fused to the knob chain of a heterodimeric trastuzumab (Herceptin®) antibody.

FIG. 8B depicts quantification results of in vivo cyclotide-mediated trastuzumab delivery into mouse brains at 24 hours compared to trastuzumab alone. Seven antibody-monovalent cyclotide fusion molecules, targeting IGF1R, LRP1, or RAGE receptors, were administered to mice and quantified from fresh brain homogenates. The fusion molecules included monovalent molecules in which exemplary cyclotides CYC26, CYC27, CYC28, CYC29, CYC30, CYC32, or CYC34 were fused to the knob chain of a heterodimeric trastuzumab (Herceptin®) antibody.

FIG. 9 depicts quantification results of in vivo cyclotide-mediated trastuzumab delivery into mouse brains at 24 hours compared to trastuzumab alone. Two antibody-monovalent cyclotide fusion molecules, targeting human transferrin receptor (hTfR), were administered to mice and quantified from fresh brain homogenates. The fusion molecules include monovalent molecules in which exemplary cyclotides CYC26 or CYC27 were fused to the knob chain of a heterodimeric trastuzumab (Herceptin®) antibody.

### DETAILED DESCRIPTION

Provided herein are peptides that exhibit blood-brain barrier translocation activity (including the ability to mediate transcytosis to facilitate crossing of the BBB), such as via binding to a receptor involved in blood-brain barrier transcytosis. The provided peptides can be inserted into a protein scaffold to provide for a larger binding molecule backbone or framework, which, in some aspects, may improve the stability or half-life of the peptide. In some embodiments, the scaffold is a a cyclotide backbone to provide for a modified cyclotide with improved or enhanced blood-brain barrier translocation activity and/or binding to a receptor involved in blood-brain barrier transcytosis. The provided modified cyclotide molecules are non-naturally occurring and demonstrate particular advantage in crossing the blood-brain barrier. The provided modified scaffolds into which is inserted a provided peptide, such as provided modified cyclotides, can be used as carriers for attachment or linkage to other molecules, such as polypeptides, proteins and small molecules, including therapeutic agents or diagnositic agents, to delivery such other molecules across the blood-brain barrier. The provided modified cyclotides may be used to deliver therapeutic agents and other biological agents across the BBB for the treatment of neurological conditions.

A problem in delivery of certain therapeutic or other biological agents is that it is difficult to deliver biological agents administered parenterally, such as therapeutic agents and diagnostic agents, to the central nervous system (CNS) because passive transfer of substances from the capillaries to the brain is restricted. Unlike the capillaries in other tissues such as muscles, the capillaries that supply the blood to most of the brain tissues differ in that the endothelial cells forming their endothelium are mutually connected by tight intercellular junctions. This system, which restricts exchange of substances between the blood and the tissue fluid of the brain through the endothelium of capillaries in the brain, is called the blood-brain barrier or BBB. The BBB thus imposes a problem in the access of therapeutic or other biological agents to the CNS because they cannot always pass through the BBB. For instance approximately only about 0.2% of an monoclonal antibody dose that is administered intravenously will reach the brain when the BBB is intact.

Development of various methods has been attempted to allow transit of biological agents across the BBB. Such attempts have included, for example, encapsulating agents in liposomes, delivering biological agents directly into the brain, or by conjugating the agent to an antibody molecule that binds to a membrane protein on endothelial cells of the brain capillaries. Although certain of these methods could increase trancytosis efficiency, the existing methods are invasive and/or complex. For instance, while antibodies may confer affinity to receptors involved in blood-brain barrier trancytosis, their larger size can reduce manufacturability of a therapeutic fusion protein, such as due to problems with expression, purification or stability.

The provided embodiments address these problems. The provided embodiments relate to novel peptides that exhibit binding to receptors involved in blood-brain barrier transcytosis. These peptides can be incorporated into a binding molecule scaffold to provide stability to the molecule and confer onto the molecule the ability to bind receptors that mediate RMT across the blood-brain barrier (BBB). In some embodiments, the binding molecule scaffold is a cyclotide, which is a cysteine-knot protein. The ability to incorporate peptides into stable scaffolds, such as cyclotides, provides for a highly developable platform involving small, highly stable peptides that express well as fusion proteins. Moreover, due to the smaller size of the scaffold platform, the provided embodiments also can be used to produce multi-domain molecules with the ability to target more than one transcytosis pathway.

In particular, among the provided binding molecule scaffolds are cyclotides, which is a member of the naturally occurring family of cysteine-knot microproteins found in various plant species. Cysteine-knot microproteins (cyclotides) are small peptides, typically consisting of about 30-40 amino acids, which can be found naturally as cyclic or linear forms, where the cyclic form has no free N- or C-terminal amino or carboxyl end. They have a defined structure based on three intramolecular disulfide bonds and a small triple stranded β-sheet (Craik et al., 2001, Toxicon 39, 43-60). The cyclic proteins exhibit conserved cysteine residues defining a structure referred to herein as a “cysteine knot”. This family includes both naturally occurring cyclic molecules and their linear derivatives as well as linear molecules which have undergone cyclization. These molecules are useful as molecular framework structures having enhanced stability over less structured peptides (Colgrave and Craik, 2004, Biochemistry 43, 5965-5975). However, these molecules are not themselves capable of crossing the blood-brain barrier to any great extent, and cannot be used as neurotherapeutic agents themselves or act as carriers of therapeutic peptide or protein agents.

The main cyclotide features are a remarkable stability due to the cysteine knot, a small size making them readily accessible to chemical synthesis, and an excellent tolerance to sequence variations. Cyclotides therefore appear as appealing leads or scaffolds for peptide drug design. The cyclotide scaffold is found in almost 30 different protein families among which conotoxins, spider toxins, squash inhibitors, agouti-related proteins and plant cyclotides are the most populated families. Cyclotides from plants in the Rubiaceae and Violaceae families are for the most part found to be head-to-tail cyclic peptides. However, within the squash inhibitor family of cyclotides both cyclic and linear cyclotides have been identified from Momordica cochinchinensis: the cyclic trypsin inhibitors (MCoTI)-I and —II and their linear counterpart MCoTI-III. It is now clear that both cyclic and linear variants can exist in different cyclotide families, but the impact of the cyclization is poorly understood. Cyclic peptides were expected to display improved stability, better resistance to proteases, and reduced flexibility when compared to their linear counterparts, hopefully resulting in enhanced biological activities. However, linear cyclotides have the advantage of being able to be more easily linked to other peptides or proteins.

In some embodiments, a cyclotide sequence is characterized as having a cysteine knot backbone moiety, in which the cysteine knot backbone comprises the structure (I).

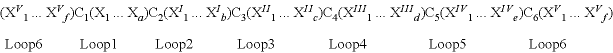

wherein C₁ to C₆ are cysteine residues; wherein each of C₁ and C₄, C₂ and C₅, and C₃ and C₆ are connected by a disulfide bond to form a cysteine knot; wherein each X represents an amino acid residue in a loop, wherein said amino acid residues are the same or different; wherein d is about 1-2; and wherein a, b, c, e, and f, are the same or different, and are each any number from 3-10, and b, c, e, and f are each any number from 1 to 20.

In some embodiments, in addition to stability of the molecule, cyclotides also exhibit features that indicate the molecules have a low immunogenicity risk. Although cyclotides originate as a non-human sequence, they are a heavily disulfide-bonded protein. Evidence indicates that non-human disulfide bonded peptides exhibit no detectable immunogenicity. For instance, Ziconitide is an FDA approved disulfide bonded non-human peptide analogous to cyclotides, and no anti-drug antibodies (ADAs) were detectable, even after repeat dose, in preclinical mouse and rat immunogenicity testing (Skov M. et al. Int. J. Toxicology, 2007 26:411-421).

As cyclotides have not proven to readily cross the BBB in their natural forms, it would be desirable to combine the structural stability of the cyclotide scaffold with the ability to cross the blood-brain barrier, and link other biological activities to the cyclotide molecule. Therefore, identification of cyclotides that are amenable to retaining structure and blood-brain transfer capabilities as both linear or cyclic molecules would be desirable. It would be further desirable to be able use such cyclotides to increase the ability to improve the activity and bioavailability of biological therapeutics in the body.

The provided molecules, including modified cyclotides, address these needs. In some aspects, the provided modified cyclotide molecules are useful as a carrier microprotein peptides capable of crossing the BBB, and can be fused to a desired biological agent, such as a therapeutic molecule, for delivery to the brain.

The embodiments provided herein relate to a cyclotide molecular framework to generate modified cyclotides into which is inserted a peptide sequence to confer enhanced BBB translocation activity relative to the parental cyclotide. In some embodiments, the modified cyclotide is composed of a cysteine-knot backbone that has sufficient disulfide bonds or chemical equivalents thereof to confer a knotted topology on the three-dimensional structure of said cysteine-knot backbone and wherein at least one exposed amino acid residue such as on one or more beta turns and/or within one or more loops, is inserted or substituted (replaced) with a receptor-binding peptide relative to the naturally occurring cyclotide amino acid sequence. In some embodiments, the modified cyclotide has enhanced translocation behaviour compared with the unmodified parental cyclotide. In some embodiments, the modified cyclotide has the desired properties of high enzymatic stability and translocation, such that blood-brain barrier transfer of the modified cyclotide is feasible.

In some embodiments, a modified cyclotide sequence provided herein may be defined as having a cysteine knot backbone moiety and a peptide that is a blood-brain barrier translocation moiety, said modified cyclotide comprising: i) a peptide having said blood-brain barrier translocation activity, wherein said peptide has an amino acid sequence that is about 2 to 50 amino acid residues; and ii) a cysteine knot backbone grafted to said peptide of clause i), wherein said cysteine knot backbone comprises the structure (I):

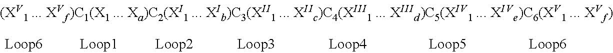

wherein C₁ to C₆ are cysteine residues; wherein each of C₁ and C₄, C₂ and C₅, and C₃ and C₆ are connected by a disulfide bond to form a cysteine knot; wherein each X represents an amino acid residue in a loop, wherein said amino acid residues are the same or different; wherein d is about 1-2; wherein one or more of loops 1, 2, 3, 5 or 6 have an amino acid sequence comprising the sequence of said peptide of clause i), wherein any loop comprising said sequence of said peptide of clause i) comprises 2 to about 50 amino acids, and wherein for any of loops 1, 2, 3, 5, or 6 that do not contain said sequence of said peptide of clause i), a, b, c, e, and f, are the same or different, and are each any number from 3-10, and b, c, e, and f are each any number from 1 to 20.

In some embodiments, the provided modified cyclotide sequence may be either linear or cyclic.

In some embodiments, the cysteine knot backbone is a cyclotide of the Momordicae species including the squash serine protease inhibitor family. Exemplary cyclotides of the Momordicae species include those set forth in SEQ ID NO:1, SEQ ID NO:2 or SEQ ID NO:3. In some embodiments, the peptide is inserted into or replaces one or more amino acids of at least one loop of the cyclotide scaffold set forth in SEQ ID NO:1, SEQ ID NO:2 or SEQ ID NO: 3.

Hence, the cyclotides provided herein are modified cyclotides compared to a natural or wildtype unmodified cyclotide, in which the modified cyclotide has one or more loops inserted or substituted by one or more amino acid sequences, e.g. a peptide of 2 to 50 amino acids. In provided embodiments, the one or more amino acids, e.g. a peptide sequence of 2 to 50 amino acids, that is inserted or substituted into a loop of an unmodified or wildtype cyclotide is a blood-brain barrier translocation moiety that is able to bind to a receptor involved in trancytosis across the blood-brain barrier and/or that mediates blood-brain barrier translocation or permeation of the cyclotide. In particular embodiments, the peptide is one that bind to a transferrin receptor (TrfR), insulin-like growth factor type 1 receptor (IGFR), Erb-B2 Receptor Tyrosine Kinase 3 (ErbB3), leptin receptor (ObR), low-density lipoprotein receptor-related protein 1 (LRP-1), or receptor for advanced glycation end products (RAGE). In aspects of provided embodiments, the modified cyclotides of the invention incorporate sufficient amino acid structure to provide high enzymatic stability and good translocation or permeation behaviour. In some embodiments, the provided modified cyclotides can be used as novel carrier cyclotides to facilitate or mediate translocation of an attached polypeptide, such as a therapeutic or diagnostic agent.

In some embodiments, the peptide-modified cyclotides may be conjugated to another agent or polypeptide, or in certain embodiments one or more modified cyclotides are incorporated into another polypeptide to form a synthetic chimeric (or fusion) polypeptide/protein. Suitably, the peptide of the modified cyclotides may be comprised within a loop region of a scaffold polypeptide. The resultant modified scaffold can then itself be conjugated or comprised within a larger molecule. Suitably cyclotides into which is inserted a receptor-binding peptide as provided herein may be enriched for cysteine residues. In particular embodiments, the provided modified cyclotides exhibit the ability to traverse the blood-brain barrier, suitably rendering the modified cyclotide or any molecule comprising or conjugated thereto available to the brain.

The provided binding molecules, such as modified cyclotides, may be used to deliver a biologically active agent, such as a peptide, polypeptide, protein or small molecule, across the blood-brain barrier. In some embodiments, the biologically active agent is a therapeutic agent, such as a therapeutic agent with therapeutic agonist or antagonist activity. In some embodiments, the biologically active agent is a diagnostic agent.

In some embodiments, a provided modified cyclotide, such as a linear or cyclic cyclotide, is operably linked to the N- or C-terminus of a biologically active agent, such as a peptide or protein moiety. In some embodiments, the linkage may be direct or indirect via a peptide linker. In some embodiments, the linkage provides a conjugate, e.g. fusion protein, of the modified cyclotide and the biologically active agent. Also provided herein is a conjugate, e.g. a fusion protein, comprising a provided modified cyclotide operably linked to the N- or C-terminus of a biologically active agent, such as a peptide or protein moiety.

In some embodiments, the biologically active agent is a therapeutic agent, such as an agent that is agonist or an antagonist. The linkage or fusion of the biologically active agent with a provided modified cyclotide allows the translocation across the BBB and entry of the biologically active agent, such as a peptide or protein moiety. In provided aspects, such linkage allows the translocation across the BBB, and entry of a pharmacologically relevant dose of the biologically active agent, such as a peptide or protein moiety.

In some embodiments, also provided herein are bispecific molecules that incorporate more than one receptor-binding molecule to thereby target more than one transcytosis pathway. For instance, in some embodiments, at least two different modified cyclotides can be linked to a biologically active agent to provide a multidomain protein that may further improve transcytosis capacity of the linked biological agent across the BBB. In some embodiments, 1, 2, 3 or 4 different modified cyclotides each modified with a different receptor-binding peptide may be linked or conjugated to a biological agent to permit engagement of one or multiple BBB transporter pathways.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. RECEPTOR-BINDING PEPTIDES AND MODIFIED CYCLOTIDES

Provided herein are peptides of 2-50 amino acids in length that bind a receptor involved in blood-brain barrier transcytosis. In some embodiments, provided herein are peptides of 6-50 amino acids in length that bind a receptor involved in blood-brain barrier transcytosis. In some aspects, the provided peptides exhibit blood-brain barrier translocation activity. Also provided herein are binding molecules that incorporate any of the provided peptides into a binding molecule scaffold. In some embodiments, the binding molecule scaffold is a small cysteine-knot protein. In some embodiments, the cysteine-knot protein is a cyclotide. Hence, also provided herein are cyclotides that are modified compared to a parental or native cyclotide backbone (also called a cysteine knot backbone), in which the provided modified cyclotides exhibit improved or enhanced ability to bind to a receptor involved in transcytosis across the blood-brain barrier (BBB; hereinafter “BBB trancytosis receptor”) and/or exhibit improved or enhanced blood-brain barrier translocation characteristics.

### A. Receptor-Binding Peptides and Methods of Selection

Provided herein are isolated or synthetic peptides that bind to a BBB transcytosis receptor (also referred to herein as BBB-R). In some embodiments, the peptides are or may be inserted into the backbone of a binding molecule scaffold, such as a cysteine-knot scaffold, for example a cyclotide. Exemplary modified cyclotides containing a receptor-binding peptide are described in subsection I.B.

In some embodiments, the BBB-R is expressed on brain endothelial cells. In some embodiments, the BBB-R is selected from the transferrin receptor (TrfR); the lactoferrin receptor (LtfR); the leptin receptor (LEP-R, or also known as OB-R); the receptor tyrosine-protein kinase (ErbB3), a insulin receptor, such as Insulin Receptor A (IRA), IGF-1 Receptor (IGF-1R), IGF-II Receptor (IGF-IIR), RXFP1, RXFP2, RXFP3 and RXFP4; low-density lipoprotein receptor-related protein 1 (LRP-1), the receptor for advanced glycation end products (RAGE); heparin binding EGF like growth factor receptor (HB EGFR); the intercellular adhesion molecule 1 (ICAM1), intercellular adhesion molecule 2 (ICAM2) or neural cell adhesion molecule (NCAM); or any other receptor that has receptor-mediated transcytosis activity in brain endothelial cells.

In some embodiments, the BBB-R is a mammalian receptor. In some embodiments, the BBB-R is murine. In some embodiments, the BBB-R is human.

In some embodiments, the BBB-R is selected from the human transferrin receptor (human TrfR); the human lactoferrin receptor (human LtFR); the human leptin receptor (human Ob-R); the human ErbB3 receptor, a human insulin receptor, such as Insulin Receptor A (IRA), IGF-1 Receptor (IGF-1R), IGF-II Receptor (IGF-IIR), RXFP1, RXFP2, RXFP3 and RXFP4; the human low-density lipoprotein receptor-related protein 1 (human LRP-1); the human receptor for advanced glycation end products (human RAGE); human heparin binding EGF like growth factor receptor (human HB EGFR); the human intercellular adhesion molecule 1 (human ICAM1), intercellular adhesion molecule 2 (human ICAM2) or neural cell adhesion molecule (human NCAM).

In some embodiments, the BBB-R is Erb-B2 Receptor Tyrosine Kinase 3 (ErbB3), such as human ErbB3. In some embodiments, the BBB-R is TrfR, such as human TrfR. In some embodiments, the BBB-R is leptin receptor (ObR), such as human ObR. In some embodiments, the BBB-R is insulin-like growth factor type 1 receptor (IGFR), such as human IgFR. In some embodiments, the BBB-R is low-density lipoprotein-related protein 1 (LRP-1), such as human LRP-1. In some embodiments, the BBB-R is receptor for advanced glycation end products (RAGE), such as human RAGE.

In some embodiments, the peptides are synthetic peptides. In some embodiments, the peptides are isolated peptides.

In some embodiments, the peptide has an amino acid sequence that is about 2 to 50 amino acid residues in length. In some embodiments, the peptide is 2 to 50 amino acid residues. In some embodiments, the peptide is 2 to 40 amino acids, 2 to 30 amino acids, 2 to 25 amino acids, 2 to 20 amino acids, 2 to 15 amino acids, 2 to 10 amino acids, 2 to 5 amino acids, 5 to 50 amino acids, 5 to 40 amino acids, 5 to 30 amino acids, 5 to 25 amino acids, 5 to 20 amino acids, 5 to 15 amino acids, 5 to 10 amino acids, 10 to 50 amino acids, 10 to 40 amino acids, 10 to 30 amino acids, 10 to 25 amino acids, 10 to 15 amino acids, 15 to 50 amino acids, 15 to 40 amino acids, 15 to 30 amino acids, 15 to 25 amino acids, 15 to 20 amino acids, 20 to 50 amino acids, 20 to 40 amino acids, 20 to 30 amino acids, 20 to 25 amino acids, 25 to 50 amino acids, 25 to 40 aminio acids, 25 to 30 amino acids, 30 to 50 amino acids, 30 to 40 amino acids, or 40 to 50 amino acids. In some embodiments, the peptide is 2 to 30 amino acids, such as 2 to 24 amino acids, 2 to 18 amino acids, 2 to 12 amino acids, 2 to 6 amino acids, 6 to 30 amino acids, 6 to 24 amino acids, 6 to 18 amino acids, 6 to 12 amino acids, 12 to 30 amino acids, 12 to 24 amino acids, 12 to 18 amino acids, 18 to 30 amino acids, 18 to 24 amino acids or 24 to 30 amino acids.

In some embodiments, the peptide is 10 to 25 amino acids. In some embodiments, the peptide is 10 amino acids. In some embodiments, the peptide is 11 amino acids. In some embodiments, the peptide is 12 amino acids. In some embodiments, the peptide is 13 amino acids. In some embodiments, the peptide is 14 amino acids. In some embodiments, the peptide is 15 amino acids. In some embodiments, the peptide is 16 amino acids. In some embodiments, the peptide is 17 amino acids. In some embodiments, the peptide is 18 amino acids. In some embodiments, the peptide is 19 amino acids. In some embodiments, the peptide is 20 amino acids. In some embodiments, the peptide is 21 amino acids. In some embodiments, the peptide is 22 amino acids. In some embodiments, the peptide is 23 amino acids. In some embodiments, the peptide is 24 amino acids. In some embodiments, the peptide is 25 amino acids.

The BBB-R antigen to be used for production of, or screening for, peptide may be a soluble form of or a portion thereof (e.g. the extracellular domain) containing the desired epitope. The BBB-R target molecules may be isolated from natural sources or prepared by recombinant methods by procedures known in the art. The purified target molecule can be attached to a suitable matrix such as agarose beads, acrylamide beads, glass beads, cellulose, various acrylic copolymers, hydroxyalkyl methacrylate gels, polyacrylic and polymethacrylic copolymers, nylon, neutral and ionic carriers, and the like. Attachment of the target protein to the matrix may be accomplished by methods described in Methods in Enzymology, 44 1976, or by other means known in the art. Alternatively, or additionally, cells expressing BBB-R at their cell surface can be used to generate, or screen for, binding molecules. In some embodiments, binding molecules can be screened for membrane translocation activity can be

Provided in some aspects are methods of selecting or identifying peptides that bind to a BBB-R. In some embodiments, random peptides can be inserted into the backbone of a scaffold sequence, such as a cyclotide including any as described in Section I.B, and the scaffold library can be used to screen for binding to a BBB-R. Any known methods for generating libraries containing variant polynucleotides and/or polypeptides can be used with the provided methods and vectors to generate display libraries, e.g. phage display libraries, and to select binding proteins from the libraries. The libraries can be used in screening assays to select binding proteins from the library for binding to a BBB-R. To facilitate screening, libraries of variant binding molecules (e.g. cyclotides libraries inserted with random peptides) typically are screened using a display technique, such that there is a physical link between the individual molecules of the library (phenotype) and the genetic information encoding them (genotype). These methods include, but are not limited to, cell display, including bacterial display, yeast display, mammalian display, phage display (Smith, G. P. (1985) Science 228:1315-1317), mRNA display, ribosome display and DNA display.

In some embodiments, a library of cyclotides each inserted with a random peptide is screened to identify individual members of the library that exhibit a desired biological activity such as binding to a BBB-R. A method for screening for BBB-R binding, according to one embodiment, includes the steps of:
a) constructing a cyclotide sequence display library in which random peptides are inserted into the cyclotide scaffold (e.g. into a loop therein, e.g. loop 1);
b) expressing the sequence library in order to obtain expressed cyclotides, or polypeptides that comprise the cyclotides;
c) selecting the expressed sequence library against a suitable purified BBB-R;
d) recovering any cyclotide binding to the receptor;
e) determining the sequence of the recovered cyclotide or peptide contained therein.

For instance, the display library is a phage display library of a plurality of modified cyclotide scaffold (or other binding molecule scaffold) in which each is inserted with a random peptide. In some embodiments, each modified cyclotide scaffold of the library is fused to a phage coat protein and displayed, usually on average as a single copy of each related polypeptide, on the surface of a phagemid particle containing DNA encoding that polypeptide. These phagemid particles are then contacted with a BBB-R target and those particles having the highest affinity for the target are separated from those of lower affinity. The higher affinity binders are then amplified by infection of a bacterial host and the competitive binding step is repeated. This process is reiterated until polypeptides of the desired affinity are obtained.

In some embodiments, the provided methods include contacting any of the display libraries provided herein with a target molecule under conditions to allow binding of a display particle, e.g., a phagemid particle, to the target molecule. In some embodiments, the methods further include separating the display particles, e.g., the phagemid particles, that bind from those that do not, thereby selecting display particles, e.g., the phagemid particles, that include an antibody binding protein that binds to the target molecule. In some embodiments, the methods include sequencing the fusion gene in the selected particles to identify the antibody binding protein.

In some embodiments, the BBB-R target molecule is contacted with the library of display particles, e.g., phagemid particles, under conditions suitable for binding of at least a portion of the display particles with the target molecules. Normally, the conditions, including pH, ionic strength, temperature and the like will mimic physiological conditions. Exemplary “contacting” conditions may comprise incubation for 15 minutes to 4 hours, e.g. one hour, at 4°−37° C., e.g. at room temperature. However, these may be varied as appropriate depending on the nature of the interacting binding partners, etc. The mixture can be subjected to gentle rocking, mixing, or rotation. In addition, other appropriate reagents such as blocking agents to reduce nonspecific binding may be added. For example 14% BSA or other suitable blocking agent (e.g. milk) may be used. It will be appreciated however that the contacting conditions can be varied and adapted by a skilled person depending on the aim of the screening method. For example, if the incubation temperature is, for example, room temperature or 37° C., this may increase the possibility of identifying binders which are stable under these conditions, e.g., in the case of incubation at 37° C., are stable under conditions found in the human body. Such a property might be extremely advantageous if one or both of the binding partners was a candidate to be used in some sort of therapeutic application, e.g. an antibody. Again such adaptations to the conditions are within the ambit of the skilled person

Bound display particles (“binders”) having high affinity for the immobilized target molecule can be separated from those having a low affinity (and thus do not bind to the target) by washing. Binders can be dissociated from the immobilized target molecules by a variety of methods. These methods include competitive dissociation using the wild-type ligand, altering pH and/or ionic strength, and methods known in the art.

In another embodiments, peptides can be screened for their activity to confer blood brain barrier translocation activity. In such an embodiment, a library of cyclotides each inserted with a random peptide is screened to identify individual members of the library that exhibit a desired biological activity such as membrane translocation activity, and in particular transfer across the BBB when delivered to an animal. By “transfer across the blood-brain barrier” or “crossing the blood-brain barrier” or other variations thereof it is meant that the polypeptide is delivered to an animal and is capable of passing into the brain of the animal by traversing blood vessel walls in the brain. A method for screening for BBB transfer, according to one embodiment, includes the steps of:
a) constructing a cyclotide sequence display library in which random peptides are inserted into the cyclotide scaffold (e.g. into a loop therein, e.g. loop 1);
b) expressing the sequence library in order to obtain expressed cyclotides, or polypeptides that comprise the cyclotides;
c) administering the expressed cyclotides or polypeptides that comprise the cyclotides to an animal;
d) recovering any cyclotide from the brain of the mammal;
e) determining the sequence of the recovered cyclotide or peptide contained thereins.

The animal used in such a screen is typically a bird or mammal, and may be selected from humans, primates, cattle, sheep, rodents, cats, dogs, and rabbits. In the case of non-human animals the library of cyclotides may be suitably administered by oral gavage or inclusion within normal animal feed. Recovery of the cyclotides from the body of the animal may be via biopsy, or in the case of non-human animals via sacrifice of the animal and histological and pathological analysis of the tissues in the body. In this way it is also possible to identify members of the library of cyclotides that exhibit tissue specificity and/or the availability to cross various additional barriers within the body of the animal. By way of example, modified cyclotides that are found in a brain biopsy of a screened non-human animal would be considered as demonstrating the ability of being able to cross the BBB. In a specific embodiment of the invention the cyclotides of the invention are comprised within a phage display library and the determination of BBB transfer is made by analysing which cyclotides are capable of facilitating transport of an associated phage particle as a whole into the brain of an animal which has been fed or intravenously injected with at least a part of the phage display library.

Once one or more sets of cyclotides or peptides have been selected or isolated in accordance with the provided methods, these can be subjected to further analysis. In some embodiments, the further analysis involves the isolation of cyclotides by infection of bacteria as an amplification step, isolating the phage or phagemid DNA, and cloning the DNA sequence encoding the candidate cyclotides contained in said phage or phagemid DNA into a suitable expression vector. Such an infection step can also allow the amplification of the cyclotides. Alternatively, cyclotides can be amplified at this stage by other appropriate methods, for example by PCR of the nucleic acids encoding said cyclotides or the transformation of said nucleic acid into an appropriate host cell (in the context of a suitable expression vector).

Once the DNA encoding the cyclotides are cloned in a suitable expression vector, the DNA encoding the cyclotides can be sequenced or the protein can be expressed in a soluble form, e.g., including according to the methods provided herein, and subjected to appropriate binding studies to further characterize the candidates at the protein level. In some embodiments, the peptide contained in a selected cyclotide can be identified and can be assessed for binding to the BBB-R. Appropriate binding studies will depend on the nature of the binders, and include, but are not limited to ELISA, filter screening assays, FACS, or immunofluorescence assays, BiaCore affinity measurements or other methods to quantify binding constants, staining tissue slides or cells and other immunohistochemistry methods. One or more of these binding studies can be used to analyze the binders.

In some embodiments, identified peptides can be modified by histidine or alanine scanning to identify peptides with altered binding or affinity. For instance, a library of mutant peptides can be generated and binding of each variant for the BBB-R target molecule can be assessed. Any binding assay known to a skilled artisan can be used, including any as described above. In some embodiments, histidine scanning is carried out to mutate or vary amino acid residues in an identified peptide to histidine. In some embodiments, histidine scanning can be used to identify pH-sensitive antigen binding, for example to identify peptides that have reduced binding at acidic pH compared to neutral pH. Without wishing to be bound by theory, it is desirable that a receptor-binding peptide bind to a BBB-R at neutral pH (e.g. pH 7.0-7.4) but not bind or have reduced binding at pH 5.0 such as present in an endosome so that the binding molecule containing the peptide (e.g. modified cyclotide) is released into the blood-brain barrier and not recycled. In some embodiments, alanine scanning is carried out to mutate or vary amino acid residues in an identified peptide to alanine. In some embodiments, alanine scanning is useful to identify residues that can be mutated with retention of binding function.

Also provided herein are methods for identifying a peptide that binds to a BBB-R by amino acid sequence, including from a sequence library.

In some embodiments, the peptide is 6 to 50 amino acids in length and comprises the sequence set forth in any one of SEQ ID NOS: 26-34 or 49-54. In some embodiments, the peptide is 6 to 30 amino acids, 6 to 24 amino acids, 6 to 18 amino acids, 6 to 12 amino acids, 10 to 30 amino acids, 10 to 24 amino acids, 10 to 18 amino acids, 10 to 12 amino acids, 12 to 30 amino acids, 12 to 24 amino acids, 12 to 18 amino acids, 18 to 30 amino acids, 18 to 24 amino acids or 24 to 30 amino acids and comprises the sequence set forth in any one of SEQ ID NOS: 26-34 or 49-54. In some embodiments, the peptide is 12 to 18 amino acids in length, and comprises the sequence set forth in SEQ ID NO: 26. In some embodiments, the peptide is 12 to 18 amino acids in length, and comprises the sequence set forth in SEQ ID NO: 49. In some embodiments, the peptide is set forth in any one of SEQ ID NOS: 26-34 or 49-54. In some embodiments, the peptide is set forth in SEQ ID NO:26. In some embodiments, the peptide is set forth in SEQ ID NO: 49. In some embodiments, the peptide moiety binds the transferrin receptor (TrfR). In some embodiments, the TrfR is mouse. In some embodiments, the TrfR is human. In some embodiments, the peptide has blood-brain barrier translocation activity.

In some embodiments, the peptide is 6 to 50 amino acids in length and comprises a sequence that has at least 85%, at least 90%, at least 95% amino acid sequence identity to the sequence set forth in any one of SEQ ID NOS: 26-34 or 49-54. In some embodiments, the peptide is 6 to 50 amino acids in length and comprises a sequence that has 1, 2, 3 or 4 amino acid substitutions compared to the sequence set forth in any one of SEQ ID NOS: 26-34 or 49-54. In some embodiments, the peptide is 12 to 18 amino acids in length, and comprises a sequence that has 1, 2, 3 or 4 amino acid substitutions compared to the sequence set forth in any one of SEQ ID NOS: 26-34 or 49-54. In some embodiments, the amino acid substitution(s) is substitution of an amino acid residue to an alanine. In some embodiments, the amino acid substitution(s) is substitution of an amino acid residue to histidine. In some embodiments, the peptide moiety binds the transferrin receptor (TrfR). In some embodiments, the TrfR is mouse. In some embodiments, the TrfR is human. In some embodiments, the peptide has blood-brain barrier translocation activity.

In some embodiments, the peptide is 6 to 50 amino acids in length and comprises a sequence that has at least 85%, at least 90%, at least 95% amino acid sequence identity to the sequence set forth in any one of SEQ ID NO: 26. In some embodiments, the peptide is 6 to 50 amino acids in length and comprises a sequence that has 1, 2, 3 or 4 amino acid substitutions compared to the sequence set forth in any one of SEQ ID NOS: 26. In some embodiments, the amino acid substitution(s) is substitution of an amino acid residue to an alanine. In some embodiments, the amino acid substitution(s) is substitution of an amino acid residue to histidine. In some embodiments, the peptide is 6 to 50 amino acids in length and comprises the sequence set forth in any one of SEQ ID NOS: 55 or 117-128. In some embodiments, the peptide is 6 to 30 amino acids, 6 to 24 amino acids, 6 to 18 amino acids, 6 to 12 amino acids, 10 to 30 amino acids, 10 to 24 amino acids, 10 to 18 amino acids, 10 to 12 amino acids, 12 to 30 amino acids, 12 to 24 amino acids, 12 to 18 amino acids, 18 to 30 amino acids, 18 to 24 amino acids or 24 to 30 amino acids and comprises the sequence set forth in any one of SEQ ID NOS: 55 or 117-128. In some embodiments, the peptide is 12 to 18 amino acids in length, and comprises the sequence set forth in SEQ ID NO: 55. In some embodiments, the peptide is set forth in any one of SEQ ID NOS: 55 or 117-128. In some embodiments, the peptide is set forth in SEQ ID NO:55. In some embodiments, the peptide moiety binds the transferrin receptor (TrfR). In some embodiments, the TrfR is mouse. In some embodiments, the TrfR is human. In some embodiment, the peptide has blood-brain barrier translocation activity.

In some embodiments, the peptide is 6 to 50 amino acids in length and comprises the sequence set forth in any one of SEQ ID NOS: 10-23. In some embodiments, the peptide is 6 to 30 amino acids, 6 to 24 amino acids, 6 to 18 amino acids, 6 to 12 amino acids, 10 to 30 amino acids, 10 to 24 amino acids, 10 to 18 amino acids, 10 to 12 amino acids, 12 to 30 amino acids, 12 to 24 amino acids, 12 to 18 amino acids, 18 to 30 amino acids, 18 to 24 amino acids or 24 to 30 amino acids and comprises the sequence set forth in any one of SEQ ID NOS: 10-23. In some embodiments, the peptide is set forth in any one of SEQ ID NOS: 10-23. In some embodiments, the peptide moiety binds the leptin receptor (ObR). In some embodiments, the ObR is human. In some embodiment, the peptide has blood-brain barrier translocation activity.

In some embodiments, the peptide is 6 to 50 amino acids in length and comprises a sequence that has at least 85%, at least 90%, at least 95% amino acid sequence identity to the sequence set forth in any one of SEQ ID NOS: 10-23. In some embodiments, the peptide is 6 to 50 amino acids in length and comprises a sequence that has 1, 2, 3 or 4 amino acid substitutions compared to the sequence set forth in any one of SEQ ID NOS: 10-23. In some embodiments, the peptide is 12 to 18 amino acids in length, and comprises a sequence that has 1, 2, 3 or 4 amino acid substitutions compared to the sequence set forth in any one of SEQ ID NOS: 210-23. In some embodiments, the amino acid substitution(s) is substitution of an amino acid residue to an alanine. In some embodiments, the amino acid substitution(s) is substitution of an amino acid residue to histidine. In some embodiments, the peptide moiety binds the leptin receptor (ObR). In some embodiments, the ObR is human. In some embodiments, the peptide has blood-brain barrier translocation activity.

In some embodiments, the peptide is 6 to 50 amino acids in length and comprises the sequence set forth in any one of SEQ ID NOS: 24 or 25. In some embodiments, the peptide is 6 to 30 amino acids, 6 to 24 amino acids, 6 to 18 amino acids, 6 to 12 amino acids, 10 to 30 amino acids, 10 to 24 amino acids, 10 to 18 amino acids, 10 to 12 amino acids, 12 to 30 amino acids, 12 to 24 amino acids, 12 to 18 amino acids, 18 to 30 amino acids, 18 to 24 amino acids or 24 to 30 amino acids and comprises the sequence set forth in any one of SEQ ID NOS: 24 or 25. In some embodiments, the peptide is set forth in SEQ ID NO: 24. In some embodiments, the peptide is set forth in SEQ ID NO: 25. In some embodiments, the peptide moiety binds ErbB3. In some embodiments, the ErBB3 is human. In some embodiment, the peptide has blood-brain barrier translocation activity.

In some embodiments, the peptide is 6 to 50 amino acids in length and comprises a sequence that has at least 85%, at least 90%, at least 95% amino acid sequence identity to the sequence set forth in any one of SEQ ID NOS: 24 or 25. In some embodiments, the peptide is 6 to 50 amino acids in length and comprises a sequence that has 1, 2, 3 or 4 amino acid substitutions compared to the sequence set forth in any one of SEQ ID NOS: 24 or 25. In some embodiments, the peptide is 12 to 18 amino acids in length, and comprises a sequence that has 1, 2, 3 or 4 amino acid substitutions compared to the sequence set forth in any one of SEQ ID NOS: 24 or 25. In some embodiments, the amino acid substitution(s) is substitution of an amino acid residue to an alanine. In some embodiments, the amino acid substitution(s) is substitution of an amino acid residue to histidine. In some embodiments, the peptide moiety binds the ErbB3. In some embodiments, the ErbB3 is human. In some embodiments, the peptide has blood-brain barrier translocation activity.

In some embodiments, the peptide is 6 to 50 amino acids in length and comprises the sequence set forth in any one of SEQ ID NOS: 35 or 36. In some embodiments, the peptide is 6 to 30 amino acids, 6 to 24 amino acids, 6 to 18 amino acids, 6 to 12 amino acids, 10 to 30 amino acids, 10 to 24 amino acids, 10 to 18 amino acids, 10 to 12 amino acids, 12 to 30 amino acids, 12 to 24 amino acids, 12 to 18 amino acids, 18 to 30 amino acids, 18 to 24 amino acids or 24 to 30 amino acids and comprises the sequence set forth in any one of SEQ ID NOS: 35 or 36. In some embodiments, the peptide is 12 to 18 amino acids in length, and comprises the sequence set forth in SEQ ID NO:35. In some embodiments, the peptide is set forth in SEQ ID NO: 35. In some embodiments, the peptide is 12 to 18 amino acids in length, and comprises the sequence set forth in SEQ ID NO:36. In some embodiments, the peptide is set forth in any one of SEQ ID NO: 36. In some embodiments, the peptide moiety binds IGFR. In some embodiments, the IGFR is human. In some embodiment, the peptide has blood-brain barrier translocation activity.

In some embodiments, the peptide is 6 to 50 amino acids in length and comprises a sequence that has at least 85%, at least 90%, at least 95% amino acid sequence identity to the sequence set forth in any one of SEQ ID NOS: 35 or 36. In some embodiments, the peptide is 6 to 50 amino acids in length and comprises a sequence that has 1, 2, 3 or 4 amino acid substitutions compared to the sequence set forth in any one of SEQ ID NOS: 35 or 36. In some embodiments, the peptide is 12 to 18 amino acids in length, and comprises a sequence that has 1, 2, 3 or 4 amino acid substitutions compared to the sequence set forth in any one of SEQ ID NOS: 35 or 36. In some embodiments, the amino acid substitution(s) is substitution of an amino acid residue to an alanine. In some embodiments, the amino acid substitution(s) is substitution of an amino acid residue to histidine. In some embodiments, the peptide moiety binds the IGFR. In some embodiments, the IGFR is human. In some embodiments, the peptide has blood-brain barrier translocation activity.

In some embodiments, the peptide is 6 to 50 amino acids in length and comprises the sequence set forth in any one of SEQ ID NOS: 37 or 38. In some embodiments, the peptide is 6 to 30 amino acids, 6 to 24 amino acids, 6 to 18 amino acids, 6 to 12 amino acids, 10 to 30 amino acids, 10 to 24 amino acids, 10 to 18 amino acids, 10 to 12 amino acids, 12 to 30 amino acids, 12 to 24 amino acids, 12 to 18 amino acids, 18 to 30 amino acids, 18 to 24 amino acids or 24 to 30 amino acids and comprises the sequence set forth in any one of SEQ ID NOS: 37 or 38. In some embodiments, the peptide is 12 to 18 amino acids in length, and comprises the sequence set forth in SEQ ID NO:37. In some embodiments, the peptide is set forth in SEQ ID NO: 37. In some embodiments, the peptide is 12 to 18 amino acids in length, and comprises the sequence set forth in SEQ ID NO:38. In some embodiments, the peptide is set forth in SEQ ID NO: 38. In some embodiments, the peptide moiety binds RAGE. In some embodiments, the RAGE is human. In some embodiment, the peptide has blood-brain barrier translocation activity.

In some embodiments, the peptide is 6 to 50 amino acids in length and comprises a sequence that has at least 85%, at least 90%, at least 95% amino acid sequence identity to the sequence set forth in any one of SEQ ID NOS: 37 or 38. In some embodiments, the peptide is 6 to 50 amino acids in length and comprises a sequence that has 1, 2, 3 or 4 amino acid substitutions compared to the sequence set forth in any one of SEQ ID NOS: 37 or 38. In some embodiments, the peptide is 12 to 18 amino acids in length, and comprises a sequence that has 1, 2, 3 or 4 amino acid substitutions compared to the sequence set forth in any one of SEQ ID NOS: 37 or 38. In some embodiments, the amino acid substitution(s) is substitution of an amino acid residue to an alanine. In some embodiments, the amino acid substitution(s) is substitution of an amino acid residue to histidine. In some embodiments, the peptide moiety binds the RAGE. In some embodiments, the RAGE is human. In some embodiment, the peptide has blood-brain barrier translocation activity.

In some embodiments, the peptide is 6 to 50 amino acids in length and comprises the sequence set forth in any one of SEQ ID NOS: 39-48. In some embodiments, the peptide is 6 to 30 amino acids, 6 to 24 amino acids, 6 to 18 amino acids, 6 to 12 amino acids, 10 to 30 amino acids, 10 to 24 amino acids, 10 to 18 amino acids, 10 to 12 amino acids, 12 to 30 amino acids, 12 to 24 amino acids, 12 to 18 amino acids, 18 to 30 amino acids, 18 to 24 amino acids or 24 to 30 amino acids and comprises the sequence set forth in any one of SEQ ID NOS: 39-48. In some embodiments, the peptide is set forth in any one of SEQ ID NOs: 39-48. In some embodiments, the peptide is 12 to 18 amino acids in length, and comprises the sequence set forth in SEQ ID NO:39. In some embodiments, the peptide is set forth in SEQ ID NO: 39. In some embodiments, the peptide is 12 to 18 amino acids in length, and comprises the sequence set forth in SEQ ID NO:41. In some embodiments, the peptide is set forth in SEQ ID NO: 41. In some embodiments, the peptide is 12 to 18 amino acids in length, and comprises the sequence set forth in SEQ ID NO:43. In some embodiments, the peptide is set forth in SEQ ID NO: 43. In some embodiments, the peptide moiety binds LRP-1. In some embodiments, the LRP-1 is human. In some embodiment, the peptide has blood-brain barrier translocation activity.

In some embodiments, the peptide is 6 to 50 amino acids in length and comprises a sequence that has at least 85%, at least 90%, at least 95% amino acid sequence identity to the sequence set forth in any one of SEQ ID NOS: 39-48. In some embodiments, the peptide is 6 to 50 amino acids in length and comprises a sequence that has 1, 2, 3 or 4 amino acid substitutions compared to the sequence set forth in any one of SEQ ID NOS: 39-48. In some embodiments, the peptide is 12 to 18 amino acids in length, and comprises a sequence that has 1, 2, 3 or 4 amino acid substitutions compared to the sequence set forth in any one of SEQ ID NO: 39, 41 or 43. In some embodiments, the amino acid substitution(s) is substitution of an amino acid residue to an alanine. In some embodiments, the amino acid substitution(s) is substitution of an amino acid residue to histidine. In some embodiments, the peptide moiety binds the LRP-1. In some embodiments, the LRP-1 is human. In some embodiment, the peptide has blood-brain barrier translocation activity.

In some of any of the preceding embodiments, the peptide has blood-brain barrier translocation activity. In some of any of the preceding embodiments, the peptide binds to a receptor involved in blood-brain barrier transcytosis.

B. Binding Molecules (e.g. Cyclotide) Scaffolds and Modified Cyclotides

Also provided herein are binding molecules that comprise a peptide that binds to a BBB-R, including any set forth in Section I.A. above. In some embodiments, the binding molecule provides a stable scaffold (also interchangeably used with the term “backbone”) into which the peptide can be inserted. In some embodiments, the binding molecule is a cysteine-knot protein. In some embodiments, the binding molecule is a cyclotide.

The technique of using a protein “scaffold” and the engineering of loops or regions within the scaffold to alter activity is most notable with regard to the field of antibodies and antibody fragments, which have a natural repertoire of variable regions or loops. The variable loops of antibodies have been extensively engineered to produce peptides having improved binding (e.g. affinity and/or specificity) to known ligands, and also to expand the binding substrates for particular antibody frameworks (see for example, Knappik et al., (2000) J. Mol. Biol., 296, 57-86; and EP 1025218). The engineering of non-antibody frameworks has been reviewed, for example, by Hosse et al., (2006), Protein Sci., 15, 14-27. Such non-antibody or alternative scaffold proteins have considerable advantages over traditional antibodies due to their small size, high stability, and ability to be expressed in prokaryotic hosts. Suitable scaffolds may include modified whey acidic protein (WAP) domain containing polypeptides, such as those described in International patent application published as WO-A-2012073045. It will be appreciated by the skilled person that suitable scaffolds are not limited solely to the WAP domain of human elafin (trappin-2) but may include other human trappins (e.g. SLPI) or non-human trappins (e.g. from porcine, bovine or simian sources). In addition, the provided embodiments extends to cyclotides comprised within other non-trappin members of the WAP domain family. Further, provided embodiments also relate to employing plant cyclotides as scaffolds.

In some embodiments, the molecules provided herein that employ cysteine-knot proteins, such as cyclotides, as scaffolds for insertion of the peptides may be used as carriers for delivery of biological agents, such as protein therapeutics, across the blood brain barrier. In some embodiments, the cyclotide is a linear or cyclic amino acid sequence containing a structure referred to herein as a “cysteine knot”. A cysteine knot occurs when a disulfide bond passes through a closed cyclic loop formed by two other disulfide bonds and the amino acids in the backbone. However, reference herein to a “cysteine knot” includes reference to structural equivalents thereof which provide similar constraints to the three-dimensional structure of the cyclic backbone. For example, appropriate turns and loops in the “cysteine-knot” backbone may also be achieved by engineering suitable covalent bonds or other forms of molecular associations. All such modifications to the cyclic backbone which result in retention of the three-dimensional knotted topology conferred by the cysteine knot are encompassed by the provided embodiments. Furthermore, although a cysteine knot is characterized by a knot formed by three disulfide bonds, the provided embodiments extends to molecules comprising only two disulfide bonds. In such a case, the molecular framework may need to be further stabilized using other means or the molecular framework may retain suitable activity despite a change in three-dimensional structure caused by the absence of a third disulfide bond. In yet a further modification, the cysteine knot backbone may comprise more than three disulfide bonds such as occurring in a double or multiple cysteine knot arrangement or in a single cysteine knot arrangement supplement by one or two additional disulfide bonds.

Cysteine-knot microproteins (such as cyclotides) include a naturally occurring family of cysteine-knot microproteins or cyclotides found in various plant species. Cysteine-knot microproteins (cyclotides) are small peptides, typically consisting of about 30-40 amino acids, which can be found naturally as cyclic or linear forms, where the cyclic form has no free N- or C-terminal amino or carboxyl end. They have a defined structure based on three intra-molecular disulfide bonds and a small triple stranded β-sheet (Craik et al., 2001; Toxicon 39, 43-60). The cyclic proteins exhibit conserved cysteine residues defining a structure referred to herein as a “cysteine knot”. This family includes both naturally occurring cyclic molecules and their linear derivatives as well as linear molecules which have undergone cyclization. These molecules are useful as molecular framework structures having enhanced stability over less structured peptides. (Colgrave and Craik, 2004; Biochemistry 43, 5965-5975). However, these molecules are not themselves capable of crossing the blood-brain barrier to any great extent, and cannot be used as neuro-therapeutic agents themselves or act as carriers of therapeutic peptide or protein agents.

The main cyclotide features are a remarkable stability due to the cysteine knot, a small size making them readily accessible to chemical synthesis, and an excellent tolerance to sequence variations. The provided embodiments are based on the recognition herein that cyclotides therefore appear as appealing leads or scaffolds for peptide drug design. The cyclotide scaffold is found in almost 30 different protein families among which conotoxins, spider toxins, squash inhibitors, agouti-related proteins and plant cyclotides are the most populated families. Cyclotides from plants in the Rubiaceae and Violaceae families are for the most part found to be head-to-tail cyclic peptides (Craik et al. 2010. Cell. Mol. Life Sci. 67:9-16). However, within the squash inhibitor family of cyclotides both cyclic and linear cyclotides have been identified from Momordica cochinchinensis: the cyclic trypsin inhibitors (MCoTI)-I and —II and their linear counterpart MCoTI-III (Hernandez et al. 2000. Biochemistry, 39, 5722-5730). It is now clear that both cyclic and linear variants can exist in different cyclotide families, but the impact of the cyclization is poorly understood. Cyclic peptides were expected to display improved stability, better resistance to proteases, and reduced flexibility when compared to their linear counterparts, hopefully resulting in enhanced biological activities. However, linear cyclotides have the advantage of being able to be more easily linked to other peptides or proteins.

For instance, cyclotides are commonly found in plants. In aspects of provided embodiments, cyclotides of the invention are derived from linear or cyclic form of cyclotides of the Momordicae, Rubiaceae and Violaceae, plant species. In a preferred aspect, cyclotides of the invention are derived from linear or cyclic form of cyclotides of the Momordicae species including the squash serine protease inhibitor family (Otlewski & Korowarsch Acta Biochim Pol. 1996; 43(3):431-44) and in a more preferred aspect from Momordica cochinchinensis trypsin inhibitors. In some embodiments, the Momordica cochinschinenis trypsin inhibitor include inhibitors MCoTI-I (SEQ ID NO: 1) and -II (SEQ ID NO: 2) (naturally cyclic) and MCoTI-III (naturally linear) (SEQ ID NO: 3) below.

Mcoti-I
(SEQ ID NO: 1)
GGVCPKILQRCRRDSDSPGACICRGNGYCGSGSD
Mcoti-II
(SEQ ID NO: 2)
GGVCPKILKKCRRDSDSPGACICRGNGYCGSGSD
Mcoti-III
(SEQ ID NO: 3)
ERACPRILKKCRRDSDSPGACICRGNGYCG

Provided herein are modified cyclotides containing a peptide of 2 to 50 amino acids that binds to a BBB-R. In some embodiments, the peptide is any as described in Section I.A. In some embodiments, the peptide is inserted into a loop of the unmodified or wild-type cyclotide. In some embodiments, a modified cyclotide sequence provided herein may be defined as having a cysteine knot backbone moiety and a peptide that is a blood-brain barrier translocation moiety, said modified cyclotide comprising: i) a peptide that binds a BBB-R, wherein said peptide is about 2 to 50 amino acid residues length; and ii) a cysteine knot backbone grafted to said peptide of clause i), wherein said cysteine knot backbone comprises the structure (I):

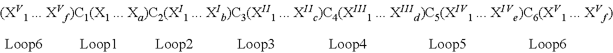

wherein C₁ to C₆ are cysteine residues; wherein each of C₁ and C₄, C₂ and C₅, and C₃ and C₆ are connected by a disulfide bond to form a cysteine knot; wherein each X represents an amino acid residue in a loop, wherein said amino acid residues are the same or different; wherein d is about 1-2; wherein one or more of loops 1, 2, 3, 5 or 6 have an amino acid sequence comprising the sequence of said peptide of clause i), wherein any loop comprising said sequence of said peptide of clause i) comprises 2 to about 50 amino acids, and wherein for any of loops 1, 2, 3, 5, or 6 that do not contain said sequence of said peptide of clause i), a, b, c, e, and f, are the same or different, and are each any number from 3-10, and b, c, e, and f are each any number from 1 to 20. In some embodiments, the peptide is any of the peptides described in Section I.A. In some embodiments, the peptide is any of the peptides set forth in Table E1 or Table E2.

In some embodiments, the unmodified or wildtype cyclotide can be a cyclotide set forth in any one of SEQ ID NOS:1-3 to which one or more loops thereof is inserted or substituted by one or more amino acid sequences, e.g. a peptide of 2 to 50 amino acids, such as any described in Section I.A. In some embodiments, the unmodified or wildtype cyclotide can be a cyclotide set forth in any one of SEQ ID NO:1 to which one or more loops thereof is inserted or substituted by one or more amino acid sequences, e.g. a peptide of 2 to 50 amino acids, such as any described in Section I.A. In some embodiments, the unmodified or wildtype cyclotide can be a cyclotide set forth in SEQ ID NO:2 to which one or more loops thereof is inserted or substituted by one or more amino acid sequences, e.g. a peptide of 2 to 50 amino acids, such as any described in Section I.A. In some embodiments, the unmodified or wildtype cyclotide can be a cyclotide set forth in any one of SEQ ID NO:3 to which one or more loops thereof is inserted or substituted by one or more amino acid sequences, e.g. a peptide of 2 to 50 amino acids, such as any described in Section I.A. In some embodiments, the provided modified cyclotides of the invention exhibit improved or enhanced binding to a receptor involved in transcytosis across the blood-brain barrier, such as ta BBB-R, e.g. the human transferrin receptor. In some embodiments, the provided modified cyclotides possess enhanced blood-brain barrier translocation characteristics. The improvement or enhancement is as compared to the unmodified cyclotide from which the modified cyclotide is derived, such as a wild-type or natural cyclotide, including a cyclotide set forth in SEQ ID NO: 1, SEQ ID NO: 2 or SEQ ID NO: 3.

The provided cyclotides contain a sequence of amino acids or analogues thereof forming a cysteine-knot backbone wherein said cysteine-knot backbone comprises sufficient disulfide bonds or chemical equivalents thereof, to confer a knotted topology on the three-dimensional structure of said cysteine-knot backbone and wherein at least one exposed amino acid residue such as on one or more beta turns and/or within one or more loops, is inserted or substituted relative to the naturally occurring cyclotide amino acid sequence. In some embodiments, the inserted or substituted loop is loop 1 of a native or naturally occurring cyclotide sequence. In some embodiments, the inserted or substituted loop is loop 2 of a native or naturally occurring cyclotide sequence. In some embodiments, the inserted or substituted loop is loop 3 of a native or naturally occurring cyclotide sequence. In some embodiments, the inserted or substituted loop is loop 5 of a native or naturally occurring cyclotide sequence. In some embodiments, the inserted or substituted loop is loop 6 of a native or naturally occurring cyclotide sequence. In some embodiments, the provided cyclotides exhibit enhanced translocation behaviour compared with the unmodified or native parental cyclotide.

In particular embodiments, the provided modified cyclotides are derived from loop replacement libraries based on Mcoti-I (SEQ ID NO: 1). In particular embodiments, provided herein is a modified cyclotide comprising a blood-brain barrier translocation peptide moiety that is inserted into or that substitutes or replaces one or more amino acids of a loop of the cyclotide Mcoti-I (SEQ ID NO:1). In some embodiments, the peptide has an amino acid sequence that is about 2 to 50 amino acid residues, such as any described in Section I.A. In some embodiments, the loop into which the peptide is inserted or subtituted is loop 1. In some embodiments, the modified cyclotide has the sequence of SEQ ID NO:1 in which residues in loop 1 between the first two cysteine are replaced by the binding peptide. In some embodiments, the peptide is inserted between cysteine 4 (Cys4) and cysteine 11 (Cys11) of SEQ ID NO:1. In some embodiments, the loop into which the peptide is inserted or substituted is loop 5. In some embodiments, the modified cyclotide has the sequence of SEQ ID NO:1 in which residues in loop 5 between the last two cysteine are replaced by the binding peptide. In some embodiments, the peptide is inserted between cysteine 23 (Cys23) and cysteine 29 (Cys29) of SEQ ID NO:1. In some embodiments, the loop into which the peptide is inserted or substituted is loop 6, such as formed subject to cyclization. In some embodiments, the modified cyclotide has the sequence of SEQ ID NO:1 in which residues in loop 6 between the first and last cysteine (after cyclization) are replaced by the binding peptide. In some embodiments, the peptide is inserted between cysteine 29 (Cys29) and cysteine 4 (Cys4) of SEQ ID NO:1.

In particular embodiments, the provided modified cyclotides are derived from loop replacement libraries based on Mcoti-II (SEQ ID NO: 2). In particular embodiments, provided herein is a modified cyclotide comprising a blood-brain barrier translocation peptide moiety that is inserted into or that substitutes or replaces one or more amino acids of a loop of the cyclotide Mcoti-II (SEQ ID NO:2). In some embodiments, the peptide has an amino acid sequence that is about 2 to 50 amino acid residues, such as any described in Section I.A. In some embodiments, the loop into which the peptide is inserted or substituted is loop 1. In some embodiments, the modified cyclotide has the sequence of SEQ ID NO:2 in which residues in loop 1 between the first two cysteine are replaced by the binding peptide. In some embodiments, the peptide is inserted between cysteine 4 (Cys4) and cysteine 11 (Cys11) of SEQ ID NO:2. In some embodiments, the loop into which the peptide is inserted or substituted is loop 5. In some embodiments, the modified cyclotide has the sequence of SEQ ID NO:2 in which residues in loop 5 between the last two cysteine are replaced by the binding peptide. In some embodiments, the peptide is inserted between cysteine 23 (Cys23) and cysteine 29 (Cys29) of SEQ ID NO:2. In some embodiments, the loop into which the peptide is inserted or substituted is loop 6, such as formed subject to cyclization. In some embodiments, the modified cyclotide has the sequence of SEQ ID NO:2 in which residues in loop 6 between the first and last cysteine (after cyclization) are replaced by the binding peptide. In some embodiments, the peptide is inserted between cysteine 29 (Cys29) and cysteine 4 (Cys4) of SEQ ID NO:2. In particular embodiments, the provided modified cyclotides are derived from loop replacement libraries based on Mcoti-III (SEQ ID NO: 3). In particular embodiments, provided herein is a modified cyclotide comprising a blood-brain barrier translocation peptide moiety that is inserted into or that substitutes or replaces one or more amino acids of a loop of the cyclotide Mcoti-III (SEQ ID NO:3). In some embodiments, the peptide has an amino acid sequence that is about 2 to 50 amino acid residues, such as any described in Section I.A. In some embodiments, the loop into which the peptide is inserted or substituted is loop 1. In some embodiments, the modified cyclotide has the sequence of SEQ ID NO:3 in which residues in loop 1 between the first two cysteine are replaced by the binding peptide. In some embodiments, the peptide is inserted between cysteine 4 (Cys4) and cysteine 11 (Cys11) of SEQ ID NO:3. In some embodiments, the loop into which the peptide is inserted or substituted is loop 5. In some embodiments, the modified cyclotide has the sequence of SEQ ID NO:3 in which residues in loop 5 between the last two cysteine are replaced by the binding peptide. In some embodiments, the peptide is inserted between cysteine 23 (Cys23) and cysteine 29 (Cys29) of SEQ ID NO:3. In some embodiments, the loop into which the peptide is inserted or substituted is loop 6, such as formed subject to cyclization. In some embodiments, the modified cyclotide has the sequence of SEQ ID NO:3 in which residues in loop 6 between the first and last cysteine (after cyclization) are replaced by the binding peptide. In some embodiments, the peptide is inserted between cysteine 29 (Cys29) and cysteine 4 (Cys4) of SEQ ID NO:3.

In some embodiments, the peptide that is inserted or replaced into an unmodified cyclotide, e.g. the cyclotide Mcoti-I (SEQ ID NO: 1), is 2 to 50 amino acid residues. In some embodiments, the peptide that is inserted or replaced into an unmodified cyclotide, e.g. the cyclotide Mcoti-II (SEQ ID NO:2), is 2 to 50 amino acid residues. In some embodiments, the peptide that is inserted or replaced into an unmodified cyclotide, e.g. the cyclotide Mcoti-III (SEQ ID NO:3), is 2 to 50 amino acid residues. In some embodiments, the peptide is 2 to 40 amino acids, 2 to 30 amino acids, 2 to 25 amino acids, 2 to 20 amino acids, 2 to 15 amino acids, 2 to 10 amino acids, 2 to 5 amino acids, 5 to 50 amino acids, 5 to 40 amino acids, 5 to 30 amino acids, 5 to 25 amino acids, 5 to 20 amino acids, 5 to 15 amino acids, 5 to 10 amino acids, 10 to 50 amino acids, 10 to 40 amino acids, 10 to 30 amino acids, 10 to 25 amino acids, 10 to 15 amino acids, 15 to 50 amino acids, 15 to 40 amino acids, 15 to 30 amino acids, 15 to 25 amino acids, 15 to 20 amino acids, 20 to 50 amino acids, 20 to 40 amino acids, 20 to 30 amino acids, 20 to 25 amino acids, 25 to 50 amino acids, 25 to 40 amino acids, 25 to 30 amino acids, 30 to 50 amino acids, 30 to 40 amino acids, or 40 to 50 amino acids. In some embodiments, the peptide is 2 to 30 amino acids, such as 2 to 24 amino acids, 2 to 18 amino acids, 2 to 12 amino acids, 2 to 6 amino acids, 6 to 30 amino acids, 6 to 24 amino acids, 6 to 18 amino acids, 6 to 12 amino acids, 12 to 30 amino acids, 12 to 24 amino acids, 12 to 18 amino acids, 18 to 30 amino acids, 18 to 24 amino acids or 24 to 30 amino acids. In some embodiments, the peptide is 14 to 20 amino acids. In some embodiments, the peptide is 10 amino acids. In some embodiments, the peptide is 11 amino acids. In some embodiments, the peptide is 12 amino acids. In some embodiments, the peptide is 13 amino acids. In some embodiments, the peptide is 14 amino acids. In some embodiments, the peptide is 15 amino acids. In some embodiments, the peptide is 16 amino acids. In some embodiments, the peptide is 17 amino acids. In some embodiments, the peptide is 18 amino acids. In some embodiments, the peptide is 19 amino acids. In some embodiments, the peptide is 20 amino acids.

In some embodiments, a provided modified cyclotide has an amino acid sequence that exhibits at least 85% sequence identity to any of SEQ ID NOS: 56-101 or 105-116, wherein the modified cyclotide has blood-brain barrier translocation activity and/or binds to a receptor involved in blood-brain barrier transcytosis. In some embodiments, a provided modified cyclotide has an amino acid sequence that exhibits at least 90% sequence identity to any of SEQ ID NOS: 56-101 or 105-116, wherein the modified cyclotide has blood-brain barrier translocation activity and/or binds to a receptor involved in blood-brain barrier transcytosis. In some embodiments, a provided modified cyclotide has an amino acid sequence that exhibits at least 95% sequence identity to any of SEQ ID NOS: 56-101 or 105-116, wherein the modified cyclotide has blood-brain barrier translocation activity and/or binds to a receptor involved in blood-brain barrier transcytosis. In some embodiments, the sequence identity it at or about 86%, 87%, 88%, 89%, 90%, 91%, 92%, 93%, 94%, 95%, 96%, 97%, 98% or 99% to any of SEQ ID NOS: 56-101 or 105-116. In some of any of the above embodiments, the modified cyclotide binds to a receptor involved in blood-brain barrier transcytosis, such as a BBB-R. In some embodiments, the modified cyclotide has blood-brain barrier translocation activity, such as measured following subcutaneous administration to a subject. In some embodiments, the modified cyclotide binds to a receptor involved in blood-brain barrier transcytosis, i.e. BBB-R, and has blood-brain barrier translocation activity when administered subcutaneously to a subject.

In some of any of the preceding embodiments, the modified cyclotide binds to a BBB-R that is expressed on brain endothelial cells. In some embodiments, the BBB-R is selected from the transferrin receptor (TrfR); the lactoferrin receptor (LtfR); the leptin receptor (LEP-R, or also known as OB-R); the receptor tyrosine-protein kinase (ErbB3), a insulin receptor, such as Insulin Receptor A (IRA), IGF-1 Receptor (IGF-1R), IGF-II Receptor (IGF-IIR), RXFP1, RXFP2, RXFP3 and RXFP4; low-density lipoprotein receptor-related protein 1 (LRP-1), the receptor for advanced glycation end products (RAGE); heparin binding EGF like growth factor receptor (HB EGFR); the intercellular adhesion molecule 1 (ICAM1), intercellular adhesion molecule 2 (ICAM2) or neural cell adhesion molecule (NCAM); or any other receptor that has receptor-mediated transcytosis activity in brain endothelial cells.

In some embodiments, the modified cyclotides are derived from loop replacement libraries based on Mcoti-II (SEQ ID NO: 2) and bind to the transferrin receptor (e.g. human transferrin receptor). In some embodiments, the loop replacement is replacement of at least one loop, such as loop 1, with a peptide that comprises the sequence set forth in any one of SEQ ID NOS: 26-34 and 49-54. In some embodiments, the peptide is the peptide set forth in any one of SEQ ID NOS: 26-34 and 49-54. In some embodiments, the peptide is the peptide set forth in SEQ ID NO:26. In some embodiments, the peptide is the peptide set forth in SEQ ID NO: 49. In some embodiments, the modified cyclotide comprises the sequence set forth in any one of SEQ ID NOS: 72-80 and 95-100. In some embodiments, the modified cyclotide is set forth in any one of SEQ ID NOS: 72-80 and 95-100. In some embodiments, the modified cyclotide is set forth in SEQ ID NO:72. In some embodiments, the modified cyclotide is set forth in SEQ ID NO: 95. In some embodiments, the peptide contained in the modified cyclotide has 1, 2, 3 or 4 amino acid substitution(s) compared to the peptide set forth in any one of SEQ ID NOS: 26-34 and 49-54. In some embodiments, the amino acid substitution(s) is to a histidine. In some embodiments, the amino acid substitution(s) is to an alanine. In some embodiments, the modified cyclotide comprises the peptide sequence set forth in any one of SEQ ID NOS: 55 and 117-128. In some embodiments, the peptide sequence is set forth in any one of SEQ ID NOS: 55 and 117-128. In some embodiments, the modified cyclotide comprises the sequence set forth in any one of SEQ ID NOS: 101 and 105-116. In some embodiments, the modified cyclotide is set forth in any one of SEQ ID NOS: 101 and 105-116. In some embodiments, the peptide sequence is set forth in SEQ ID NO:55 and the modified cyclotide is set forth in SEQ ID NO:101.

In some embodiments, the modified cyclotide is a modified cyclotide that binds to the transferrin but has altered binding to transferrin receptor compared to the cyclotide set forth in any one of SEQ ID NOS: 72-80 and 95-100. In some embodiments, the modified cyclotide has altered binding to transferrin receptor compared to the cyclotide set forth in SEQ ID NOS: 72. In some embodiments, the modified cyclotide is a modified cyclotide that has reduced binding affinity to the transferrin receptor compared to the modified cyclotide set forth in SEQ ID NO:72, such as reduced by 1.5-fold, 2.0-fold, 3.0-fold, 4.0-fold, 5.0-fold, 10.0-fold or more. In some embodiments, the modified cyclotide has 1, 2, 3, 4 or 5 amino acid differences compared to the cyclotide set forth in SEQ ID NO:72. In some embodiments, the modified cyclotide has one or more amino acid substitutions compared to the cyclotide set forth in SEQ ID NO:72, wherein the one or more amino acid substitutions are selected from the group consisting of W9H, L11H, S13H, W14H and G15H. In some embodiments, the peptide is a peptide set forth in any one of SEQ ID NOS: 55, 122, 124, 126 or 127. In particular embodiments, the peptide is set forth in SEQ ID NO: 55. In some embodiments, the modified cyclotide is modified by loop replacement (e.g. loop 1) of Mcoti-II (SEQ ID NO: 2) with the peptide set forth in SEQ ID NO:122, for example the modified cyclotide comprises the sequence set froth in SEQ ID NO:110. In some embodiments, the modified cyclotide is modified by loop replacement (e.g. loop 1) of Mcoti-II (SEQ ID NO: 2) with the peptide set forth in SEQ ID NO:124, for example the modified cyclotide comprises the sequence set forth in SEQ ID NO:112. In some embodiments, the modified cyclotide is modified by loop replacement (e.g. loop 1) of Mcoti-II (SEQ ID NO: 2) with the peptide set forth in SEQ ID NO:126, for example the modified cyclotide comprises the sequence set forth in SEQ ID NO:114. In some embodiments, the modified cyclotide is modified by loop replacement (e.g. loop 1) of Mcoti-II (SEQ ID NO: 2) with the peptide set forth in SEQ ID NO:127, for example the modified cyclotide comprises the sequence set forth in SEQ ID NO: 115. In some embodiments, the modified cyclotide is modified by loop replacement (e.g. loop 1) of Mcoti-II (SEQ ID NO: 2) with the peptide set forth in SEQ ID NO:55, for example the modified cyclotide comprises the sequence set forth in SEQ ID NO:101. In some embodiments, the binding affinity to the transferrin receptor is pH-dependent, such that the modified cyclotide exhibits greater binding affinity to the transferrin receptor at neutral pH (e.g. 7.0-7.4) than acidic pH of the endosome (e.g. pH of 4.5 to 6.5, such as at or about pH 5.0). In some embodiments, such modified cyclotides exhibit enhanced blood-brain barrier translocation characteristics. Without wishing to be bound by theory, in some aspects a modified cyclotide with reduced binding to the transferrin receptor is released into the blood-brain barrier and not recycled back.

In some embodiments, the modified cyclotides are derived from loop replacement libraries based on Mcoti-II (SEQ ID NO: 2) and bind to the leptin receptor (e.g. human leptin receptor, also called Hu ObR). In some embodiments, the loop replacement is replacement of at least one loop, such as loop 1, with a peptide that comprises the sequence set forth in any one of SEQ ID NOS: 10-23. In some embodiments, the peptide is the peptide set forth in any one of SEQ ID NOS: 10-23. In some embodiments, the modified cyclotide comprises the sequence set forth in any one of SEQ ID NOS: 56-69. In some embodiments, the modified cyclotide is set forth in any one of SEQ ID NOS: 56-69.

In some embodiments, the modified cyclotide is a modified cyclotide that binds to the leptin receptor but has altered binding to the leptin receptor compared to the cyclotide set forth in any one of SEQ ID NOS: 56-69. In some embodiments, the binding affinity to the leptin receptor is reduced, such as reduced by 1.5-fold, 2.0-fold, 3.0-fold, 4.0-fold, 5.0-fole, 10.0-fold or more. In some embodiments, the binding affinity to the leptin receptor is pH-dependent, such that the modified cyclotide exhibits greater binding affinity to the leptin receptor at neutral pH (e.g. 7.0-7.4) than acidic pH of the endosome (e.g. pH of 4.5 to 6.5, such as at or about pH 5.0). In some embodiments, the modified cyclotide has a loop with a peptide that has 1, 2, 3 or 4 amino acid substitution(s) compared to the peptide set forth in any one of SEQ ID NOS: 10-23. In some embodiments, the modified cyclotide is modified by loop replacement (e.g. loop 1) of Mcoti-II (SEQ ID NO: 2) with a peptide that has 1, 2, 3 or 4 amino acid substitution(s) compared to the peptide set forth in any one of SEQ ID NOS: 10-23. In some embodiments, the amino acid substitution(s) is to a histidine. In some embodiments, the amino acid substitution(s) is to an alanine. In some embodiments, such modified cyclotides exhibit enhanced blood-brain barrier translocation characteristics.

In some embodiments, the modified cyclotides are derived from loop replacement libraries based on Mcoti-II (SEQ ID NO: 2) and bind to ErbB3 (e.g. human ErbB3). In some embodiments, the loop replacement is replacement of at least one loop, such as loop 1, with a peptide that comprises the sequence set forth in SEQ ID NO: 24 or 25. In some embodiments, the peptide is the peptide set forth in SEQ ID NO: 24 or 25. In some embodiments, the modified cyclotide comprises the sequence set forth in SEQ ID NO: 70 or 71. In some embodiments, the modified cyclotide is set forth in SEQ ID NOS: 70 or 71.

In some embodiments, the modified cyclotide is a modified cyclotide that binds to ErbB3 but has altered binding to ErB3 compared to the cyclotide set forth in SEQ ID NOS: 70 or 71. In some embodiments, the binding affinity to the ErbB3 is reduced, such as reduced by 1.5-fold, 2.0-fold, 3.0-fold, 4.0-fold, 5.0-fole, 10.0-fold or more. In some embodiments, the binding affinity to ErbB3 is pH-dependent, such that the modified cyclotide exhibits greater binding affinity to ErB3 at neutral pH (e.g. 7.0-7.4) than acidic pH of the endosome (e.g. pH of 4.5 to 6.5, such as at or about pH 5.0). In some embodiments, the modified cyclotide has a loop with a peptide that has 1, 2, 3 or 4 amino acid substitution(s) compared to the peptide set forth in SEQ ID NO: 24 or 25. In some embodiments, the modified cyclotide is modified by loop replacement (e.g. loop 1) of Mcoti-II (SEQ ID NO: 2) with a peptide that has 1, 2, 3 or 4 amino acid substitution(s) compared to the peptide set forth in any one of SEQ ID NOS: 24 or 25. In some embodiments, the amino acid substitution(s) is to a histidine. In some embodiments, the amino acid substitution(s) is to an alanine. In some embodiments, such modified cyclotides exhibit enhanced blood-brain barrier translocation characteristics.

In some embodiments, the modified cyclotides are derived from loop replacement libraries based on Mcoti-II (SEQ ID NO: 2) and bind to the IGFR (e.g. human IGFR). In some embodiments, the loop replacement is replacement of at least one loop, such as loop 1, with a peptide that comprises the sequence set forth in SEQ ID NO: 35 or 36. In some embodiments, the peptide is the peptide set forth in SEQ ID NOS: 35 or 36. In some embodiments, the peptide is the peptide set forth in SEQ ID NO:36. In some embodiments, the modified cyclotide comprises the sequence set forth in SEQ ID NO: 81 or 82. In some embodiments, the modified cyclotide is set forth in SEQ ID NO: 81 or 82. In some embodiments, the modified cyclotide is set forth in SEQ ID NO: 81. In some embodiments, the modified cyclotide is set forth in SEQ ID NO:82.

In some embodiments, the modified cyclotide is a modified cyclotide that binds to IGFR but has altered binding to IGFR compared to the cyclotide set forth in SEQ ID NO: 81 or 82. In some embodiments, the modified cyclotide has altered binding to IGFR compared to the cyclotide set forth in SEQ ID NOS: 82. In some embodiments, the binding affinity to the IGFR is reduced, such as reduced by 1.5-fold, 2.0-fold, 3.0-fold, 4.0-fold, 5.0-fold, 10.0-fold or more. In some embodiments, the binding affinity to IGFR is pH-dependent, such that the modified cyclotide exhibits greater binding affinity to IGFR at neutral pH (e.g. 7.0-7.4) than acidic pH of the endosome (e.g. pH of 4.5 to 6.5, such as at or about pH 5.0). In some embodiments, the modified cyclotide has a loop with a peptide that has 1, 2, 3 or 4 amino acid substitution(s) compared to the peptide set forth in SEQ ID NO: 35 or 36. In some embodiments, the modified cyclotide is modified by loop replacement (e.g. loop 1) of Mcoti-II (SEQ ID NO: 2) with a peptide that has 1, 2, 3 or 4 amino acid substitution(s) compared to the peptide set forth in any one of SEQ ID NOS: 35 or 36. In some embodiments, the amino acid substitution(s) is to a histidine. In some embodiments, the amino acid substitution(s) is to an alanine. In some embodiments, such modified cyclotides exhibit enhanced blood-brain barrier translocation characteristics.

In some embodiments, the modified cyclotides are derived from loop replacement libraries based on Mcoti-II (SEQ ID NO: 2) and bind to RAGE (e.g. human RAGE). In some embodiments, the loop replacement is replacement of at least one loop, such as loop 1, with a peptide that comprises the sequence set forth in SEQ ID NO: 37 or 38. In some embodiments, the peptide is the peptide set forth in SEQ ID NO: 37 or 38. In some embodiments, the modified cyclotide comprises the sequence set forth in SEQ ID NO: 83 or 84. In some embodiments, the modified cyclotide is set forth in SEQ ID NOS: 83 or 84. In some embodiments, the modified cyclotide is set forth in SEQ ID NO: 83. In some embodiments, the modified cyclotide is set forth in SEQ ID NO:84.

In some embodiments, the modified cyclotide is a modified cyclotie that bnds to RAGE but has altered binding to RAGE compared to the cyclotide set forth in SEQ ID NOS: 83 or 84. In some embodiments, the binding affinity to the RAGE is reduced, such as reduced by 1.5-fold, 2.0-fold, 3.0-fold, 4.0-fold, 5.0-fold, 10.0-fold or more. In some embodiments, the binding affinity to RAGE is pH-dependent, such that the modified cyclotide exhibits greater binding affinity to ErB3 at neutral pH (e.g. 7.0-7.4) than acidic pH of the endosome (e.g. pH of 4.5 to 6.5, such as at or about pH 5.0). In some embodiments, the modified cyclotide has a loop with a peptide that has 1, 2, 3 or 4 amino acid substitution(s) compared to the peptide set forth in SEQ ID NO:37 or 38. In some embodiments, the modified cyclotide is modified by loop replacement (e.g. loop 1) of Mcoti-II (SEQ ID NO: 2) with a peptide that has 1, 2, 3 or 4 amino acid substitution(s) compared to the peptide set forth in any one of SEQ ID NOS: 37 or 38. In some embodiments, the amino acid substitution(s) is to a histidine. In some embodiments, the amino acid substitution(s) is to an alanine. In some embodiments, such modified cyclotides exhibit enhanced blood-brain barrier translocation characteristics.

In some embodiments, the modified cyclotides are derived from loop replacement libraries based on Mcoti-II (SEQ ID NO: 2) and bind to the LRP-1 (e.g. human LRP-1). In some embodiments, the loop replacement is replacement of at least one loop, such as loop 1, with a peptide that comprises the sequence set forth in any one of SEQ ID NOS: 39-48. In some embodiments, the peptide is the peptide set forth in any one of SEQ ID NOS: 39-48. In some embodiments, the modified cyclotide comprises the sequence set forth in any one of SEQ ID NOS: 85-94. In some embodiments, the modified cyclotide is set forth in any one of SEQ ID NOS: 85-94. In some embodiments, the modified cyclotide comprises the sequence set forth in SEQ ID NO: 87. In some embodiments, the modified cyclotide is set forth in SEQ ID NO: 87. In some embodiments, the modified cyclotide comprises the sequence set forth in SEQ ID NO: 89. In some embodiments, the modified cyclotide is set forth in SEQ ID NO: 89.

In some embodiments, the modified cyclotide is a modified cyclotide that binds to LRP-1 but has altered binding to LRP-1 compared to the cyclotide set forth in any one of SEQ ID NOS: 85-94. In some embodiments, the binding affinity to LRP-1 is reduced, such as reduced by 1.5-fold, 2.0-fold, 3.0-fold, 4.0-fold, 5.0-fold, 10.0-fold or more. In some embodiments, the binding affinity to the leptin receptor is pH-dependent, such that the modified cyclotide exhibits greater binding affinity to LRP-1 at neutral pH (e.g. 7.0-7.4) than acidic pH of the endosome (e.g. pH of 4.5 to 6.5, such as at or about pH 5.0). In some embodiments, the modified cyclotide has a loop with a peptide that has 1, 2, 3 or 4 amino acid substitution(s) compared to the peptide set forth in any one of SEQ ID NOS: 39-48. In some embodiments, the modified cyclotide is modified by loop replacement (e.g. loop 1) of Mcoti-II (SEQ ID NO: 2) with a peptide that has 1, 2, 3 or 4 amino acid substitution(s) compared to the peptide set forth in any one of SEQ ID NOS: 39-48. In some embodiments, the amino acid substitution(s) is to a histidine. In some embodiments, the amino acid substitution(s) is to an alanine. In some embodiments, such modified cyclotides exhibit enhanced blood-brain barrier translocation characteristics.

In some embodiments, any of the provided modified cyclotides bind to a BBB-R for which the peptide targets. Methods for determining binding affinity, or relative binding affinity, are known in art, solid-phase ELISA immunoassays, ForteBio Octet, Biacore measurements or flow cytometry. See, for example, Larsen et al., American Journal of Transplantation, vol. 5: 443-453 (2005); Linsley et al., Immunity, Vol 1 (9): 793-801 (1994). In some embodiments, binding affinity can be measured by flow cytometry, such as based on a Mean Fluorescence Intensity (MFI) in a flow binding assay. In some embodiments, the provided conjugate, such as fusion protein, has a binding affinity for a BBB-R as determined by, for example, solid-phase ELISA immunoassays, flow cytometry or surface plasmon resonance (Biacore) assays.

In some embodiments, the conjugate (e.g. fusion protein) has a binding affinity for a BBB-R of greater than 10 nM and less than 1000 nM. In some embodiments, the conjugate has a binding affinity for a BBB-R of greater than 20 nM and less than 800 nM. In some embodiments, the conjugate has a binding affinity for a BBB-R of about 40 nM, 50 nM, 60 nM, 70 nM, 80 nM, 100 nM, 150 nM, 200 nM, 250 nM, 300 nM, 350 nM, 400 nM, 450 nM, 500 nM, 550 nM, 600 nM, 650 nM, 700 nM, 750 nM or any value between any of the foregoing. In some embodiments, the conjugate has a binding affinity for a BBB-R of from about 50 nM to about 500 nM. In some embodiments, the conjugate has a binding affinity for a BBB-R of from about 50 nM to about 250 nM, In some embodiments, the conjugate has a binding affinity for a BBB-R of from about 50 nM to about 100 nM. In some embodiments, the conjugate has a binding affinity for a BBB-R of from about 100 nM to about 500 nM. In some embodiments, the conjugate has a binding affinity for a BBB-R of from about 100 nM to about 250 nM. In some embodiments, the conjugate has a binding affinity for a BBB-R of from about 250 nM to 500 nM.

The cyclotide molecules provided herein may comprise a specific functionality which typically includes at least some type of membrane translocation activity. In a specific embodiment of the invention, the membrane translocation activity comprises an activity selected from one or more of: an ability to translocate across the gut wall (e.g. intestinal mucosa); an ability to translocate across the blood brain barrier; an ability to translocate across a cell membrane; an ability to translocate into a sub cellular compartment; an ability to translocate into the nucleus of a cell; and an ability to translocate into an organelle, including a mitochondrion. In some embodiments, the provided modified cyclotide exhibits improved or enhanced transcytosis across the blood brain barrier compared to the parental or scaffold cyclotide, such as the cyclotide Mcoti-II (SEQ ID NO:2). In some embodiments, the improved or enhanced trancytosis is due to binding to a receptor involved in blood-brain barrier transcytosis. The membrane translocation activity may be suitably comprised within a peptide or polypeptide that resides within the functional module a larger protein. Hence, the provided cyclotide represent synthetic polypeptide scaffold. Desirable physical properties of potential scaffold molecules include high thermal stability and reversibility of thermal folding and unfolding.

### II. CONJUGATES AND FUSION PROTEINS OF A CYCLOTIDE AND BIOLOGICAL AGENT

Also provided herein in some embodiments is a conjugate comprising: the modified cyclotide or the binding molecule of any of the preceding embodiments, and a biologically active agent. In some embodiments, the modified cyclotide or binding molecule is any as described in Section I.B. In some embodiments, the biologically active agent is a small molecule, a peptide or a protein. In some embodiments, the biologically active agent is a diagnostic agent or a therapeutic agent.

In some embodiments, the biologically active agent is any agent that has therapeutic potential for treatment of pathology in the central nervous system (CNS). In some embodiments, the biologically active agent can be selected from neurological disorder drugs, neurotrophic factors, growth factors, enzymes, cytotoxic agents, antibodies directed to a brain target, monoclonal antibodies directed to a brain target, or peptides directed to a brain target.

A cyclotide of the invention may also be conjugated or otherwise linked to a biologically active agent or therapeutic selected from the group consisting of: a small molecule; an antibody or antibody fragment; a hormone, a cytokine; a nucleic acid; a bioactive peptide; a glycosylated peptide; an imaging agent; and a radioactive compound.

In some embodiments, the cyclotide moiety can be an amino acid extension of the C- or N-terminus of the biological agent, e.g. polypeptide. In some aspects, a short amino acid linker sequence may lie between the biological agent, e.g. polypeptide, and the cyclotide moiety. Suitable linker groups may comprise an amide, an ester, a disulfide, a sulfide, a ketal, a succinate, an oxime, a carbamate, a carbonate, a sialyl ether, or a triazole. In some embodiments, a linker can be a peptide linker. In some embodiments, provided molecules include those where the biological agent, e.g. polypeptide, is linked, e.g. by chemical conjugation to the cyclotide moiety, optionally via a linker sequence. Typically, the modified polypeptide will be linked to the other moiety via sites that do not interfere with the activity of either moiety.

In some of any of the preceding embodiments, the conjugate is a fusion protein comprising the modified cyclotide operably linked to a biologically active agent that is a protein, polypeptide or peptide.

In some of any of the preceding embodiments, the biologically active agent is selected from the group consisting of an antibody or an antibody fragment thereof. In some of any of the preceding embodiments, the biologically active agent is selected from the group consisting of a growth factor or a hormone. In some of any of the preceding embodiments, the biologically active agent is an enzyme.

In other embodiments of the invention, cyclotides are conjugated to or comprised within desirable biological therapeutic agents which are then administered to a test animal in order to determine the BBB transfer of the modified biological therapeutic agent.

Therapeutic uses and applications for the cyclotides of the invention therefore include any disease or condition that requires repetitive treatment regimes or the frequent administration of a biologically active agent. This includes, therapeutic applications that may benefit from conjugation of a cyclotide to the N- or C-terminus of the therapeutic agent that so renders the therapeutic agent more readily available to the brain or cerebrospinal fluid of the recipient. Diseases that are suitable for treatment include but are not limited to: the treatment of various neoplastic and non-neoplastic diseases and disorders (e.g. cancers/neoplastic diseases and related conditions); chronic degenerative and neurodegenerative diseases or disorders (e.g. multiple sclerosis, Parkinson's disease and Alzheimer's disease), strokes or other brain damaging conditions. In a further embodiment, cyclotides of the invention are fused to a therapeutic agent and used as an intravenously administered therapeutic treatment of Alzheimer's disease or intracranial neoplasms. In a further embodiment, a cyclotide of the invention is conjugated to a therapeutic agent capable of enhancing cognitive ability such as memory.

In a further aspect, the present invention relates to a conjugate which may comprise a carrier selected from the group consisting of any one of the modified cyclotides of the present invention linked to a bioactive or therapeutic agent selected from the group consisting, for example, of a drug (e.g., a small molecule drug, e.g., an antibiotic), a medicine, a detectable label, a protein (e.g., an enzyme), protein-based compound (e.g., a protein complex comprising one or polypeptide chain) and a polypeptide (peptide). The agent may be more particularly, a molecule which is active at the level of the central nervous system. The agent may be any agent for treating or detecting a neurological disease.

In accordance with the present invention, the detectable label may be a radio imaging agent. Example of a label which may be conjugated with the carrier of the present invention and which is encompassed herein includes, for example and without limitation, an isotope, a fluorescent label (e.g., rhodamine), a reporter molecule (e.g., biotin), etc. Other examples of detectable labels include, for example, a green fluorescent protein, biotin, a his tag protein and beta-galactosidase.

In some embodiments, the biologically active agent is a therapeutic protein agent. Example of therapeutic protein or protein-based compound which may be conjugated with the carrier of the present invention and which is encompassed herein includes, without limitation, an antibody, an antibody fragment (e.g., an antibody binding fragment such as a Fv fragment, F(ab)2, F(ab)2′ and Fab and the like), a single domain antibody (e.g. such as a camelid VHH domain, a shark novel antigen receptor (NAR), or a human VH or VL domain), a peptidic- or protein-based drug (e.g., a positive pharmacological modulator (agonist) or an pharmacological inhibitor (antagonist)) etc. Other examples of agent which are encompassed herein include growth factors (e.g. Fibroblast Growth Factor and related proteins, Nerve Growth Factor, Glial Cell-derived Neurotrophic Factor, Brain-Derived Neurotrophic Factor, Neurotrophin-3 and Neurotrophin-4), cellular toxins (e.g., monomethyl auristatin E (MMAE), toxins from bacteria endotoxins and exotoxins; diphtheria toxins, botunilum toxins, tetanus toxins, pertussis toxins, staphylococcus enterotoxins, toxic shock syndrome toxin TSST-1, adenylate cyclase toxin, shiga toxin, cholera enterotoxin, and others), soluble receptors (such as TNF receptor 1 or 2) and anti-angiogenic compounds (endostatin, catechins, nutriceuticals, chemokine IP-10, inhibitors of matrix metalloproteinase (MMPIs), anastellin, vironectin, antithrombin, tyrosine kinase inhibitors, VEGF inhibitors, antibodies against receptor, Herceptin®, avastin and panitumumab and others).

In some embodiments, the biologically active agent is a neurological disorder therapeutic agent. In some embodiments, a neurological disorder therapeutic agent includes, but are not limited to, small molecule compounds, antibodies, peptides, proteins, natural ligands of one or more CNS target(s), modified versions of natural ligands of one or more CNS target(s), aptamers, inhibitory nucleic acids (i.e., small inhibitory RNAs (siRNA) and short hairpin RNAs (shRNA)), ribozymes, and small molecules, or active fragments of any of the foregoing. Exemplary neurological disorder drugs of the invention are described herein and include, but are not limited to: antibodies, aptamers, proteins, peptides, inhibitory nucleic acids and small molecules and active fragments of any of the foregoing that either are themselves or specifically recognize and/or act upon (i.e., inhibit, activate, or detect) a CNS antigen or target molecule. In some embodiments, a CNS antigen target molecules includes, but are not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins. Non-limiting examples of neurological disorder drugs and the corresponding disorders they may be used to treat include brain-derived neurotrophic factor (BDNF) for treating chronic brain injury (Neurogenesis); anti-EGFR antibody for treating Brain cancer; Glial cell-line derived neural factor (GDNF) for treating Parkinson's disease; Brain-derived neurotrophic factor (BDNF) for treating Amyotrophic lateral sclerosis or depression; Lysosomal enzyme for treating Lysosomal storage disorders of the brain; Ciliary neurotrophic factor (CNTF) for treating Amyotrophic lateral sclerosis; Neuregulin-1 for treating Schizophrenia; Anti-HER2 antibody (e.g. trastuzumab) for treating brain metastasis from HER2-positive cancer.

In some embodiments, the biologically active agent is an antibody. In some embodiments the antibody is a therapeutic antibody. In some embodiments, the antibody is a full length antibody directed to a brain target. In some embodiments, the antibody is a full length IgG. Previous studies have illustrated that a very small percentage (approximately 0.1%) of an IgG injected in the bloodstream are able to penetrate into the CNS compartment (Felgenhauer, Klin. Wschr. 52: 1158-1164 (1974)). The provided embodiments thus provide for improved therapeutics with enhanced blood-brain barrier translocation activity.

In some embodiments, the antibody is directed against, such as specifically binds, an antigen and/or molecule expressed in the CNS, including the brain, which can be targeted with an antibody or small molecule. Examples of such antigen and/or molecule include, without limitation: beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6.

In some embodiments, the antibody is directed against or binds HER2. In some embodiments, the antibody is trastuzumab (Herceptin®). In some embodiments the antibody contains a heavy chain sequence that exhibits at least about 85%, at least about 90%, or at least about 95% sequence identity to the heavy chain sequence set forth in SEQ ID NO:134 and a light chain sequence that exhibits at least about 85%, at least about 90%, or at least about 95% sequence identity to the light chain sequence set forth in SEQ ID NO:137. In some embodiments, the antibody contains the heavy chain sequence set forth in SEQ ID NO:134 and the light chain sequence set forth in SEQ ID NO:137. In some embodiments, antibodies targeting HER2, and provided conjugate fusions containing same, can be used for treating metastatic breast cancer. In some embodiments, antibodies targeting HER2, and provided conjugate fusions containing same, can be used for treating a neurological disease, such as Alzheimer's Disease.

In some embodiments, the antibody is directed against or binds amyloid beta. In some embodiments, the antibody is aducanumab (Aduhelm). In some embodiments the antibody contains a heavy chain sequence that exhibits at least about 85%, at least about 90%, or at least about 95% sequence identity to the heavy chain sequence set forth in SEQ ID NO:129 and a light chain sequence that exhibits at least about 85%, at least about 90%, or at least about 95% sequence identity to the light chain sequence set forth in SEQ ID NO:132. In some embodiments, the antibody contains the heavy chain sequence set forth in SEQ ID NO:129 and the light chain sequence set forth in SEQ ID NO:132. In some embodiments, antibodies targeting amyloid beta, and provided conjugate fusions containing same, can be used for treating Alzheimer's Disease.

In some embodiments, the biologically active agent is a TNF inhibitor. In some embodiments, the inhibitor is a small molecule, peptide or protein. Ins ome embodiments, the inhibitor is an antibody. In some embodiments, the antibody is directed against or binds TNF-alpha. In some embodiments, the antibody is adalimumab (Humira). In some embodiments the antibody contains a heavy chain sequence that exhibits at least about 85%, at least about 90%, or at least about 95% sequence identity to the heavy chain sequence set forth in SEQ ID NO:102 and a light chain sequence that exhibits at least about 85%, at least about 90%, or at least about 95% sequence identity to the light chain sequence set forth in SEQ ID NO:103. In some embodiments, the antibody contains the heavy chain sequence set forth in SEQ ID NO:102 and the light chain sequence set forth in SEQ ID NO:103. In some embodiments, antibodies targeting TNF-alpha, and provided conjugate fusions containing same, can be used for treating a neurological disease, such stroke, traumatic brain injury or Alzheimer's Disease.

In some embodiments, the biologically active agent is a growth factor or a hormone. In some embodiments, the biologically active agent is a growth factor. Cyclotides of the invention may be operably linked to growth factors including ADNP, BDNF, CNTF, GCSF, GDNF, IFN Beta, IL-10, Insulin, MANF, NGF, NT-3, Progranulin, amongst others in order to treat a variety of neurological disorders such stroke, Alzheimers, Parkinsons disease and multiple sclerosis. In some embodiments, the biologically active agent is a nerve growth factor (NGF), such as for treating Parkinson's Disease, stroke or Alzheimer's Disease. In some embodiments, the biologically active agent is granulocyte colony-stimulating factor (GCSF), such as for using in treating stroke or traumatic brain injury (TBI). In some embodiments, the biologically active agent is IL-10 such as for use in treating Alzheimer's Disease, heart disease or brain cancer. In some embodiments, the biologically active agent is BDNF, such as for use in treating ALS or depression. In some embodiments, the biologically active agent is ADNP, such as for use in treating Autism like disorder.

In some embodiments, the biologically active agent is a NGF and has the sequence set forth in SEQ ID NO:147 or a sequence that exhibits at least about 85%, at least about 90% or at least about 95% sequence identity to SEQ ID NO:147. In some embodiments, the biologically active agent is set forth in SEQ ID NO:147.

In some embodiments, the biologically active agent is an enzyme. In some embodiments, the enzyme is a ceramide degrading enzyme, a lipase, a hydrolase type enzyme or a sulfatase. In some embodiments, the enzyme is a ceramide degrading enzyme and the ceramide degrading enzyme is glucocerebrosidase, galactocerebrosidase or alpha galactosidase. In some embodiments, the enzyme is a lipase or a hydrolase type enzyme and the enzyme is sphinomyelinase, cerliponase or alpha glucosidase. In some embodiments, the enzyme is heparin N Sulfatase, such as for treating Sanfilippo A. In some embodiments, the enzyme is glucocerebrosidase, such as for treating Gaucher's Disease. In some embodiments, the enzyme is glucocerebrosidase, such as for treating Parkinson's Disease. In some embodiments, the enzyme is galactocerebrosidase, such as for use in treating Krabbe's disease. In some embodiments, the enzyme is alpha galactosidase, such as for use in treating Fabry's disease. In some embodiments, the enzyme is sphinomyelinase, such as for treating Niemann Pick. In some embodiments, the enzyme is cerliponase alpha, such as for use in treating Jansky Bielschowsky. In some embodiments, the enzyme is alpha glucosidase, such as for treating Pompe's. In some embodiments, the enzyme is tripeptidyl peptidase I, such as for using in treating Jansky Bieschowsky or Batten. In some embodiments, the enzyme is galactosamine 6 sulfatase, such as for use in treating Marquio.

In some embodiments, the biologically active agent is glucocerebrosidase (GCase). In some embodiments, the enzyme has a sequence of amino acids set forth in SEQ ID NO:144 or a sequence of amino acids that exhibits at least about 85%, at least about 90% or at least about 95% sequence identity to SEQ ID NO:144. In some embodiments, the sequence is set forth in SEQ ID NO:144. In some embodiments, the enzyme has a sequence of amino acids set forth in SEQ ID NO:145 or a sequence of amino acids that exhibits at least about 85%, at least about 90% or at least about 95% sequence identity to SEQ ID NO:145. In some embodiments, the sequence is set forth in SEQ ID NO:145. In some embodiments, the glucocerebrosidase is a glucocerebrosidase mutant GCase molecule that exhibits increased stability and enhanced function. An exemplary GCase mutant has one or more amino acid substitutions selected from I5N, F31Y, L34Q, M53T, P55T, H145F, H145L, H223N, H223Y, E233Q, H274N, W312C, F316A, L317F, K321V, K321A, K321N, A341C, H365K, I368C, D443C, D445C, H451K, S455C, S464C, R495C, R495N, and combinations thereof, relative to the sequence set forth in SEQ ID NO: 144 or SEQ ID NO:145. In some embodiments, the GCase mutant contains 1, 2, 3, 4, 5, 6 or 7 amino acid substitutions from I5N, F31Y, L34Q, M53T, P55T, H145F, H145L, H223N, H223Y, E233Q, H274N, W312C, F316A, L317F, K321V, K321A, K321N, A341C, H365K, I368C, D443C, D445C, H451K, S455C, S464C, R495C, R495N, relative to the sequence set forth in SEQ ID NO: 144 or SEQ ID NO:145. In some embodiments, the GCase mutant has the amino acid substitutions M53T and P55T, relative to the sequence set forth in SEQ ID NO:144 or SEQ ID NO:145. In some embodiments, the GCase mutant has the amino acid substitutions H145L/K321N, D443C/S464C, S455C/R495C, W312C/A341C, I368C/D445C, and E233Q/W312C/A341C, relative to the sequence set forth in SEQ ID NO: 144 or SEQ ID NO:145. In some embodiments, the GCase mutant has the amino acid substitutions F316A/L317F, F316A/L317F/K321N, or H145L/K321N, relative to the sequence set forth in SEQ ID NO: 144 or SEQ ID NO:145. Non-limiting examples of GCase mutants are described in published PCT Appl. No. WO2012/064709; WO2021/048034; and WO2022/023761.

Among the provided conjugates is a modified cyclotide linked to a glucocerebrosidase. In some embodiments, the modified cyclotide is set forth in SEQ ID NO:101, such as for targeting TrfR. In some embodiments, the modified cyclotide is set forth in SEQ ID NO:81, such as for targeting IgFR. In some embodiments, the modified cyclotide is set forth in SEQ ID NO:82, such as for targeting IgFR. In some embodiments, the modified cyclotide is set forth in SEQ ID NO:85, such as for targeting LRP-1. In some embodiments, the modified cyclotide is set forth in SEQ ID NO:87, such as for targeting LRP-1. In some embodiments, the modified cyclotide is set forth in SEQ ID NO:89, such as for targeting LRP-1. In some embodiments, the modified cyclotide is set forth in SEQ ID NO:83, such as for targeting RAGE. In some embodiments, the modified cyclotide is set forth in SEQ ID NO:84, such as for targeting RAGE. In some embodiments, the modified cyclotide targets TrfR and the conjugate has the sequence of amino acids set forth in SEQ ID NO:146 or a sequence of amino acids that has at least 90%, 91%, 92%, 93%, 94%, 05%, 96%, 97%, 98%, 99% or more sequence identity to SEQ ID NO:146. In some embodiments, the conjugate has the sequence set forth in SEQ ID NO:146. In some embodiment, the modified cyclotide binds to a BBB-R (e.g, TrfR) to transport the glucocerebrosidase across the blood brain barrier.

In some embodiments, the conjugate, such as a fusion protein of a modified cyclotide and a biological protein agent, contains 1, 2, 3, 4, 5 or more copies of the modified cyclotide. In some embodiments, the conjugate, such as a fusion protein as provided, contains one modified cyclotide such that the conjugate (e.g. fusion protein) exhibits monovalent for binding the BBB-R. In particular embodiments, the conjugate (e.g. fusion protein) is monovalent for binding any one BBB-R.

In some embodiments, the conjugate (e.g. fusion protein) may contain a plurality of copies of the modified cyclotide, such as 2, 3, 4 or 5 copies of the modified cyclotide. In such an embodiment, the conjugates (e.g. fusion proteins) may be referred to as a multivalent binding protein. Also provided is a multivalent binding protein comprising a plurality of any of the provided modified cyclotides. In some embodiments, each of the modified cyclotides may be linked, directly or indirectly, in tandem on the same polypeptide chain. In some embodiments, the fusion protein is a multichain polypeptide and each chain of the polypeptide contains at least one copy of a modified cyclotide. For instance, in some embodiments, there is provided a fusion protein of an antibody (e.g. therapeutic antibody) and a modified cyclotide in which a first heavy chain or light chain of the antibody is linked to a first copy of the modified cyclotide and the second heavy chain or light chain is linked to a second copy of the modified cyclotide. In some embodiments, the binding protein is bivalent and contains two copies of the modified cyclotide.

In some embodiments, the conjugate (e.g. fusion protein) may contain a plurality of different modified cyclotides, such as 2, 3, 4 or 5 different modified cyclotides. In such an embodiment, the conjugates (e.g. fusion proteins) may be referred to as a multispecific binding protein. Also provided is a multispecific binding protein comprising a plurality of different cyclotides from any of the provided modified cyclotides. In some embodiments, the binding protein is multispecific and contains at least two different modified cyclotides that each bind to a different BBB-R. In some embodiment, the multispecific binding protein contains 2, 3 or 4 different modified cyclotides in which each modified cyclotides binds a different BBB-R. In some embodiments, each of the modified cyclotides may be linked, directly or indirectly, in tandem on the same polypeptide chain. In some embodiments, the fusion protein is a multichain polypeptide and each chain of the polypeptide contains a modified cyclotide, which can be different. For instance, in some embodiments, there is provided a fusion protein of an antibody (e.g. therapeutic antibody) and a modified cyclotide in which a first heavy chain or light chain of the antibody is linked to a first modified cyclotide and the second heavy chain or light chain is linked to a second modified cyclotide that is different from the first modified cyclotide. In some embodiments, the binding protein is bispecific and contains two different modified cyclotides that each bind to a different BBB-R. As an example, in some embodiments, a first modified cyclotide binds to the transferrin receptor and the second modified cyclotide binds to the IGF1R.

A plurality modified cyclotides (the same or different) need not be covalently linked directly to one another. In some embodiments, an intervening span of one or more amino acid residues indirectly covalently bonds the modified cyclotides (e.g. first modified cyclotide and second modified cyclotide) to each other. The linkage can be via the N-terminal to C-terminal residues. In some embodiments, the linkage can be made via side chains of amino acid residues that are not located at the N-terminus or C-terminus modified cyclotide. Thus, linkages can be made via terminal or internal amino acid residues or combinations thereof.

In some embodiments, any of a plurality of modified cyclotides (the same or different) can be to a biological agent (or a chain thereof) separated with a peptide linker, such as a flexible linker. In some embodiments, the peptide linker may be GGGS or other similar flexible linker, including longer linkers of (GGGS)n where n is 1-3. In some embodiments, the conjugate (e.g. fusion protein) containing a plurality of modified cyclotides may include 2, 3, 4 or more modified cyclotides. In some embodiments, the modified cyclotides may be the same or different. In some embodiments, the modified cycltoides may be the same to produce a bivalent, trivalent or other multivalent molecule. In some embodiments, the modified cyclotides may be different to provide a bispecific, trispecific or other multispecific molecule.

In some embodiments, any of a plurality of modified cyclotides (the same or different) can be linked in tandem with a biological agent in a single polypeptide chain separated with a peptide linker, such as a flexible linker. In some embodiments, the peptide linker may be GGGS or other similar flexible linker, including longer linkers of (GGGS)n where n is 1-3. In some embodiments, the tandem single polypeptide may include 2, 3, 4 or more modified cyclotides to produce a bivalent, trivalent, tetravalent or other multivalent molecule. In some embodiments, the one or more modified cyclotides may be the same or different. In some embodiments, the tandem singly polypeptide may include 2, 3, 4 or more different cyclotides to provide a bispecific, trispecific or other multispecific molecule.

In some embodiments, the fusion protein is composed of an antibody (e.g. therapeutic antibody) and at least one modified cyclotide. In some embodiments, the antibody can be linked to 1, 2, 3 or 4 modified cyclotides, any of which may be the same or different. In some embodiments, the modified cyclotides can be linked to the heavy chain or the light chain of the antibody. In some embodiments, the modified cyclotides can be linked to the N-terminus of a heavy chain or a light chain the antibody. In some embodiments, the modified cyclotides can be linked to the C-terminus of a heavy chain or a light chain of the antibody. In some embodiments, each of the modified cyclotides are linked to the heavy chain of the antibody. In some embodiments, each chain of the antibody is linked to a modified cyclotide.

In some embodiments, there is provided a bivalent fusion protein composed of an antibody (e.g. a therapeutic antibody) and two copies of a modified cyclotide. In some embodiments, the modified cyclotide is linked to both heavy chains of the antibody. In some embodiments, the modified cyclotide is linked to both light chains of the antibody. In some embodiments, the modified cyclotides can be linked to the N-terminus of a heavy chain or a light chain the antibody. In some embodiments, the modified cyclotides can be linked to the C-terminus of a heavy chain or a light chain of the antibody. Methods of making antibody fusions are known. In some embodiments, the heavy chain and light chain are co-expressed in a cell. In some embodiment, when produced in a cell, a two chain polypeptide is formed by dimerization resulting from disulfide formation between two heavy chain molecules. In some embodiments, the antibody fusion protein is a homodimer containing two identical copies of the modified cyclotide.

In some embodiment, there is provided a monovalent fusion protein composed of antibody in which there is linked a single modified cyclotide. In such an embodiments, the antibody fusion protein is monovalent for binding the BBB-R. In some embodiments, the modified cyclotide is linked to one heavy chain of the antibody. In some embodiments, the modified cyclotide is linked to one light chain of the antibody. In some embodiments, the modified cyclotides can be linked to the N-terminus of a heavy chain or a light chain the antibody. In some embodiments, the modified cyclotides can be linked to the C-terminus of a heavy chain or a light chain of the antibody. In some embodiments, the fusion protein is a heterodimer composed of two different heavy chains and a light chain that pairs with each heavy chain, in which only one of the heavy chains is linked to the modified cyclotide.

In some embodiment, there is provided a multispecific fusion protein composed of an antibody in which there is linked at least two different modified cyclotides. In some embodiments, the antibody fusion protein is bispecific and contains two different modified cyclotides for binding two different BBB-R. In some embodiments, each modified cyclotide is linked to a heavy chain of the antibody. In some embodiments, the modified cyclotides may be linked the a heavy chain in tandem. In some embodiments, the modified cyclotides may each be linked to a different heavy chain of the antibody. In some embodiments, each modified cyclotide is linked to a light chain of the antibody. In some embodiments, the modified cyclotides may be linked the a light chain in tandem. In some embodiments, the modified cyclotides may each be linked to a different light chain of the antibody. In some embodiments, the modified cyclotides can be linked to the N-terminus of a heavy chain or a light chain the antibody. In some embodiments, the modified cyclotides can be linked to the C-terminus of a heavy chain or a light chain of the antibody. In some embodiments, the fusion protein is a heterodimer composed of two different heavy chains and a light chain that pairs with each heavy chain, in which one of the heavy chains is linked to different modified cyclotide in tandem and the other heavy chain is not linked to a modified cyclotide. In some embodiments, the fusion protein is a heterodimer composed of two different heavy chains and a light chain that pairs with each heavy chain, in which each of the heavy chains is linked to a different modified cyclotide.

Methods for producing heterodimeric antibody fusion proteins are known. In some embodiments, two different heavy chains may be co-expressed in a cell using knobs-into-hole engineering strategy or other strategy to produce a heterodimer in which two different heavy chains, for example each carrying a different modified cyclotide, may interact to form a heterodimer. In some embodiments, residues of the constant chain are modified by amino acid substitution to promote the heterodimer formation. In some of any embodiments, the one more amino acid modifications are selected from a knob-into-hole modification and a charge mutation to reduce or prevent self-association due to charge repulsion. The heterodimer can be formed by transforming into a cell both a first nucleic acid molecule encoding a first heavy chain polypeptide subunit (e.g. knob sequence) and a second nucleic acid molecule encoding a second different heavy chain polypeptide subunit (e.g. hole sequence). In some aspects, the heterodimer is produced upon expression and secretion from a cell as a result of covalent or non-covalent interaction between residues of the two polypeptide subunits to mediate formation of the dimer. In such processes, generally a mixture of dimeric molecules is formed, including homodimers and heterodimers. For the generation of heterodimers, additional steps for purification can be necessary. For example, the first and second polypeptide can be engineered to include a tag with metal chelates or other epitope, where the tags are different. The tagged domains can be used for rapid purification by metal-chelate chromatography, and/or by antibodies, to allow for detection by western blots, immunoprecipitation, or activity depletion/blocking in bioassays. In other embodiments, methods can be carried out that promote heterodimerization.

Methods include those described in U.S. Pat. No. 10,995,127. For example, having an amino acid modification within the CH3 domain at Thr366, which when replaced with a more bulky amino acid, e.g., Try (T366W), is able to preferentially pair with a second CH3 domain having amino acid modifications to less bulky amino acids at positions Thr366, Leu368, and Tyr407, e.g., Ser, Ala and Val, respectively (T366S/L368A/Y407V). In some embodiments, the “knob” Fc domain comprises the mutation T366W. In some embodiments, the “hole” Fc domain comprises mutations T366S, L368A, and Y407V. Heterodimerization via CH3 modifications can be further stabilized by the introduction of a disulfide bond, for example by changing Ser354 to Cys (S354C) and Y349 to Cys (Y349C) on opposite CH3 domains (Reviewed in Carter, 2001 Journal of Immunological Methods, 248: 7-15). In some embodiments, Fc domains used for heterodimerization comprise additional mutations, such as the mutation S354C on a first member of a heterodimeric Fc pair that forms an asymmetric disulfide with a corresponding mutation Y349C on the second member of a heterodimeric Fc pair. In some embodiments, one member of a heterodimeric Fc pair comprises the modification H435R or H435K to prevent protein A binding while maintaining FcRn binding. In some embodiments, one member of a heterodimeric Fc pair comprises the modification H435R or H435K, while the second member of the heterodimeric Fc pair is not modified at H435. In various embodiments, the hole Fc domain comprises the modification H435R or H435K (referred to as “hole-R” in some instances when the modification is H435R), while the knob Fc domain does not. In some instances, the hole-R mutation improves purification of the heterodimer over homodimeric hole Fc domains that may be present.

It is within the level of a skilled artisan to make appropriate knob and hole modifications into the heavy chain of any antibody. As an example, a knob and hole sequence of the exemplary antibody trastuzumab is set forth in SEQ ID NO:135 and SEQ ID NO:136, respectively. In another example, a knob and hole sequence of the exemplary antibody aducanumab is set forth in SEQ ID NO: 130 and SEQ ID NO:131, respectively. Any antibody can be similarly modified in the CH3 domain to create knob and hole chains to promote heterodimerization. In some embodiments, a modified cyclotide is linked to the knob heavy chain and/or a hole heavy chain of an antibody. In some embodiments, the knob heavy chain, hole heavy chain and light chain are co-expressed in a cell in which a heterodimer antibody containing two different heavy chains (knob and hole) each complexed with the light chain is produced.

In some embodiments, one or more “peptide linkers” link the modified cyclotide and one or more other modified cyclotide or the biological agent. In some embodiments, a peptide linker can be a single amino acid residue or greater in length. In some embodiments, the peptide linker has at least one amino acid residue but is no more than 20, 19, 18, 17, 16, 15, 14, 13, 12, 11, 10, 9, 8, 7, 6, 5, 4, 3, 2, or 1 amino acid residues in length. In some embodiments, the pIn some embodiments, the linker is a flexible linker. In some embodiments, the linker is (in one-letter amino acid code): GGGGS (“4GS”) or multimers of the 4GS linker, such as repeats of 2, 3, 4, or 5 4GS linkers. In some embodiments, the linker is or includes GGGGS (SEQ ID NO:148). In some embodiments, the peptide linker is or includes (GGGGS)₂ or (GGGGS)₃ as set forth in SEQ ID NOs: 154 and 155, respectively. In some embodiments, the linker also can include a series of alanine residues alone or in addition to another peptide linker (such as a 4GS linker or multimer thereof). In some embodiment, the linker is GSGGGSGGGGSGGGGS (SEQ ID NO:104).

In some embodiments, the linker is a GS linker of at least 10 amino acids in length, such as at least 15 amino acids in length. In some embodiments, the GS linker is 10, 11, 12, 13, 14, 15, 16, 17, 18, 19, 20, 21, 22, 23, 24, or 25 amino acids in length, or any value between any of the foregoing. In some embodiments the GS linker is 10 to 25 amino acids. In some embodiments the GS linker is 10 to 20 amino acids. In some embodiments, the GS linker is GGSGGSGGSGGS, i.e., (GGS)4 (SEQ ID NO: 186); GGSGGSGGSGGSGGS, i.e., (GGS)5 (SEQ ID NO: 187); GGGGGSGGGGGSGGGGGS, i.e., (G5S)3 (SEQ ID NO: 188), GGSGGGGSGGGGSGGGGS (SEQ ID NO: 189) and GGGGSGGGGSGGGGS (SEQ ID NO:155), or GSGGGSGGGGSGGGGS (SEQ ID NO:104). In some embodiments, the GS linker is set forth in SEQ ID NO:104.

In some embodiments, the linker is a cleavable linker. In some embodiments, the use of a cleavable linker can be used to ensure release of the free protein, i.e. biological agent (e.g. therapeutic agent) to the brain. Examples of cleavable linkers include the acid labile linkers. Acid labile linkers include cis-aconitic acid, cis-carboxylic alkadienes, cis-carboxylic alkatrienes, and poly-maleic anhydrides. Other cleavable linkers are linkers capable of attaching to primary alcohol groups.

In some embodiments, the linker is a cleavable linker that contains an endosome-specific protease cleavage site. In some embodiments, the endosome-specific protease cleavage site is a cathepsin cleavage site and the linker is a cathepsin cleavable linker. In some embodiments, the cathespin is a cathepsin B, a cathepsin D, a cathepsin K, a cathepsin S or a cathepsin L. In some embodiments, the cathepsin cleavage site may be any as described in PCT publication No. WO2016/050934. In some embodiments, the linker containing a cathepsin cleavage site is set forth in any one of SEQ ID NOS: 133 and 156-176. In some embodiments, the linker has a cathepsin B cleavage site. In some embodiments, the cathepsin B linker is set forth in SEQ ID NO:133.

Among the provided conjugates is a bivalent cyclotide antibody fusion composed of an antibody linked to a modified cyclotide, such as any modified cyclotide described herein. In some embodiments, the modified cyclotide is linked to the C-terminus of both heavy chains of the antibody. In some embodiments, the antibody is adalimumab. In some embodiments, the antibody fusion contains a heavy chain composed of the adalimumab heavy chain set forth in SEQ ID NO:102, a linker and a modified cyclotide, such as any described in Section I.B; and a light chain set forth in SEQ ID NO:103. In some embodiments, the antibody fusion contains a heavy chain composed of the trastuzumab heavy chain set forth in SEQ ID NO:103, a linker and a modified cyclotide, such as any described in Section I.B; and a light chain set forth in SEQ ID NO:137. In some embodiments, the antibody is a homodimer composed of two identical heavy chains and two identical light chains. In some embodiments, the modified cyclotide is set forth in SEQ ID NO:72, such as for targeting TrfR. In some embodiments, the modified cyclotide is set forth in SEQ ID NO:101, such as for targeting TrfR. In some embodiments, the modified cyclotide is set forth in SEQ ID NO:81, such as for targeting IgFR. In some embodiments, the modified cyclotide is set forth in SEQ ID NO:82, such as for targeting IgFR. In some embodiments, the modified cyclotide is set forth in SEQ ID NO:85, such as for targeting LRP-1. In some embodiments, the modified cyclotide is set forth in SEQ ID NO:87, such as for targeting LRP-1. In some embodiments, the modified cyclotide is set forth in SEQ ID NO:89, such as for targeting LRP-1. In some embodiments, the modified cyclotide is set forth in SEQ ID NO:83, such as for targeting RAGE. In some embodiments, the modified cyclotide is set forth in SEQ ID NO:84, such as for targeting RAGE. In some embodiments, the linker is a GS linker of at least 10 amino acids in length, such as at least 15 amino acids in length. In some embodiments the GS linker is 10 to 25 amino acids. In some embodiments, the GS linker is set forth in SEQ ID NO:104. Also provided herein are antibody fusion conjugates having a sequence that has at least 90%, 91%, 92%, 93%, 94%, 05%, 96%, 97%, 98%, 99% or more sequence identity to any of the foregoing amino acid sequences. In some embodiment, the modified cyclotide binds to the BBB-R recognized by the modified cyclotide to to transport the antibody across the blood brain barrier.

Among the provided conjugates is a monovalent cyclotide antibody fusion composed of an antibody linked to a modified cyclotide, such as any modified cyclotide described herein. In some embodiments, the modified cyclotide is linked to the C-terminus of one heavy chain of a heterodimeric antibody. In some embodiments, the antibody fusion contains a knob heavy chain of the antibody; a second heavy chain composed of a hole heavy chain of the antibody; and a light chain of the antibody, in which either the knob heavy chain or the hole heavy chain is linked at its C-terminus by a linker to a modified cyclotide, such as any described in Section I.B. The antibody can be a heterodimeric antibody (e.g., knob and hole variant) of any desired therapeutic or diagnostic antibody desired for transport across the blood-brain barrier. Non-limiting examples of antibodies include any as described herein. In one embodiment, the antibody is aducanumab. In some embodiments, the antibody fusion contains a first heavy chain that is a hole aducanumab heavy chain set forth in SEQ ID NO:131; a second heavy chain composed of a knob aducanumab heavy chain set forth in SEQ ID NO: 130, a linker and a modified cyclotide, such as any described in Section I.B; and a light chain set forth in SEQ ID NO:132. In another embodiment, the antibody fusion contains a first heavy chain composed of a aducanumab hole heavy chain set forth in SEQ ID NO:131, a linker, and a modified cyclotide, such as any described in Section I.B; a second heavy chain that is a knob aducanumab heavy chain set forth in SEQ ID NO: 130; and a light chain set forth in SEQ ID NO:132. In a further embodiment, the antibody fusion contains a first heavy chain that is a trastuzumab hole heavy chain set forth in SEQ ID NO:136 or 178; a second heavy chain composed of a knob trastuzumab heavy chain set forth in SEQ ID NO: 135, a linker and a modified cyclotide, such as any described in Section I.B; and a light chain set forth in SEQ ID NO:137. In some embodiments, the modified cyclotide is set forth in SEQ ID NO:72, such as for targeting TrfR. In some embodiments, the modified cyclotide is set forth in SEQ ID NO:101, such as for targeting TrfR. In some embodiments, the modified cyclotide is set forth in SEQ ID NO:81, such as for targeting IgFR. In some embodiments, the modified cyclotide is set forth in SEQ ID NO:82, such as for targeting IgFR. In some embodiments, the modified cyclotide is set forth in SEQ ID NO:85, such as for targeting LRP-1. In some embodiments, the modified cyclotide is set forth in SEQ ID NO:87, such as for targeting LRP-1. In some embodiments, the modified cyclotide is set forth in SEQ ID NO:89, such as for targeting LRP-1. In some embodiments, the modified cyclotide is set forth in SEQ ID NO:83, such as for targeting RAGE. In some embodiments, the modified cyclotide is set forth in SEQ ID NO:84, such as for targeting RAGE. In some embodiments, the linker is a GS linker of at least 10 amino acids in length, such as at least 15 amino acids in length. In some embodiments the GS linker is 10 to 25 amino acids. In some embodiments, the GS linker is set forth in SEQ ID NO:104. Also provided herein are antibody fusion conjugates having a sequence that has at least 90%, 91%, 92%, 93%, 94%, 05%, 96%, 97%, 98%, 99% or more sequence identity to any of the foregoing amino acid sequences. In some embodiment, the modified cyclotide binds to the BBB-R recognized by the modified cyclotide to to transport the antibody across the blood brain barrier.

Among the provided conjugates is a bispecific cyclotide antibody fusion composed of an antibody linked to two different modified cyclotides, such as any modified cyclotide described herein. In some embodiments, one modified cyclotide is linked to the C-terminus of one heavy chain of a heterodimeric antibody and the other modified cyclotide is linked to the C-terminus of the other heavy chain of the heterodimeric antibody. In some embodiments, the antibody fusion contains a knob heavy chain of the antibody linked at its C-terminus by a linker to a first modified cyclotide; a second heavy chain composed of a hole heavy chain of the antibody linked at its C-terminus by a linker to a second modified cyclotide; and a light chain of the antibody. The antibody can be a heterodimeric antibody (e.g., knob and hole variant) of any desired therapeutic or diagnostic antibody desired for transport across the blood-brain barrier. Non-limiting examples of antibodies include any as described herein. In one embodiment, the antibody is aducanumab. In some embodiments, the antibody fusion contains a first heavy chain that is a hole aducanumab heavy chain set forth in SEQ ID NO:131, a linker and a first modified cyclotide; a second heavy chain composed of a knob aducanumab heavy chain set forth in SEQ ID NO: 130, a linker and a second modified cyclotide; and a light chain set forth in SEQ ID NO:132. In some embodiments, the antibody fusion contains a first heavy chain that is a hole trastuzumab heavy chain set forth in SEQ ID NO:136, a linker and a first modified cyclotide; a second heavy chain composed of a knob trastuzumab heavy chain set forth in SEQ ID NO: 135, a linker and a second modified cyclotide; and a light chain set forth in SEQ ID NO:137. In some embodiments, the first and second modified cyclotide are different and are each independently any as described in Section I.B. In some embodiments, modified cyclotides are independently selected from a modified cyclotide that targets TrfR (e.g., set forth in SEQ ID NO:72 or SEQ ID NO:101), targets IgFR (e.g, set forth in SEQ ID NO:81 or SEQ ID NO:82), targets LRP-1 (e.g., SEQ ID NO:85, SEQ ID NO:87 or SEQ ID NO:89), or for targeting RAGE (e.g., SEQ ID NO:83 or SEQ ID NO:84). In some embodiments, one of the first and second modified cyclotide is the modified cyclotide set forth in SEQ ID NO:72 and the other of the first and second modified cyclotide is the cyclotide set forth in SEQ ID NO:82. In some embodiments, one of the first and second modified cyclotide is the modified cyclotide set forth in SEQ ID NO:101 and the other of the first and second modified cyclotide is the cyclotide set forth in SEQ ID NO:82. Any of various combinations of modified cyclotides, such as any described in Section I.B, can be used. In some embodiments, the linker is a GS linker of at least 10 amino acids in length, such as at least 15 amino acids in length. In some embodiments the GS linker is 10 to 25 amino acids. In some embodiments, the GS linker is set forth in SEQ ID NO:104. Also provided herein are antibody fusion conjugates having a sequence that has at least 90%, 91%, 92%, 93%, 94%, 05%, 96%, 97%, 98%, 99% or more sequence identity to any of the foregoing amino acid sequences. In some embodiment, the modified cyclotide binds to the BBB-R recognized by the modified cyclotide to to transport the antibody across the blood brain barrier.

In some embodiments, any of the provided conjugates, such as fusion proteins, bind to a BBB-R, which binding is conferred by the peptide of the modified cyclotide of the conjugate. Methods for determining binding affinity, or relative binding affinity, are known in art, solid-phase ELISA immunoassays, ForteBio Octet, Biacore measurements or flow cytometry. See, for example, Larsen et al., American Journal of Transplantation, vol. 5: 443-453 (2005); Linsley et al., Immunity, Vol 1 (9): 793-801 (1994). In some embodiments, binding affinity can be measured by flow cytometry, such as based on a Mean Fluorescence Intensity (MFI) in a flow binding assay. In some embodiments, the provided conjugate, such as fusion protein, has a binding affinity for a BBB-R as determined by, for example, solid-phase ELISA immunoassays, flow cytometry or surface plasmon resonance (Biacore) assays.

In some embodiments, the conjugate (e.g. fusion protein) has a binding affinity for a BBB-R of greater than 10 nM and less than 1000 nM. In some embodiments, the conjugate has a binding affinity for a BBB-R of greater than 20 nM and less than 800 nM. In some embodiments, the conjugate has a binding affinity for a BBB-R of about 40 nM, 50 nM, 60 nM, 70 nM, 80 nM, 100 nM, 150 nM, 200 nM, 250 nM, 300 nM, 350 nM, 400 nM, 450 nM, 500 nM, 550 nM, 600 nM, 650 nM, 700 nM, 750 nM or any value between any of the foregoing. In some embodiments, the conjugate has a binding affinity for a BBB-R of from about 50 nM to about 500 nM. In some embodiments, the conjugate has a binding affinity for a BBB-R of from about 50 nM to about 250 nM, In some embodiments, the conjugate has a binding affinity for a BBB-R of from about 50 nM to about 100 nM. In some embodiments, the conjugate has a binding affinity for a BBB-R of from about 100 nM to about 500 nM. In some embodiments, the conjugate has a binding affinity for a BBB-R of from about 100 nM to about 250 nM. In some embodiments, the conjugate has a binding affinity for a BBB-R of from about 250 nM to 500 nM. In some embodiments, the binding affinity is about 50 nM to 500 nM.

In embodiments in which the conjugate (e.g. fusion protein) contains a cleavable linker, such as an endosome-specific cleavable linker (e.g. cathspein cleavable linker) the conjugate can have a higher binding affinity for the BBB-R. In some embodiments, the conjugate (e.g. fusion protein) has a binding affinity for a BBB-R of 50 nM or less, such as 1 pM to 10 nM.

In some embodiments, the conjugate containing a modified cyclotide exhibits improved or enhanced delivery of the bioactive or therapeutic agent to the brain compared to an unconjugated form of the bioactive or therapeutic agent that is not conjugated to the modified cyclotide. In some embodiments, the delivery is improved or enhanced by about 1.2-fold, about 1.3-fold, 1.4-fold, 1.5-fold, 1.8-fold, 2.0-fold, 2.5-fold, 3.0-fold, 3.5-fold, 4.0-fold, 4.5-fold, 5.0-fold, 5.5-fold or more. In some embodiments, a conjugate as provided delivers greater than 0.1% of the bioactive or therapeutic agent injected into the bloodstream (e.g., by subcutaneous or intravenous injection) to the brain. In some embodiments, of the dose injected into the blood stream (e.g., by subcutaneous or intravenous injection) a conjugate as provided delivers greater than 0.2%, greater than 0.25%, greater than 0.3%, greater than 0.35%, greater than 0.40% or more into the brain.

### III. NUCLEIC ACIDS AND METHODS OF MAKING

A further embodiment provided herein provides for a nucleic acid sequence that encodes the amino acid sequence of any of the provided binding molecules, such as any of the provided modified cyclotides or fusion proteins containing the same. In particular embodiments, the nucleic acid sequence may be comprised within a vector, suitably an expression vector, optionally a display vector (including phage or cis-display vectors), where the encoding DNA is operably linked to the peptide.

Also provided herein are methods of making the provided binding molecules, such as modified cyclotides or fusion proteins containing the same. In some embodiments, the methods include culturing a host cells under conditions permissive for expression of the fusion protein. In some embodiments, the provided methods further include, following the culturing, isolating the expressed modified cyclotide or fusion protein from the supernatant or from a lysate of the host cell.

In any of the above provided embodiments, the nucleic acids encoding the binding molecule, such as any of the provided modified cyclotides or fusion proteins, provided herein can be introduced into cells using recombinant DNA and cloning techniques. To do so, a recombinant DNA molecule encoding a polypeptide is prepared. Methods of preparing such DNA molecules are well known in the art. In some embodiments, the DNA molecule could be synthesized using chemical synthesis techniques, such as the phosphoramidite method. In some instances, a recombinant or synthetic nucleic acid may be generated through polymerase chain reaction (PCR). In some embodiments, the DNA can be cloned into an appropriate transduction/transfection vector as is known to those of skill in the art. Also provided are expression vectors containing the nucleic acid molecules.

In some embodiments, the expression vectors are capable of expressing the binding molecules, such as modified cyclotides or fusion proteins containing the same, in an appropriate cell under conditions suited to expression of the protein. In some aspects, nucleic acid molecule or an expression vector comprises the DNA molecule that encodes the modified cyclotide or fusion protein operatively linked to appropriate expression control sequences. Methods of effecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

Also provided are vectors that include any of the nucleic acid sequence encoding a binding molecule, such as a modified cyclotide or fusion protein. Vectors suitable for use include those commonly used in genetic engineering technology, such as bacteriophages, plasmids, cosmids, viruses, or retroviruses. Vectors suitable for use may include other expression control elements, such as a transcription starting site, a transcription termination site, a ribosome binding site, a RNA splicing site, a polyadenylation site, a translation termination site, etc. Vectors suitable for use i may further include additional regulatory elements, such as transcription/translation enhancer sequences, and at least a marker gene or reporter gene allowing for the screening of the vectors under suitable conditions. Marker genes suitable for use include, for instance, dihydrofolate reductase gene and G418 or neomycin resistance gene useful in eukaryotic cell cultures, and ampicillin, streptomycin, tetracycline or kanamycin resistance gene useful in E. coli and other bacterial cultures. Vectors suitable for use in this invention may further include a nucleic acid sequence encoding a secretion signal. These sequences are well known to those skilled in the art.

In some embodiments, the expression vector further includes a promoter sequence to control the expression of the modified cyclotide or fusion protein. The term “promoter sequence” as used herein refers to a DNA sequence, which is generally located upstream of a gene present in a DNA polymer, and provides a site for initiation of the transcription of said gene into mRNA. Promoter sequences suitable for use may be derived from viruses, bacteriophages, prokaryotic cells or eukaryotic cells, and may be a constitutive promoter or an inducible promoter.

In some embodiments, the promoter sequence is operably linked to the sequence encoding the modified cyclotide or fusion protein. The term “operatively linked” as used herein means that a first sequence is disposed sufficiently close to a second sequence such that the first sequence can influence the second sequence or regions under the control of the second sequence. For instance, a promoter sequence may be operatively linked to a gene sequence, and is normally located at the 5′-terminus of the gene sequence such that the expression of the gene sequence is under the control of the promoter sequence. In addition, a regulatory sequence may be operatively linked to a promoter sequence so as to enhance the ability of the promoter sequence in promoting transcription. In such case, the regulatory sequence is generally located at the 5′-terminus of the promoter sequence.

Promoter sequences suitable for use include any one of the following: viruses, bacterial cells, yeast cells, fungal cells, algal cells, plant cells, insect cells, animal cells, and human cells. For example, a promoter useful in bacterial cells includes, but is not limited to, tac promoter, T7 promoter, T7 A1 promoter, lac promoter, trp promoter, trc promoter, araBAD promoter, and XPRPL promoter. A promoter useful in plant cells includes, e.g., 35S CaMV promoter, actin promoter, ubiquitin promoter, etc. Regulatory elements suitable for use in mammalian cells include CMV-HSV thymidine kinase promoters, SV40, RSV-promoters, CMV enhancers, or SV40 enhancers.

Depending on the vector and host cell system used, the recombinant gene product (protein) produced may either remain within the recombinant cell, be secreted into the culture medium, be secreted into periplasm, or be retained on the outer surface of a cell membrane. The recombinant gene product (protein) produced by the method can be purified by using a variety of standard protein purification techniques, including, but not limited to, affinity chromatography, ion exchange chromatography, gel filtration, electrophoresis, reverse phase chromatography, chromatofocusing and the like. The recombinant gene product (protein) produced by the method is preferably recovered in “substantially pure” form. As used herein, the term “substantially pure” refers to a purity of a purified protein that allows for the effective use of said purified protein as a commercial product.

In some embodiments, the provided methods for producing a binding molecule, such as a modified cyclotide or fusion protein containing the same, can be performed using any host organism or cell which is capable of expressing heterologous polypeptides, and is capable of being genetically modified. For purposes of clarity, the term “host cell” will be used herein throughout, but it should be understood, that a host organism can be substituted for the host cell, unless unfeasible for technical reasons.

In some embodiments, the host cells can be a variety of eukaryotic cells, such as in yeast cells, or with mammalian cells such as Chinese hamster ovary (CHO) or HEK293 cells. In some embodiments, the host cell is a suspension cell and the polypeptide is engineered or produced in cultured suspension, such as in cultured suspension CHO cells, e.g. CHO-S cells. In some examples, the cell line is a CHO cell line that is deficient in DHFR (DHFR-), such as DG44 and DUXB11. In some embodiments, the cell is deficient in glutamine synthase (GS), e.g. CHO-S cells, CHOK1 SV cells, and CHOZN((R)) GS−/− cells. In some embodiments, the CHO cells, such as suspension CHO cells, may be CHO-S-2H2 cells, CHO-S-clone 14 cells, or ExpiCHO-S cells.

In some embodiments, host cells can also be prokaryotic cells, such as with E. coli. The transformed recombinant host is cultured under polypeptide expressing conditions, and then purified to obtain a soluble protein. Recombinant host cells can be cultured under conventional fermentation conditions so that the desired polypeptides are expressed. Such fermentation conditions are well known in the art. Finally, the polypeptides provided herein can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, and affinity chromatography. Protein refolding steps can be used, as desired, in completing configuration of the mature protein. Finally, high performance liquid chromatography (HPLC) can be employed in the final purification steps.

In some embodiments, polypeptides provided herein can also be made by synthetic methods. Solid phase synthesis is the preferred technique of making individual peptides since it is the most cost-effective method of making small peptides. For example, well known solid phase synthesis techniques include the use of protecting groups, linkers, and solid phase supports, as well as specific protection and deprotection reaction conditions, linker cleavage conditions, use of scavengers, and other aspects of solid phase peptide synthesis. Peptides can then be assembled into the polypeptides as provided herein.

### IV. PHARMACEUTICAL COMPOSITIONS

Also provided herein in some embodiments is a pharmaceutical composition comprising any of the providing molecules of any of the preceding embodiments, including any of the modified cyclotides or conjugates or fusion proteins containing the same, and a pharmaceutically acceptable excipient. In some embodiments, said pharmaceutical composition is used for the treatment of a neurological disease. In some embodiments, said pharmaceutical composition is used for the diagnosis of a neurological disease.

Such compositions typically contain a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The medicaments and pharmaceutical compositions of the invention can take the form of liquids, solutions, suspensions, lotions, gels, tablets, pills, pellets, powders, modified-release formulations (such as slow or sustained-release), suppositories, emulsions, aerosols, sprays, capsules (for example, capsules containing liquids or powders), liposomes, microparticles or any other suitable formulations known in the art. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

To aid dissolution of the therapeutic agent (comprising the cyclotide) into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. Potential nonionic detergents that could be included in the formulation as surfactants include: lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants, when used, could be present in the formulation of the peptide or nucleic acid or derivative either alone or as a mixture in different ratios.

Additives may be included to further enhance cellular uptake of the cyclotide of the invention, such as the fatty acids oleic acid, linoleic acid and linolenic acid.

A pharmaceutical composition of the disclosure is formulated to be compatible with its intended route of administration. Examples of routes of administration include parenteral, e.g., intravenous, intradermal, subcutaneous, intratumoral, oral (e.g., inhalation), transdermal (i.e., topical), transmucosal, and rectal administration. In particular embodiments, the compositions are formulated for parenteral administration. In some embodiments, the compositions are formulated for subcutaneous or intravenous administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a kit, container, pack, or dispenser together with instructions for administration. These pharmaceutical compositions can be included in diagnostic kits with instructions for use.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated. In some embodiments, the pharmaceutical composition may be administered in an amount in the range of about 50 g/kg body weight to about 50 mg/kg body weight per dose. In some embodiments, the pharmaceutical composition may be administered in an amount in the range of about 100 g/kg body weight to about 50 mg/kg body weight per dose. In some embodiments, the pharmaceutical composition may be administered in an amount in the range of about 100 g/kg body weight to about 20 mg/kg body weight per dose. In some embodiments, the pharmaceutical composition may be administered in an amount in the range of about 0.5 mg/kg body weight to about 20 mg/kg body weight per dose.

In some embodiments, the pharmaceutical composition may be administered in an amount in the range of about 10 mg to about 1,000 mg per dose. In some embodiments, the pharmaceutical composition may be administered in an amount in the range of about 20 mg to about 500 mg per dose. In some embodiments, the pharmaceutical composition may be administered in an amount in the range of about 20 mg to about 300 mg per dose. In some embodiments, the pharmaceutical composition may be administered in an amount in the range of about 20 mg to about 200 mg per dose.

The pharmaceutical composition may be administered as needed to subjects. In some embodiments, an effective dose of the pharmaceutical composition is administered to a subject one or more times. In various embodiments, an effective dose of the pharmaceutical composition is administered to the subject once a month, less than once a month, such as, for example, every two months, every three months, or every six months. In other embodiments, an effective dose of the pharmaceutical composition is administered more than once a month, such as, for example, every two weeks, every week, twice per week, three times per week, daily, or multiple times per day. An effective dose of the pharmaceutical composition is administered to the subject at least once. In some embodiments, the effective dose of the pharmaceutical composition may be administered multiple times, including for periods of at least a month, at least six months, or at least a year. In some embodiments, the pharmaceutical composition is administered to a subject as-needed to alleviate one or more symptoms of a condition.

### V. METHODS OF USE AND TREATMENTS

Also provided herein in some embodiments is a method for transporting a biologically active agent across a blood brain barrier of an individual, the method comprising administering the conjugate (e.g., fusion protein) or the pharmaceutical composition of any of the preceding embodiments to a mammal in need thereof.

Also provided herein in some embodiments is a method for treating a subject having a neurological disease comprising administering the conjugate (e.g., fusion protein) or the pharmaceutical composition of any of the preceding embodiments to said subject. In some embodiments, the conjugates (e.g., fusion proteins) or pharmaceutical compositions comprising the same are administered to a subject in an effective amount to effect treatment of the neurological disease. Also provided herein are uses of the conjugates (e.g., fusion proteins) or pharmaceutical compositions containing the same in such methods and treatments, and in the preparation of a medicament in order to carry out such therapeutic methods. In some embodiments, the methods are carried out by administering the conjugates (e.g., fusion protein), or compositions comprising the same, to the subject having, having had, or suspected of having the neurological disease. In some embodiments, the methods thereby treat the neurological disease in the subject. Also provided herein are of use of any of the compositions, such as pharmaceutical compositions provided herein, for the treatment of a neurological disease.

Also provided herein in some embodiments is a method for diagnosing a neurological disease in a patient in need thereof comprising administering the conjugate or the pharmaceutical composition of any of the preceding embodiments to said patient and wherein said conjugate comprises a radiolabel.

In some embodiments, the mammal has a neurological disease. In some embodiments, the neurological disease is selected from the group consisting of Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, and traumatic brain injury. In some embodiments, said neurological disease is selected from the group consisting of Alzheimer's disease, Parkinson's disease, stroke, a brain tumor, a brain metastasis or a traumatic brain injury (TBI). In some embodiments, said neurological disease is a congenital disease that is selected from the group consisting of Austen Disease, Canavan Disease, Gaucher's Disease, Hunter Syndrome, Hurler-Scheie Syndrome, Jansky Bielschowsky Disease, Krabbe Disease, LCAT Deficiency, Lowe Syndrome, Maroteaux-Lamy Syndrome, Morquio Syndrome A, Morquio Syndrome B, Sanfilippo Syndrome A, Sanfilippo Syndrome B, Sanfilippo Syndrome C, Sanfilippo Syndrome D, Spinal Muscular Atrophy, Tay Sachs Disease, and Walker-Warburg Syndrome.

In some embodiments, the neurological disease includes non-neoplastic diseases and disorders, such as cancers/neoplastic diseases and related conditions). In some embodiments, the neurological disease is an intracranial neoplasms.

In some embodiments, the neurological disease or disorder is for treatment of strokes or other brain damaging conditions. In some embodiments, a cyclotide of the invention is conjugated to a therapeutic agent capable of enhancing cognitive ability such as memory.

In some embodiments, a conjugate (e.g. fusion protein) containing a modified cyclotide linked to a glucocerebrosidase is administered to a subject for treating a neurodegenerative disease. In some embodiments, the neurological disease is Gaucher's disease. In some embodiments, the neurological disease is Parkison's Disease. The glucocerebrosidase and conjugates containing the same can be any as descried in Section II. Glucocerebrosidase is a lysosomal luminal membrane-associated hydrolase, where it interacts with glucolipids. Inherited autosomal, recessive mutations of the GBA gene encoding glucocerebrosidase (GCase) lead to deficiency in activity and the clinical manifestation of Gaucher disease (GD) (Beutler and Grabowski, 1994). Mutations in GBA are also frequently found in Parkison disease patients. When dysfunctional, the accumulation of substrate induces an altered inflammatory response in tissue macrophages (Gaucher cells) and other related cells (Komhaber et al, 2008; Schetz & Shankar, 2004; Smith et al, 2017). Gaucher cells are the most prominent pathological hallmark together with mononuclear phagocytes and neuronal cells in the brain, which are involved in the pathology of GD (Beutler & Grabowski, 2001).

In some embodiments, the neurological disease is Gaucher's disease. Symptoms of GD include anemia, enlargement of liver and spleen, bone lesions and in severe cases neurologic manifestations, widely classified into three clinical subtypes (Beutler et al, 1994; Komhaber et al, 2008). The most common form is classified into type I Gaucher disease with nearly no neuronopathic implications, currently treatable with the use of enzyme replacement therapy (ERT). Type II and III are more severe types of GD with type II having acute neuronopathic phenotype, indicating symptoms at near birth, progressing until death during early infancy. Type III causes chronic severe neuropathic symptoms, including learning disabilities, cardiac abnormalities and myolonic epilepsy (Beutler et al, 1994; Sidransky et al, 2007; Sidransky & Lopez, 2012). In some embodiments, the provided methods are for treating type I Gaucher disease. In some embodiments, the provided methods are for treating type II Gaucher disease. In some embodiments, the provided methods are for treating type III Gaucher disease.

In some embodiments, the subject to be treated has a mutation in the natural glucocerebrosidase gene (also called GCase or GBA gene), which is associated with the neurological disease, such as GD, in the subject. Approximately, 300 mutations have been identified and directly linked to the progression of GD, resulting in a wide pathological spectrum of this disorder (Alfonso et al, 2007; Grabowski & Horowitz, 1997). Inherited missense and nonsense mutations in the GBA gene can lead to misfolding, mistrafficking and destabilization of the glucocerebrosidase enzyme. Two of the most frequent mutations found in the literature are the mutations N370S and L444P, accounting for 50% of all known GBA mutations. In some embodiments of the provided methods, the subject carries the GCase mutation N370S and/or L444P. Patients carrying the mutated GCase variant N370S are usually diagnosed with type I GD, possessing a wide range of symptoms. N370S is responsible for conserving and stabilizing proper conformation of the active binding pocket (Lieberman et al, 2007). L444P reveals a more severe type categorized in type II or III when carrying this GBA gene mutation, possibly disrupting the hydrophobic structure, altering the domain II function (Lieberman et al, 2007).

In some embodiments, a conjugate (e.g. fusion protein) containing a modified cyclotide linked to an anti-HER2 antibody is administered to a subject for treating a neurological disease or disorder. The anti-HER2 antibody and conjugates containing the same can be any as described in Section II. In some embodiments, the anti-HER2 antibody is trastuzumab. In some embodiments, the conjugate is used for treating a metastatic brain cancer. In some embodiments, the conjugates are used for treating Alzheimer's Disease.

In some embodiments, a conjugate (e.g. fusion protein) containing a modified cyclotide linked to an anti-TNF-alpha antibody is administered to a subject for treating a neurological disease or disorder. The anti-TNF-alpha antibody and conjugates containing the same can be any as described in Section II. In some embodiments, the anti-TNF-alpha antibody is adalimumab. In some embodiments, the neurological disease or disorder is a stroke, traumatic brain injury or Alzheimer's Disease.

In some embodiments, a conjugate (e.g. fusion protein) containing a modified cyclotide linked to an anti-amyloid beta antibody is administered to a subject for treating a neurological disease or disorder. The anti-amyloid beta antibody and conjugates containing the same can be any as described in Section II. In some embodiments, the anti-amyloid beta antibody is aducanumab. In some embodiments, the neurological disease or disorder is Alzheimer's Disease.

In some embodiments, a conjugate (e.g. fusion protein) containing a modified cyclotide linked to an nerve growth factor (NGF) is administered to a subject for treating a neurological disease or disorder. The NGF and conjugates containing the same can be any as described in Section II. In some embodiments, the neurological disease or disorder is Parkinson's Disease, stroke or Alzheimer's Disease.

In some embodiments, a conjugate (e.g. fusion protein) containing a modified cyclotide linked to a granulocyte colony-stimulating factor (GCSF) is administered to a subject for treating a neurological disease or disorder. The GCSF and conjugates containing the same can be any as described in Section II. In some embodiments, the neurological disease or disorder is stroke or a traumatic brain injury.

In some embodiments, a conjugate (e.g. fusion protein) containing a modified cyclotide linked to a interleukin 10 (IL-10) is administered to a subject for treating a neurological disease or disorder. The IL-10 and conjugates containing the same can be any as described in Section II. In some embodiments, the neurological disease or disorder is Alzheimer's Disease.

In some embodiments, a conjugate (e.g. fusion protein) containing a modified cyclotide linked to a brain-derived neurotrophic factor (BDNF) is administered to a subject for treating a neurological disease or disorder. The BDNF and conjugates containing the same can be any as described in Section II. In some embodiments, the neurological disease or disorder is ALS or depression.

In some embodiments, a conjugate (e.g. fusion protein) containing a modified cyclotide linked to a activity-dependent neuroprotective protein (ADNP) is administered to a subject for treating a neurological disease or disorder. The ADNP and conjugates containing the same can be any as described in Section II. In some embodiments, the neurological disease or disorder is an autism-like disorder.

In some embodiments, a conjugate (e.g. fusion protein) containing a modified cyclotide linked to a heparin N Sulfatase is administered to a subject for treating a neurological disease or disorder. The heparin N Sulfatase and conjugates containing the same can be any as described in Section II. In some embodiments, the neurological disease or disorder is Sanfilippo A.

In some embodiments, a conjugate (e.g. fusion protein) containing a modified cyclotide linked to a galactocerebrosidase is administered to a subject for treating a neurological disease or disorder. The galactocerebrosidase and conjugates containing the same can be any as described in Section II. In some embodiments, the neurological disease or disorder is Krabbe's disease.

In some embodiments, a conjugate (e.g. fusion protein) containing a modified cyclotide linked to an alpha galactosidase is administered to a subject for treating a neurological disease or disorder. The alpha galactosidase and conjugates containing the same can be any as described in Section II. In some embodiments, the neurological disease or disorder is Fabry's disease.

In some embodiments, a conjugate (e.g. fusion protein) containing a modified cyclotide linked to a sphingomyelinase is administered to a subject for treating a neurological disease or disorder. The sphingomyelinase and conjugates containing the same can be any as described in Section II. In some embodiments, the neurological disease or disorder is Niemann Pick.

In some embodiments, a conjugate (e.g. fusion protein) containing a modified cyclotide linked to a cerliponase alpha is administered to a subject for treating a neurological disease or disorder. The cerliponase alpha and conjugates containing the same can be any as described in Section II. In some embodiments, the neurological disease or disorder is Jansky Bielschowsky.

In some embodiments, a conjugate (e.g. fusion protein) containing a modified cyclotide linked to a alpha glucosidase is administered to a subject for treating a neurological disease or disorder. The alpha glucosidase and conjugates containing the same can be any as described in Section II. In some embodiments, the neurological disease or disorder is Pompe's.

In some embodiments, a conjugate (e.g. fusion protein) containing a modified cyclotide linked to a tripeptidyl peptidase I is administered to a subject for treating a neurological disease or disorder. The tripeptidyl peptidase I and conjugates containing the same can be any as described in Section II. In some embodiments, the neurological disease or disorder is Jansky Bieschowsky or Batten.

In some embodiments, a conjugate (e.g. fusion protein) containing a modified cyclotide linked to a galactosamine 6 sulfatase is administered to a subject for treating a neurological disease or disorder. The galactosamine 6 sulfatase and conjugates containing the same can be any as described in Section II. In some embodiments, the neurological disease or disorder is Marquio syndrome.

In some embodiments, the methods and uses include administering a provided conjugate (e.g., fusion protein) or a pharmaceutical composition comprising the same into a subject (e.g., a human). In some embodiments, the administration to the subject by a parenteral administration. In some embodiments, the administration is intramuscularly, subcutaneously, intravenously, topically, orally or by inhalation. In some embodiments, the administration is intramuscular. In some embodiments, the administration is subcutaneous.

In some embodiments, a provided conjugate (e.g., fusion protein) is administered to the subject in an effective or therapeutically effective amount. In some embodiments, the effective or therapeutically effective dose is a dose for treating a neurological disorder. In some embodiments, the effective or therapeutically effective dose is an amount sufficient to alleviate one or more signs and/or symptoms of the neurological disorder in the treated subject, whether by inducing the regression or elimination of such signs and/or symptoms or by inhibiting the progression of such signs and/or symptoms. The dose amount may vary depending upon the age and the size of a subject to be administered, target disease, conditions, route of administration, and the like. In an embodiment, an effective or therapeutically effective dose of a provided conjugate (e.g., fusion protein) for treating a neurological disorder, e.g., in an adult human subject, is about 0.001 mg/kg to about 200 mg/kg, such as 0.01 mg/kg to 200 mg/kg or 0.1 mg/kg to 200 mg/kg. Depending on the severity of the infection, the frequency and the duration of the treatment can be adjusted.

In specific embodiments of the present invention, the cyclotide-conjugated therapeutic molecules of the present invention are utilized as separately administered compositions or in conjunction with other therapeutic agents. These additional agents can include various immunotherapeutic drugs, such as cyclosporine, methotrexate, adriamycin or cisplatinum, and immunotoxins, or chemotherapeutic drugs such as tamoxifen, paclitaxel, oxaliplatin, vincristine and fluorouracil. Pharmaceutical compositions can include combinations of various cytotoxic or other agents in conjunction with the polypeptides of the present invention.

### VI. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

A number of definitions are provided that will assist in the understanding of the invention. All references cited herein are incorporated by reference in their entirety. Unless otherwise defined, all technical and scientific terms used herein have the same meaning as commonly understood by one of ordinary skill in the art to which this invention belongs.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known in this technical field. Reference to “about” a value or parameter herein includes (and describes) embodiments that are directed to that value or parameter per se. For example, description referring to “about X” includes description of “X”.

The term “conjugate” is used in its broadest sense to encompass all methods of attachment or joining that are known in the art. Such conjugates include fusion proteins, those produced by chemical conjugates and those produced by any other methods. The term “conjugated” is used interchangeably with terms such as “linked”, “bound”, “associated”, “fused” or “attached”. A wide range of covalent and non-covalent forms of conjugation are known to the person of skill in the art, and fall within the scope of the invention. For example, disulphide bonds, chemical linkages and peptide chains are all forms of covalent linkages. Where a non-covalent means of conjugation is preferred, the means of attachment may be, for example, a biotin-(strept)avidin link or the like. Antibody (or antibody fragment)-antigen interactions may also be suitably employed to conjugate a cyclotide of the invention to another moiety, such as a polypeptide, peptide, non-polypeptide moiety, small molecule, or biological drug.

“Binding affinity” generally refers to the strength of the sum total of noncovalent interactions between a single binding site of a molecule (e.g., an antibody) and its binding partner (e.g., an antigen). Unless indicated otherwise, “binding affinity” refers to intrinsic binding affinity which reflects a 1:1 interaction between members of a binding pair (e.g., antibody and antigen). The affinity of a molecule X for its partner Y can generally be represented by the dissociation constant. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure.

“Percent (%) amino acid sequence identity” with respect to a peptide or polypeptide sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MegAlign (DNASTAR) software. Those skilled in the art can determine appropriate parameters for measuring alignment, including any algorithms needed to achieve maximal alignment over the full length of the sequences being compared.

“Amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

“Conservatively modified variants” applies to both amino acid and nucleic acid sequences. “Amino acid variants” refers to amino acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product, but not with respect to actual probe sequences. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” including where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles disclosed herein. Typically conservative substitutions include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “polypeptide” is a polymer of amino acid residues joined by peptide bonds, whether produced naturally or in vitro by synthetic means. In some embodiments, a polymer of amino acids of 2 to 50 amino amino acids typically referred to as “peptides”. The term “polypeptide” as used herein denotes the product of a naturally occurring polypeptide, precursor form or proprotein. The term also appies to amino acid polymers in which one or more amino acid residue is an artificial chemical mimetic of a corresponding naturally occurring amino acid, as well as to naturally occurring amino acid polymers and non-naturally occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like. The term “protein” is used herein to refer to a large polypeptide molecule as well as a macromolecule comprising one or more polypeptide chains.

The term “peptide” as used herein refers to a plurality of amino acids joined together, such as in a linear or circular chain. The term oligopeptide is typically used to describe peptides having between 2 and about 50 or more amino acids. Peptides larger than about 50 are often referred to as polypeptides or proteins.

A “non-polypeptide moiety” as used herein, refers to an entity that does not contain an polypeptide sequence or three-dimensional fold. The person of skill in the art understands and can determine whether a polypeptide molecule is an polyprotein or peptide sequence, for example, by way of sequence homology or structure prediction or determination. Such non-polypeptide moieties include nucleic acids and other polymers, peptides, proteins, peptide nucleic acids (PNAs), antibodies, antibody fragments, and small molecules, amongst others. Suitably, a non-polypeptide moiety is a biological molecule (e.g. comprising a polynucleotide or peptide), and advantageously is a therapeutic or targeting molecule.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (VH) regions capable of specifically binding, and single chain variable fragments (scFv).

An “antibody fragment” comprises a portion of an intact antibody, the antigen binding and/or the variable region of the intact antibody. Antibody fragments, include, but are not limited to, Fab fragments, Fab′ fragments, F(ab′)₂ fragments, Fv fragments, disulfide-linked Fvs (dsFv), Fd fragments, Fd′ fragments; single-chain antibody molecules, including single-chain Fvs (scFv) or single-chain Fabs (scFab); antigen-binding fragments of any of the above and multispecific antibodies from antibody fragments.

A “Fab fragment” is an antibody fragment that results from digestion of a full-length immunoglobulin with papain, or a fragment having the same structure that is produced synthetically, e.g., by recombinant methods. A Fab fragment contains a light chain (containing a V_{L} and C_{L}) and another chain containing a variable domain of a heavy chain (V_{H}) and one constant region domain of the heavy chain (C_{H}1).

An “scFv fragment” refers to an antibody fragment that contains a variable light chain (V_{L}) and variable heavy chain (V_{H}), covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

The term “operably linked”, when applied to DNA sequences, for example in an expression vector or construct indicates that the sequences are arranged so that they function cooperatively in order to achieve their intended purposes, i.e. a promoter sequence allows for initiation of transcription that proceeds through a linked coding sequence as far as the termination sequence.

The term “nucleic acid sequence” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acid sequences may include DNA and RNA, and may be manufactured synthetically in vitro or isolated from natural sources. Sizes of nucleic acid sequences, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

The term “vector” is used to denote a DNA molecule that is either linear or circular, into which another nucleic acid (typically DNA) sequence fragment of appropriate size can be integrated. Such DNA fragment(s) can include additional segments that provide for transcription of a gene encoded by the DNA sequence fragment. The additional segments can include and are not limited to: promoters, transcription terminators, enhancers, internal ribosome entry sites, untranslated regions, polyadenylation signals, selectable markers, origins of replication and such like. A variety of suitable promoters for prokaryotic (e.g. the β-lactamase and lactose promoter systems, alkaline phosphatase, the tryptophan (trp) promoter system, lac, tac, T3, T7 promoters for E. coli) and eukaryotic (e.g. simian virus 40 early or late promoter, Rous sarcoma virus long terminal repeat promoter, cytomegalovirus promoter, adenovirus late promoter, EG-la promoter) hosts are available. Expression vectors are often derived from plasmids, cosmids, viral vectors and yeast artificial chromosomes; vectors are often recombinant molecules containing DNA sequences from several sources.

Specific embodiments provide for an expression vector that encodes a modified polypeptide or fragment/domain thereof that comprise a molecule of the invention. Accordingly, the DNA encoding a relevant peptide of the invention can be inserted into a suitable expression vector (e.g. pGEM®, Promega Corp., USA), where it is operably linked to appropriate expression sequences, and transformed into a suitable host cell for protein expression according to conventional techniques (Sambrook J. et al., Molecular Cloning: a Laboratory Manual, Cold Spring Harbor Press, Cold Spring Harbor, NY). Suitable host cells are those that can be grown in culture and are amenable to transformation with exogenous DNA, including bacteria, fungal cells and cells of higher eukaryotic origin, preferably mammalian cells, typically human cells and cell lines. To aid in purifying the peptides of the invention, the polypeptide (and corresponding nucleic acid) of the invention may include a purification sequence, such as a His-tag. In addition, or alternatively, the modified polypeptides may, for example, be grown in fusion with another protein and purified as insoluble inclusion bodies from bacterial cells. This is particularly convenient when the modified polypeptide to be synthesised may be toxic to the host cell in which it is to be expressed. Alternatively, modified polypeptides may be synthesised in vitro using a suitable in vitro (transcription and) translation system (e.g. the E. coli S30 extract system: Promega corp., USA).

In one embodiment of the present invention the vector is suitable as a polypeptide library display vector, enabling the polypeptide gene product of the cyclotide encoding gene to remain associated with the vector following transcription.

As used herein, the term “polypeptide library display” refers to a system in which a collection of polypeptides or peptides, that may form part or all of a library, are made available for selection based upon a specified characteristic. The specified characteristic may be a physical, chemical or functional characteristic. Suitable display systems utilise a cellular expression system, for instance an expression of a library of nucleic acids in appropriately transformed, infected, transfected or transduced cells and display of the encoded polypeptides on the surface of the cells. Alternative cellular expression systems may include emulsion compartmentalization and display. Optional display systems link the coding function of a nucleic acid and physical, chemical and/or functional characteristics of a polypeptide or peptide encoded by the nucleic acid. When such a display system is employed, polypeptides or peptides that have a desired physical, chemical and/or functional characteristic can be selected and the nucleic acid encoding the selected polypeptide is readily isolated. Several display systems that link the coding functionality of a nucleic acid with the associated polypeptide product are known in the art, for example, bacteriophage display (phage display), ribosome display, emulsion compartmentalization and display, yeast display, puromycin display, bacterial display, display on plasmid, covalent display, CIS display and the like. (See, e.g., EP 0436597 (Dyax), U.S. Pat. No. 6,172,197 (McCafferty et al.), U.S. Pat. No. 6,489,103 (Griffiths et al.).

The term “library” refers to a mixture of heterogeneous polypeptides or nucleic acids. The library is composed of a plurality of members, each of which has a substantially unique polypeptide or nucleic acid sequence. Sequence differences between library members are responsible for the diversity present in the library. In the present invention the library may take the form of a simple mixture of polypeptides or nucleic acids, or may be in the form of organisms or cells, for example bacteria, viruses, animal or plant cells and the like, transformed with a library of nucleic acids. Usually, each individual organism (such as a phage) or cell contains only one or a very limited number of library members. Advantageously, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member—i.e. the polypeptide gene product. Thus, the population of host organisms has the potential to encode a widely diverse number of polypeptides. An embodiment of the present invention provides for a library of polypeptides that are based around modified versions of cyclobody polypeptides, in which the diversity or variance between library members is located in the polypeptide sequences of one or more of the loop or variable regions of one or more functional modules within the protein.

By “derived from” it is meant that the cyclotide concerned includes one or more amino acid modifications, such as insertions, deletions or mutations, in comparison to the primary amino acid sequence of the cyclotide on which it is based. Hence, a derivative of a cyclotide also is referred to as a “modified cyclotide.” For instance, a derivative of a cyclotide polypeptide may contain one or more (e.g. 1, 2, 3, 4, 5 or more) amino acid mutations, substitutions or deletions to the primary sequence of a selected modified polypeptide. Accordingly, the invention encompasses the results of maturation experiments conducted on a cyclotide polypeptide to improve or alter one or more characteristics of the initial or unmodified cyclotide. By way of example, one or more amino acid residues of a selected cyclotide polypeptide sequence may be randomly or specifically mutated (or substituted) using procedures known in the art (e.g. by modifying the encoding DNA or RNA sequence). The resultant library or population of derivatised polypeptides may be selected—by any known method in the art—according to predetermined requirements: such as improved specificity against particular target receptors (e.g. receptors involved in transcytosis); improved transcytosis across the blood-brain barrier; or improved drug properties (e.g. solubility, bioavailability, immunogenicity etc.).

The term “isolated” with reference to binding molecules, such as peptides or antibodies or other binding molecules, or polynucleotides and vectors encoding same, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated binding molecule may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

The term “effective amount” or “therapeutically effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, “disease or disorder” refers to a pathological condition in an organism resulting from cause or condition including, but not limited to, infections, acquired conditions, genetic conditions, and characterized by identifiable symptoms.

As used herein, the terms “treat,” “treating,” or “treatment” refer to ameliorating a disease or disorder, e.g., slowing or arresting or reducing the development of the disease or disorder, e.g., a root cause of the disorder or at least one of the clinical symptoms thereof.

As used herein, the term “subject” refers to an animal, including a mammal (e.g., rat, mouse, cat, dog, cow, pig, sheep, horse, goat, rabbit), such as a human being. Typically the subject is a human subject. The term subject and patient can be used interchangeably.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

### VII. EXEMPLARY EMBODIMENTS

Among the provided embodiments are:
1. A modified cyclotide comprising:
i) a peptide that binds to a blood-brain barrier trancytosis receptor (BBB-R) selected from the group consisting of transferrin receptor (TrfR), insulin-like growth factor type 1 receptor (IGFR), Erb-B2 Receptor Tyrosine Kinase 3 (ErbB3), leptin receptor (ObR), low-density lipoprotein receptor-related protein 1 (LRP-1), and receptor for advanced glycation-end products (RAGE), wherein said peptide has an amino acid sequence of 2 to 50 amino acid residues; and
ii) a cyclotide scaffold comprising the peptide of i),
   wherein the modified cyclotide comprises the structure (I):

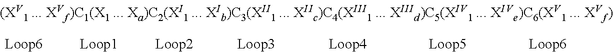

wherein C₁ to C₆ are cysteine residues; wherein each of C₁ and C₄, C₂ and C₅, and C₃ and C₆ are connected by a disulfide bond to form a cysteine knot; wherein each X represents an amino acid residue in a loop, wherein said amino acid residues are the same or different; wherein d is about 1-2; wherein at least one loop from loops 1, 2, 3, 5 or 6 has an amino acid sequence comprising the sequence of said peptide of clause i), wherein any loop comprising said sequence of said peptide of clause i) comprises 2 to about 30 amino acids, and wherein for any of loops 1, 2, 3, 5, or 6 that do not contain said sequence of said peptide of clause i), a, b, c, e, and f, are the same or different, and are each any number from 3-10, and b, c, e, and f are each any number from 1 to 20.
2. The modified cyclotide of embodiment 1, wherein the BBB-R is human.
3. The modified cyclotide of embodiment 1 or embodiment 2, wherein the cyclotide scaffold is selected from a plant cyclotide.
4. The modified cyclotide of any of embodiments 1-3, wherein the cyclotide scaffold is of the Momordicae species.
5. The modified cyclotide of embodiment 4, wherein the cyclotide scaffold is a Momordica cochinchinensis trypsin inhibitor.
6. The modified cyclotide of embodiment 5, wherein the Momordica cochinchinensis trypsin inhibitor is MCoTI-I set forth in SEQ ID NO: 1, MCoTI-II set forth in SEQ ID NO: 2 or MCoTI-III set forth in SEQ ID NO: 3.
7. The modified cyclotide of any of embodiments 1-6, wherein the sequence of said peptide replaces or substitutes one or more amino acids of one of the one or more loops of the cyclotide scaffold.
8. A modified cyclotide comprising a peptide that binds to a blood-brain barrier trancytosis receptor (BBB-R), wherein: the peptide is inserted into or replaces one or more amino acids of at least one loop of the cyclotide scaffold set forth in SEQ ID NO:1, SEQ ID NO:2 or SEQ ID NO: 3, and wherein the peptide is about 2 to 50 amino acid residues; and the BBB-R is selected from the group consisting of transferrin receptor (TrfR), insulin-like growth factor type 1 receptor (IGFR), Erb-B2 Receptor Tyrosine Kinase 3 (ErbB3), leptin receptor (ObR), low-density lipoprotein receptor-related protein 1 (LRP-1), and
9. The modified cyclotide of any of embodiments 1-8, wherein the cyclotide scaffold is set forth in SEQ ID NO:2.
10. The modified cyclotide of any of embodiments 1-9, wherein the at least one loop is loop 1, loop 5 or loop 6, or is a combination thereof.
11. The modified cyclotide of any of embodiments 1-10, wherein the at least one loop is loop 1.
12. The modified cyclotide of any of embodiments 1-11, wherein the peptide is inserted into and replaces amino acids in only one loop of the cyclotide scaffold.
13. The modified cyclotide of embodiment 12, wherein the only loop is loop 1.
14. The modified cyclotide of any of embodiments 11-13, wherein the cyclotide scaffold is set forth in SEQ ID NO:2 and the peptide replaces loop 1 amino acids between cysteine 4 and cysteine 11 of SEQ ID NO:2.
15. The modified cyclotide of any of embodiments 1-14, wherein all amino acids of the at least one loop are replaced by the peptide.
16. A modified cyclotide comprising a peptide inserted into loop 1 to replace all amino acids between cysteine 4 and cysteine 11 of SEQ ID NO:2, wherein the peptide is 2 to 50 amino acid residues and binds to a blood-brain barrier trancytosis receptor (BBB-R) selected from the group consisting of transferrin receptor (TrfR), insulin-like growth factor type 1 receptor (IGFR), Erb-B2 Receptor Tyrosine Kinase 3 (ErbB3), leptin receptor (ObR), low-density lipoprotein receptor-related protein 1 (LRP-1).
17. The modified cyclotide of any of embodiments 1-16, wherein the peptide is 2 to 40 amino acids, 2 to 30 amino acids, 2 to 25 amino acids, 2 to 20 amino acids, 2 to 15 amino acids, 2 to 10 amino acids, 2 to 5 amino acids, 5 to 50 amino acids, 5 to 40 amino acids, 5 to 30 amino acids, 5 to 25 amino acids, 5 to 20 amino acids, 5 to 15 amino acids, 5 to 10 amino acids, 10 to 50 amino acids, 10 to 40 amino acids, 10 to 30 amino acids, 10 to 25 amino acids, 10 to 15 amino acids, 15 to 50 amino acids, 15 to 40 amino acids, 15 to 30 amino acids, 15 to 25 amino acids, 15 to 20 amino acids, 20 to 50 amino acids, 20 to 40 amino acids, 20 to 30 amino acids, 20 to 25 amino acids, 25 to 50 amino acids, 25 to 40 amino acids, 25 to 30 amino acids, 30 to 50 amino acids, 30 to 40 amino acids, or 40 to 50 amino acids.
18. The modified cyclotide of any of embodiments 1-16, wherein the peptide is 2 to 30 amino acids, such as 2 to 24 amino acids, 2 to 18 amino acids, 2 to 12 amino acids, 2 to 6 amino acids, 6 to 30 amino acids, 6 to 24 amino acids, 6 to 18 amino acids, 6 to 12 amino acids, 12 to 30 amino acids, 12 to 24 amino acids, 12 to 18 amino acids, 18 to 30 amino acids, 18 to 24 amino acids or 24 to 30 amino acids.
19. The modified cyclotide of any of embodiments 1-16, wherein the peptide is 14 to 20 amino acids.
20. The modified cyclotide of any of embodiments 1-18, wherein the peptide is 10 amino acids.
21. The modified cyclotide of any of embodiments 1-18, wherein the peptide is 12 amino acids.
22. The modified cyclotide of any of embodiments 1-21, wherein the BBB-R is expressed on brain endothelial cells.
23. The modified cyclotide of any of embodiments 1-22, wherein the peptide has blood-brain barrier translocation activity.
24. The modified cyclotide of any of embodiments 1-23, wherein the BBB-R is the transferrin receptor.
25. The modified cyclotide of any of embodiments 1-24, wherein the peptide comprises the sequence set forth in any one of SEQ ID NOS: 26-34 and 49-54, optionally wherein the peptide is set forth in any one of SEQ ID NOS: 26-34 and 49-54.
26. The modified cyclotide of any of embodiments 1-24, wherein the peptide has the consensus motif set forth as CxxxxxHxxSWGxC (SEQ ID NOL:177).
27. The modified cyclotide of any of embodiments 1-26, wherein the modified cyclotide comprises the sequence set forth in any one of SEQ ID NOS: 72-80 and 95-100, optionally wherein the modified cyclotide is set forth in any one of SEQ ID NOS: 72-80 and 95-100.
28. The modified cyclotide of any of embodiments 1-24 and 26, wherein the peptide comprises the sequence forth in SEQ ID NO:26, optionally wherein the peptide is set forth in SEQ ID NO:26.
29. The modified cyclotide of any of embodiments 1-28, wherein the modified cyclotide comprises the sequence set forth in SEQ ID NO:72, optionally wherein the modified cyclotide is set forth in SEQ ID NO:72.
30. The modified cyclotide of any of embodiments 1-29, wherein the peptide comprises a sequence with 1, 2, 3 or 4 amino acid substitution(s) compared to the sequence set forth in any one of SEQ ID NOS: 26-34 and 49-54, optionally wherein the amino acid substitution(s) is to a histidine or an alanine.
31. The modified cyclotide of any of embodiments 1-24 and 30, wherein the peptide comprises the sequence set forth in any one of SEQ ID NOS: 55 and 117-128.
32. The modified cyclotide of any of embodiments 1-24 and 30, wherein the peptide is set forth in any one of SEQ ID NOS: 55 and 117-128.
33. The modified cyclotide of any of embodiments 1-24 and 30-32, wherein the peptide comprises the sequence set forth in SEQ ID NO:55, optionally wherein the peptide is set forth in SEQ ID NO:55.
34. The modified cyclotide of any of embodiments 1-23, wherein the BBB-R is the leptin receptor.
35. The modified cyclotide of any of embodiments 1-23 and 34, wherein the peptide comprises the sequence set forth in any one of SEQ ID NOS: 10-23.
36. The modified cyclotide of any of embodiments 1-23, 34 and 35, wherein the peptide is set forth in any one of SEQ ID NOS: 10-23.
37. The modified cyclotide of any of embodiments 1-23 and 34-36, wherein the modified cyclotide comprises the sequence set forth in any one of SEQ ID NOS: 56-69, optionally wherein the modified cyclotide is set forth in any one of SEQ ID NOS: 56-69.
38. The modified cyclotide of any of embodiments 1-23 and 34, wherein the peptide comprises a sequence with 1, 2, 3 or 4 amino acid substitution(s) compared to the sequence set forth in any one of SEQ ID NOS: 10-23, optionally wherein the amino acid substitution(s) is to a histidine or an alanine.
39. The modified cyclotide of any of embodiments 1-23, wherein the BBB-R is ErbB3.
40. The modified cyclotide of any of embodiments 1-23 and 39, wherein the peptide comprises the sequence set forth in SEQ ID NO:24 or 25.
41. The modified cyclotide of any of embodiments 1-23, 39 and 40, wherein the peptide is set forth in SEQ ID NO: 24 or 25.
42. The modified cyclotide of any of embodiments 1-23 and 39-41, wherein the modified cyclotide comprises the sequence set forth SEQ ID NO: 70 or 71, optionally wherein the modified cyclotide is set forth in SEQ ID NO: 70 or 71.
43. The modified cyclotide of any of embodiments 1-23 and 39, wherein the peptide comprises a sequence with 1, 2, 3 or 4 amino acid substitution(s) compared to the sequence set forth in SEQ ID NO: 24 or 25, optionally wherein the amino acid substitution(s) is to a histidine or an alanine.
44. The modified cyclotide of any of embodiments 1-23, wherein the BBB-R is insulin-like growth factor type 1 receptor (IGFR).
45. The modified cyclotide of any of embodiments 1-23 and 44, wherein the peptide comprises the sequence set forth in SEQ ID NO:35 or 36.
46. The modified cyclotide of any of embodiments 1-23, 44 and 45, wherein the peptide is set forth in SEQ ID NO: 35 or 36.
47. The modified cyclotide of any of embodiments 1-23 and 44-46, wherein the peptide comprises the sequence set forth in SEQ ID NO:36, optionally wherein the peptide is set forth in SEQ ID NO:36.
48. The modified cyclotide of any of embodiments 1-23 and 44-47, wherein the modified cyclotide comprises the sequence set forth SEQ ID NO: 81 or 82, optionally wherein the modified cyclotide is set forth in SEQ ID NO: 81 or 82.
49. The modified cyclotide of any of embodiments 1-23 and 44-48, wherein the modified cyclotide comprises the sequence set forth in SEQ ID NO:82, optionally wherein the modified cyclotide is set forth in SEQ ID NO:82.
50. The modified cyclotide of any of embodiments 1-23 and 44, wherein the peptide comprises a sequence with 1, 2, 3 or 4 amino acid substitution(s) compared to the sequence set forth in SEQ ID NO: 35 or 36, optionally wherein the amino acid substitution(s) is to a histidine or an alanine.
51. The modified cyclotide of any of embodiments 1-23, wherein the BBB-R is RAGE.
52. The modified cyclotide of any of embodiments 1-23 and 51, wherein the peptide comprises the sequence set forth in SEQ ID NO:37 or 38.
53. The modified cyclotide of any of embodiments 1-23, 51 and 52, wherein the peptide is set forth in SEQ ID NO: 37 or 38.
54. The modified cyclotide of any of embodiments 1-23 and 51-53, wherein the modified cyclotide comprises the sequence set forth SEQ ID NO: 83 or 84, optionally wherein the modified cyclotide is set forth in SEQ ID NO: 83 or 84.
55. The modified cyclotide of any of embodiments 1-23 and 51, wherein the peptide comprises a sequence with 1, 2, 3 or 4 amino acid substitution(s) compared to the sequence set forth in SEQ ID NO: 37 or 38, optionally wherein the amino acid substitution(s) is to a histidine or an alanine.
56. The modified cyclotide of any of embodiments 1-23, wherein the BBB-R is the 1 lipoprotein receptor-related protein 1 (LRP-1).
57. The modified cyclotide of any of embodiments 1-23 and 56, wherein the peptide comprises the sequence set forth in any one of SEQ ID NOS: 39-48.
58. The modified cyclotide of any of embodiments 1-23, 56 and 57, wherein the peptide is set forth in any one of SEQ ID NOS: 39-48.
59. The modified cyclotide of any of embodiments 1-23 and 56-58, wherein the modified cyclotide comprises the sequence set forth in any one of SEQ ID NOS: 85-94, optionally wherein the modified cyclotide is set forth in any one of SEQ ID NOS: 85-94.
60. The modified cyclotide of any of embodiments 1-23 and 56, wherein the peptide comprises a sequence with 1, 2, 3 or 4 amino acid substitution(s) compared to the sequence set forth in any one of SEQ ID NOS: 39-48, optionally wherein the amino acid substitution(s) is to a histidine or an alanine.
61. A peptide comprising the amino acid sequence set forth in any of SEQ ID NOs: 10-55 or 117-128, wherein the peptide is 6-50 amino acids in length and binds to a receptor involved in blood-brain barrier transcytosis (BBB-R).
62. The peptide of embodiment 61, wherein the peptide has blood-brain barrier translocation activity.
63. The peptide of embodiment 61 or embodiment 62, wherein the peptide is 2 to 40 amino acids, 2 to 30 amino acids, 2 to 25 amino acids, 2 to 20 amino acids, 2 to 15 amino acids, 2 to 10 amino acids, 2 to 5 amino acids, 5 to 50 amino acids, 5 to 40 amino acids, 5 to 30 amino acids, 5 to 25 amino acids, 5 to 20 amino acids, 5 to 15 amino acids, 5 to 10 amino acids, 10 to 50 amino acids, 10 to 40 amino acids, 10 to 30 amino acids, 10 to 25 amino acids, 10 to 15 amino acids, 15 to 50 amino acids, 15 to 40 amino acids, 15 to 30 amino acids, 15 to 25 amino acids, 15 to 20 amino acids, 20 to 50 amino acids, 20 to 40 amino acids, 20 to 30 amino acids, 20 to 25 amino acids, 25 to 50 amino acids, 25 to 40 amino acids, 25 to 30 amino acids, 30 to 50 amino acids, 30 to 40 amino acids, or 40 to 50 amino acids.
64. The peptide of any of embodiments 61-63, wherein the peptide is 2 to 30 amino acids, such as 2 to 24 amino acids, 2 to 18 amino acids, 2 to 12 amino acids, 2 to 6 amino acids, 6 to 30 amino acids, 6 to 24 amino acids, 6 to 18 amino acids, 6 to 12 amino acids, 12 to 30 amino acids, 12 to 24 amino acids, 12 to 18 amino acids, 18 to 30 amino acids, 18 to 24 amino acids or 24 to 30 amino acids.
65. The peptide of any of embodiments 61-64, wherein the peptide is 14 to 20 amino acids.
66. The peptide of any of embodiments 61-64, wherein the peptide is 10 amino acids.
67. The peptide of any of embodiments 61-64, wherein the peptide is 12 amino acids.
68. A peptide consisting of the sequence set forth in any one of SEQ ID NOs: SEQ ID NOs: 10-55 or 117-128.
69. The peptide of embodiment 68, wherein the peptide has blood-brain barrier translocation activity.
70. The peptide of embodiment 68 or embodiment 69, wherein the peptide binds to a receptor involved in blood-brain barrier transcytosis.
71. A peptide set forth by the sequence of any of SEQ ID NOS: 26-34 and 49-55 and 117-128, wherein the peptide binds the transferrin receptor.
72. The peptide of any of embodiments 61-71, wherein the peptide is set forth in SEQ ID NO:26.
73. The peptide of any of embodiments 61-71, wherein the peptide is set forth in SEQ ID NO: 55.
74. A peptide set forth by the sequence of any of SEQ ID NOS: 10-23, wherein the peptide binds the leptin receptor.
75. A peptide set forth by the sequence of any of SEQ ID NOS: 24 or 25, wherein the peptide binds ErbR3.
76. A peptide set forth by the sequence of any of SEQ ID NOS: 35 or 36, wherein the peptide binds IGFR.
77. The peptide of embodiment 76, wherein the peptide is set forth in SEQ ID NO:36.
78. A peptide set forth by the sequence of SEQ ID NO: 37 or 38, wherein the peptide binds the RAGE.
79. A peptide set forth by the sequence of any of SEQ ID NOS: 39-48, wherein the peptide binds the lipoprotein receptor-related protein 1 (LRP-1).
80. The peptide of any of embodiments 61-79, wherein the peptide is synthetic.
81. The peptide of any of embodiments 61-79, wherein the peptide is isolated.
82. A binding molecule comprising a binding scaffold and the peptide of any of embodiments 61-81.
83. The binding molecule of embodiment 82, wherein the binding scaffold is a cyclotide.
84. The binding molecule of embodiment 83 wherein the peptide is inserted into or replaces one or more amino acids of a loop of a cyclotide backbone, optionally wherein the at least one loop is loop 1.
85. The binding molecule of embodiment 83 or embodiment 84, wherein the binding scaffold is set forth in any one of SEQ ID NOS: 1-3.
86. The binding molecule of any of embodiments 83-85, wherein the binding scaffold is set forth in SEQ ID NO:2.
87. A nucleic acid molecule encoding the modified cyclotide of any of embodiments 1-60 or the binding molecule of any of embodiments 82-86.
88. A vector comprising the nucleic acid molecule of embodiment 87.
89. The vector of embodiment 88 that is an expression vector.
90. A host cell comprising the nucleic acid molecule of embodiment 87 or the vector of embodiment 87 or embodiment 89.
91. A method of producing a modified cyclotide or binding molecule, the method comprising introducing the nucleic acid of embodiment 87 or the vector of embodiment 88 or embodiment 89 into a host cell and culturing the host cell under conditions to express the protein in the cell, optionally purifying the protein from the cell.
92. A purified binding molecule or modified cyclotide produced by the method of embodiment 91.
93. A conjugate comprising: a modified cyclotide of any of embodiments 1-60 or binding molecule of any of embodiments 82-86, and a biologically active agent.
94. The conjugate of embodiment 93, wherein the biologically active agent is a small molecule, a peptide or a protein.
95. The conjugate of embodiment 93 or embodiment 94, wherein the biologically active agent is a diagnostic agent or a therapeutic agent.
96. The conjugate of any of embodiments 93-95 that is a fusion protein comprising the modified cyclotide operably linked to a biologically active agent that is a protein or peptide.
97. A fusion protein, comprising a modified cyclotide of any of embodiments 1-60 or binding molecule of any of embodiments 82-86, and a biologically active protein agent.
98. The conjugate of any of embodiments 93-96 or the fusion protein of embodiment 97, wherein the biologically active agent is an antibody.
99. The conjugate or fusion protein of embodiment 98, wherein the antibody is trastuzumab, adalimumab or aducanumab.
100. The conjugate of any of embodiments 93-96 or the fusion protein of embodiment 97, wherein the biologically active agent is a growth factor or a hormone.
101. The conjugate or fusion protein of embodiment 100 wherein the biologically active agent is a growth factor and the growth factor is nerve growth factor (NGF) or Granulocyte colony-stimulating factor (GCSF).
102. The conjugate of any of embodiments 93-96 or the fusion protein of embodiment 97, wherein the biologically active agent is an enzyme.
103. The conjugate or fusion protein of embodiment 102, wherein the enzyme is a ceramide degrading enzyme, a lipase, a hydrolase type enzyme or a sulfatase.
104. The conjugate or fusion protein of embodiment 102 or 103, wherein the enzyme is a ceramide degrading enzyme and the ceramide degrading enzyme is glucocerebrosidase, galactocerebrosidase or alpha galactosidase.
105. The conjugate or fusion protein of embodiment 102 or embodiment 103, wherein the enzyme is a lipase or a hydrolase type enzyme and the enzyme is sphingomyelinase, cerliponase or alpha glucosidase.
106. The conjugate or fusion protein of any of embodiments 93-105 comprising a single modified cyclotide.
107. The conjugate or fusion protein of any of embodiments 93-105 comprising 2, 3, or 4 modified cyclotides.
108. The conjugate or fusion protein of embodiment 107, wherein each modified cyclotide is the same.
109. The conjugate or fusion protein of embodiment 107, wherein each modified cyclotide is different.
110. The conjugate or fusion protein of any of embodiments 93-107 and 108, that is monovalent for binding a BBB-R.
111. The conjugate or fusion protein of any of embodiments 93-105, 107 and 109, comprising at least two different modified cyclotides that bind to different BBB-R.
112. The conjugate or fusion protein of embodiment 111 that is bispecific for binding two different BBB-R.
113. The conjugate or fusion protein of any of embodiments 93-112, wherein the modified cyclotide is linked to the biologically active agent via a linker.
114. The conjugate or fusion protein of embodiment 113, wherein the linker is a flexible peptide linker, optionally comprising the sequence GGGGS (SEQ ID NO:148), (GGGGS)₂ (SEQ ID NO:154) or (GGGGS)₃ (SEQ ID NO:154).
115. The conjugate or fusion protein of embodiment 113 or embodiment 114, wherein the linker is set forth in SEQ ID NO: 104.
116. The conjugate or fusion protein of embodiment 113, wherein the linker is a cleavable linker comprising an endosome-specific protease cleavage site.
117. The conjugate or fusion protein of embodiment 116, wherein the endosome-specific protease cleavage site is a cathepsin cleavage site.
118. The conjugate or fusion protein of embodiment 117, wherein the cathepsin cleavage site is a cathepsin B cleavage site.
119. The conjugate or fusion protein of any of embodiments 116-118, wherein the linker comprises the sequence set forth in SEQ ID NO:133.
120. A pharmaceutical composition comprising the conjugate or fusion protein of any of embodiments 93-119, and a pharmaceutical carrier.
121. A method for transporting a biologically active agent across a blood brain barrier of an individual, the method comprising administering the conjugate or fusion protein of any of embodiments 93-119 or the pharmaceutical composition of embodiment 120 to a mammal in need thereof.
122. The method of embodiment 121, wherein the mammal has a neurological disease.
123. The method of embodiment 121, wherein said neurological disease is selected from the group consisting of Alzheimer's disease, Parkinson's disease, stroke, a brain tumor and a brain metastasis.
124. The method of embodiment 121 or embodiment 123, wherein said neurological disease is a congenital disease that is selected from the group consisting of Austen Disease, Canavan Disease, Gaucher's Disease, Hunter Syndrome, Hurler-Scheie Syndrome, Jansky Bielschowsky Disease, Krabbe Disease, LCAT Deficiency, Lowe Syndrome, Maroteaux-Lamy Syndrome, Morquio Syndrome A, Morquio Syndrome B, Sanfilippo Syndrome A, Sanfilippo Syndrome B, Sanfilippo Syndrome C, Sanfilippo Syndrome D, Spinal Muscular Atrophy, Tay Sachs Disease, and Walker-Warburg Syndrome.
125. A method for treating a patient having a neurological disease comprising administering the conjugate of any of embodiments 46-52 or the pharmaceutical composition of embodiment 53 to said patient.
126. A method for diagnosing a neurological disease in a patient in need thereof comprising administering the conjugate or fusion protein of any of embodiments 93-119 or the pharmaceutical composition of embodiment 53 to said patient and wherein said conjugate comprises a radiolabel.
127. The pharmaceutical composition of embodiment 120 for use in the treatment of a neurological disease.
128. The pharmaceutical composition of embodiment 120 for use in the diagnosis of a neurological disease.

### VIII. EXAMPLES

The following examples are included for illustrative purposes only and are not intended to limit the scope of the invention.

### Example 1: Cyclotide (CYC) Library Construction

Cyclotide libraries were created by insertion of random peptides into the backbone cyclotide MCOTII scaffold set forth in SEQ ID NO:2. The libraries were based on the MCOTII native DNA sequence (SEQ ID NO:4) as a cyclotide scaffold and were built containing mutant sequences between the first two cysteine residues of SEQ ID NO:2, (termed “Loop1”), in which the libraries differed in the number of randomized peptides. To create the libraries, oligonucleotides were designed to insert the mutant sequences into Loop 1 to generate a library with a loop of 10 amino acids (“Loop1for10”; SEQ ID NO:5) or 12 amino acids (“Loop1for12”, SEQ ID NO: 6) when expressed. A further library was similarly generated to encode 14-20 randomized amino acids in Loop1. All randomised amino acid positions were encoded in the library to represent an equal mixture of codons for 19 of the naturally occurring amino acids excluding cysteine.

PCR products were cloned as NcoI-NotI digested fragments into similarly digested pSP1 phagemid pIII fusion vector derived from the pHEN1 pIII vector (Hoogenboom et al., 1991, Nucleic Acids Res., 19: 4133-4137).

### (i) PCR Amplification of Cyclotide Loop Libraries

For the primary PCR amplifications 10× 50 μl amplifications were set up for the Loop libraries using the appropriate oligonucleotide primers (“Loop1for10” or “Loop1for12” SEQ ID NOs: 5 or 6 and pIIIsegrev SEQ ID NO:7). Each 50 μl reaction mixture contained 10 ng pCyclo1, 25 pmol of the appropriate forward and reverse primers, 0.1 mM dNTPs, 2.5 units Taq DNA polymerase, and 1×NEB PCR reaction buffer (20 mM Tris-HCl pH 8.8, 10 mM (NH₄)₂SO₄, 10 mM KCl, 2 mM MgSO₄, 0.1% Triton X-100; NEB Ltd, Cambridge, UK). Reactions were performed for 30 PCR cycles of 94° C., 20s; 60° C., 40s; 72° C., 30s, followed by 5 minutes at 72° C. Reaction products were purified using two Wizard PCR clean-up columns per repertoire (Promega Ltd, Southampton, UK), and eluted into 50 μl water per column.

### (ii) Pull-Through Re-Amplification of Selected DNA

To prepare the final cyclotide library DNA products, 40×50 μl amplifications were set up for each cyclotide loop library, using oligonucleotide primers “Loop1PTrev” and “Loop5PTrev” (SEQ ID NOs: 8 and 9). Each 50 μl reaction mixture contained approximately 25 ng primary Cyclotide Loop library, 25 pmol of the appropriate forward and reverse primers, 0.1 mM dNTPs, 2.5 units Taq DNA polymerase, and 1×NEB PCR reaction buffer (20 mM Tris-HCl pH 8.8, 10 mM (NH₄)₂SO₄, 10 mM KCl, 2 mM MgSO₄, 0.1% Triton X-100; NEB Ltd, Cambridge, UK). Reactions were performed for 25 cycles of 94° C., 20s; 60° C., 40s; 72° C., 30s, followed by 5 minutes at 72° C. Reaction products were purified using four Wizard PCR clean-up columns per library (Promega Ltd, Southampton, UK), and eluted into 100 μl water per column.

(iii) Cloning into Vector pSP1

Each of the libraries, and 250 μg pSP1 vector DNA were digested with enzymes NcoI and NotI (100 units each enzyme) for 5 hours at 37° C. (NEB, Cambridge, UK), and purified using one Wizard PCR clean-up column per library, and four Wizard PCR clean-up columns for the digested vector DNA (Promega Ltd, Southampton, UK). Each DNA sample was then eluted into 100 μl water. Half of each digested library DNA was ligated overnight at 16° C. in 400 μl with 50 μg of NcoI-NotI cut pSP1 vector and 4000U of T4 DNA ligase (NEB Ltd, Southampton, UK). After incubation the ligations were adjusted to 200 μl with nuclease free water, and DNA precipitated with 1 μl 20 mg/ml glycogen, 100 μl 7.5M ammonium acetate and 900 μl ice-cold (−20° C.) absolute ethanol, vortex mixed and spun at 13,000 RPM for 20 minutes in a microfuge to pellet DNA. The pellets were washed with 500 μl ice-cold 70% ethanol by centrifugation at 13,000 RPM for 2 minutes, then vacuum dried and re-suspended in 100 μl DEPC-treated water. 1 μl aliquots of each library were electroporated into 80 μl E. coli (TG1). Bacterial cells were grown in 1 ml SOC medium per cuvette for 1 hour at 37° C., and plated onto 2× TY agar plates supplemented with 2% glucose and 100 μg/ml ampicillin. 10⁻⁴, 10⁻⁵ and 10⁻⁶ dilutions of the electroporated bacteria were also plated to assess library size. Colonies were allowed to grow overnight at 30° C. Combined library size was approximately 2×10¹⁰ clones with >95% with in-frame inserts.

### (iv) Phage Amplification

Separate phage stocks were prepared for each cyclotide library. The bacteria were then scraped off the plates into 50 ml 2× TY broth supplemented with 20% glycerol, 2% glucose and 100 μg/ml ampicillin. 1 ml of bacterial medium was added to a 50 ml 2× TY culture broth supplemented with 1% glucose and 100 μg/ml ampicillin and infected with 10¹¹ kanamycin resistance units (kru) M13K07 helper phage at 37° C. for 30 minutes without shaking, then for 30 minutes with shaking at 200 RPM. Infected bacteria were transferred to 200 ml 2× TY broth supplemented with 25 μg/ml kanamycin, 100 μg/ml ampicillin, and 20 μM IPTG, then incubated overnight at 30° C., shaking at 200 RPM. Bacteria were pelleted at 4000 RPM for 20 minutes in 50 ml Falcon tubes, and 40 ml 2.5 M NaCl/20% PEG 6000 was added to 400 ml of particle supernatant, mixed vigorously and incubated on ice for 1 hour to precipitate phage particles. Particles were pelleted at 11000 RPM for 30 minutes in 250 ml Oakridge tubes at 4° C. in a Sorvall RC5B centrifuge, then resuspended in 40 ml water and 8 ml 2.5M NaCl/20% PEG 6000 added to reprecipitate particles, then incubated on ice for 20 minutes. Particles were again pelleted at 11000 RPM for 30 minutes in 50 ml Oakridge tubes at 4° C. in a Sorvall RC5B centrifuge, then resuspended in 5 ml PBS buffer, after removing all traces of PEG/NaCl with a pipette. Bacterial debris was removed via centrifugation for 5 minutes at 13500 RPM in a microcentrifuge. The supernatant was filtered through a 0.45 μm polysulfone syringe filter, adjusted to 20% glycerol and stored at −70° C.

### Example 2: Library Selections Against Blood-Brain Barrier Transport Receptors

Cyclotide phage libraries described in Example 1 were screened for receptor binding peptides able to bind a receptor capable of inducing receptor-mediating transcytosis (RMT) across the blood-brain barrier (BBB), including human HER3/ErbB3 (Hu ErbB3), human and mouse transferrin receptor (Hu TrfR or Mu TrfR), human leptin receptor (Hu ObR), human insulin-like growth factor type 1 receptor (Hu IGFR), human low-density lipoprotein receptor-related protein 1 (Hu LRP-1), and human receptor for advanced glycation-end products (Hu RAGE).

Selections were carried out using standard phage display selection methods on solid phase coated with recombinant proteins composed of the extracellular domain of each of the human (Hu) or mice (Mu) receptors (recombinant Fc fusion proteins obtained from R&D Systems, or His-tagged receptors from Sino Biologicals).

For TfR cyclotide selection, a round of selection was also carried out in the presence of human holo-transferrin, such that selections were carried out against human TfR when human holo-transferrin is bound to the receptor. In this selection, CYC40 (SEQ ID NO:95) was the most predominant peptide hit in this selection.

Following phage ELISA to identify receptor binding cyclotides, plasmid DNA was then prepared and sequences of individual cyclotide clones was determined via sequencing. Binding specificity was confirmed by ELISA.

Table E1 sets forth sequences of selected cyclotides and the receptors they bind.

TABLE El
Cyclotide and receptor binding peptide target and sequences

| | | Receptor binding peptides | | Cyclotides | |
|---|---|---|---|---|---|
| | | | SEQ ID | | SEQ ID |
| Name | Target | Peptide Sequence | NO | Full Cyclotide Sequence | NO |
| CYC1 | Hu ObR | PWPKWTYMAQKY | 10 | GGVCPWPKWTYMAQKYCRRDSDC | 56 |
| | | | | PGACICRGNGYCGSGSD | |
| CYC2 | Hu ObR | QSFFWWHSWFGV | 11 | GGVCQSFFWWHSWFGVCRRDSDC | 57 |
| | | | | PGACICRGNGYCGSGSD | |
| CYC3 | Hu ObR | HSQFWPWEWYQF | 12 | GGVCHSQFWPWEWYQFCRRDSDC | 58 |
| | | | | PGACICRGNGYCGSGSD | |
| CYC4 | Hu ObR | WRWTPKIWVLEEM | 13 | GGVCWRWTPKIWVLEEMSSCSDCP | 59 |
| | | SS | | GACICRGNGYCGSGSD | |
| CYC5 | Hu ObR | WQGMMLFFVGEK | 14 | GGVCWQGMMLFFVGEKCRRDSDC | 60 |
| | | | | PGACICRGNGYCGSGSD | |
| CYC6 | Hu ObR | GWFYWYHKWFGP | 15 | GGVCGWFYWYHKWFGPCRRDSDC | 61 |
| | | | | PGACICRGNGYCGSGSD | |
| CYC7 | Hu ObR | YDWGWFWPFEQY | 16 | GGVCYDWGWFWPFEQYCRRDSDC | 62 |
| | | | | PGACICRGNGYCGSGSD | |
| CYC8 | Hu ObR | SDRWDWMFATWL | 17 | GGVCSDRWDWMFATWLCRRDSDC | 63 |
| | | | | PGACICRGNGYCGSGSD | |
| CYC9 | Hu ObR | VQPWYSWFMEQG | 18 | GGVCVQPWYSWFMEQGCRRDSDC | 64 |
| | | | | PGACICRGNGYCGSGSD | |
| CYC10 | Hu ObR | PTQTWWFWLPAQ | 19 | GGVCPTQTWWFWLPAQCRRDSDC | 65 |
| | | | | PGACICRGNGYCGSGSD | |
| CYC11 | Hu ObR | YVDWPWTLTLWY | 20 | GGVCYVDWPWTLTLWYCRRDSDC | 66 |
| | | | | PGACICRGNGYCGSGSD | |
| CYC12 | Hu ObR | EWSDWFPYEFLF | 21 | GGVCEWSDWFPYEFLFCRRDSDCP | 67 |
| | | | | GACICRGNGYCGSGSD | |
| CYC13 | Hu ObR | PTAMWFDWFTVY | 22 | GGVCPTAMWFDWFTVYCRRDSDC | 68 |
| | | | | PGACICRGNGYCGSGSD | |
| CYC14 | Hu ObR | FLWFWYGFSRTL | 23 | GGVCFLWFWYGFSRTLCRRDSDCP | 69 |
| | | | | GACICRGNGYCGSGSD | |
| CYC15 | Hu | RHSYSQIPLW | 24 | GGVCRHSYSQIPLWCRRDSDCPGA | 70 |
| | ErbB3 | | | CICRGNGYCGSGSD | |
| CYC16 | Hu | WWDKWVFPISRQ | 25 | GGVCWWDKWVFPISRQCRRDSDC | 71 |
| | ErbB3 | | | PGACICRGNGYCGSGSD | |
| CYC17 | Hu TrfR | LAHKWHLQSWGT | 26 | GGVCLAHKWHLQSWGTCRRDSDC | 72 |
| | | | | PGACICRGNGYCGSGSD | |
| CYC18 | Hu TrfR | DPRWLAGWGS | 27 | GGVCDPRWLAGWGSCRRDSDCPE | 73 |
| | | | | TCICRGNGYCASGYD | |
| CYC19 | Mu TrfR | TVWPFVWWGGVP | 28 | GGVCTVWPFVWWGGVPCRRDSDC | 74 |
| | | | | PGACICRGNGYCGSGSD | |
| CYC20 | Mu TrfR | AYVHDWYHHVMY | 29 | GGVCAYVHDWYHHVMYCRRDSD | 75 |
| | | | | CPGACICRGNGYCGSGSD | |
| CYC21 | Mu TrfR | MHNSASHNIQMQ | 30 | GGVCMHNSASHNIQMQCRRDSDCP | 76 |
| | | | | GACICRGNGYCGSGSD | |
| CYC22 | Mu TrfR | QTVFPMWPWYQL | 31 | GGVCQTVFPMWPWYQLCRRDSDC | 77 |
| | | | | PGACICRGNGYCGSGSD | |
| CYC23 | Mu TrfR | HNLFVIESILFW | 32 | GGVCHNLFVIESILFWCRRDSDCPG | 78 |
| | | | | ACICRGNGYCGSGSD | |
| CYC24 | Hu TrfR | KALWYMGWGT | 33 | GGVCKALWYMGWGTCRRDSDCPG | 79 |
| | | | | ACICRGNGYCGSGSD | |
| CYC25 | Hu TrfR | KQNWLPGWGT | 34 | GGVCKQNWLPGWGTCRRDSDCPG | 80 |
| | | | | ACICRGNGYCGSGSD | |
| CYC26 | Hu IGFR | FDPFYAMLQQLV | 35 | GGVCFDPFYAMLQQL VCRRDSDCP | 81 |
| | | | | GACICRGNGYCGSGSD | |
| CYC27 | Hu IGFR | QGFYSWFAVQTM | 36 | GGVCQGFYSWFAVQTMCRRDSDC | 82 |
| | | | | PGACICRGNGYCGSGSD | |
| CYC28 | Hu | HVGYWWHIWMGA | 37 | GGVCHVGYWWHIWMGACRRDSD | 83 |
| | RAGE | | | CPGACICRGNGYCGSGSD | |
| CYC29 | Hu | VSNFWFMHWFGV | 38 | GGVCVSNFWFMHWFGVCRRDSDC | 84 |
| | RAGE | | | PGACICRGNGYCGSGSD | |
| CYC30 | Hu LRP- | WLTLGIANKGIDVY | 39 | GGVCWLTLGIANKGIDVYDVKCRR | 85 |
| | 1 | DVK | | DSDCPGACICRGNGYCGSGSD | |
| CYC31 | Hu LRP- | NPLKLKYWREWTY | 40 | GGVCNPLKLKYWREWTYGGGCRR | 86 |
| | 1 | GGG | | DSDCPGACICRGNGYCGSGSD | |
| CYC32 | Hu LRP- | YTQNRYKWLYAQT | 41 | GGVCYTQNRYKWLYAQTLNCRRD | 87 |
| | 1 | LN | | SDCPGACICRGNGYCGSGSD | |
| CYC33 | Hu LRP- | IEDRKHHRNLHQTV | 42 | GGVCIEDRKHHRNLHQTVQCRRDS | 88 |
| | 1 | Q | | DCPGACICRGNGYCGSGSD | |
| CYC34 | Hu LRP- | WYETKTKLYVGVV | 43 | GGVCWYETKTKLYVGVVKCRRDS | 89 |
| | 1 | K | | DCPGACICRGNGYCGSGSD | |
| CYC35 | Hu LRP- | LALTKPYWQWAM | 44 | GGVCLALTKPYWQWAMCRRDSDC | 90 |
| | 1 | | | PGACICRGNGYCGSGSD | |
| CYC36 | Hu LRP- | WHHEWITWWFWH | 45 | GGVCWHHEWITWWFWHCRRDSD | 91 |
| | 1 | | | CPGACICRGNGYCGSGSD | |
| CYC37 | Hu LRP- | DQSPYKLKHYGW | 46 | GGVCDQSPYKLKHYGWCRRDSDC | 92 |
| | 1 | | | PGACICRGNGYCGSGSD | |
| CYC38 | Hu LRP- | PWYKMWFYETHR | 47 | GGVCPWYKMWFYETHRCRRDSDC | 93 |
| | 1 | | | PGACICRGNGYCGSGSD | |
| CYC39 | Hu LRP- | TPWKSQPLFWFQ | 48 | GGVCTPWKSQPLFWFQCRRDSDCP | 94 |
| | 1 | | | GACICRGNGYCGSGSD | |
| CYC40 | Hu TrfR | EYFRWHLDSWGE | 49 | GGVCEYFRWHLDSWGECRRDSDC | 95 |
| | | | | PGACICRGNGYCGSGSD | |
| CYC41 | Hu TrfR | LVSQWHIESWGQ | 50 | GGVCL VSQWHIESWGQCRRDSDCP | 96 |
| | | | | GACICRGNGYCGSGSD | |
| CYC42 | Hu TrfR | QEERWHMHSWGP | 51 | GGVCQEERWHMHSWGPCRRDSDC | 97 |
| | | | | PGACICRGNGYCGSGSD | |
| CYC43 | Hu TrfR | RYQEWHMESWGS | 52 | GGVCRYQEWHMESWGSCRRDSDC | 98 |
| | | | | PGACICRGNGYCGSGSD | |
| CYC44 | Hu TrfR | LYEAWHLPSWGQ | 53 | GGVCLYEA WHLPSWGQCRRDSDC | 99 |
| | | | | PGACICRGNGYCGSGSD | |
| CYC45 | Hu TrfR | QMVSEHWVARVW | 54 | GGVCQMVSEHWVARVWCRRDSD | 100 |
| | | | | CPGACICRGNGYCGSGSD | |

### Example 3: Generation and Assessment of Adalimumab Antibody-Cyclotide Fusions

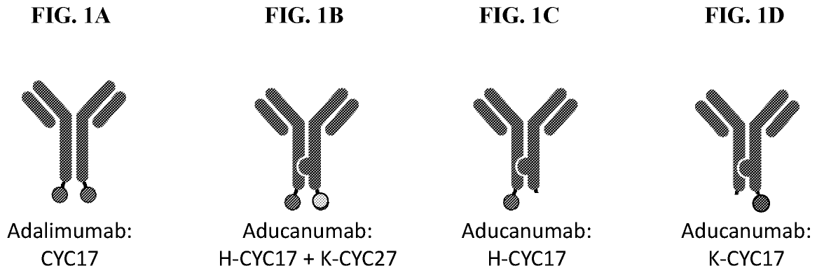

Select cyclotide peptides described in Table E1 were fused with the antibody adalimumab. The cyclotides were linked to the C-terminal end of the polynucleotide encoding the heavy chain (SEQ ID NO:102) or light chain (SEQ ID NO:103) of adalimumab via a Gly-Ser linker (e.g. SEQ ID NO: 104). An exemplary depiction of an adalimumab-cyclotide fusion protein is shown in FIG. 1A.

Genetic constructs for expression of antibody-cyclotide fusions were prepared by cloning the cyclotide gene fused to the 3′ end of a nucleotide sequence coding for the antibody heavy chain gene (with 5′ TL2 secretory signal peptide sequence) into a mammalian expression vector. The adalimumab light chain gene (with 5′ secretory signal peptide sequence) also was cloned into a mammalian expression vector. These two vector constructs were used to co-transfect HEK293F cells, which were grown for 4 to 6 days at 37° C., 8% CO2, 130 rpm shaking. After removal of cells and filtration of the supernatant, the resulting adalimumab-cyclotide fusion was purified by Protein A affinity chromatography and concentrated. Two exemplary different adalimumab-cyclotide fusion proteins showed no signs of enhanced aggregation when compared with the non-cyclotide fused version of the antibody as determined by western blot (data not shown).

### Example 4: Internalization of Transferrin Receptor (TfR)-Targeted Cyclotide-Antibody Fusion

The exemplary cyclotide CYC17 (also called HT2; set forth in SEQ ID NO:72), which binds the human transferrin receptor, was fused to adalimumab. The adalimumab-CYC17 cyclotide fusion was compared to adalimumab for internalization via the transferrin receptor.

### Cloning, Expression, Purification of Adalimumab and Adalimumab-HT2 Cyclotide Fusion

Genetic constructs for expression of the adalimumab heavy chain (SEQ ID NO: 102) and light chain (SEQ ID NO:103) were prepared by cloning the adalimumab heavy chain gene (with 5′ IL2 secretory signal peptide sequence) and adalimumab light chain gene (with 5′ secretory signal peptide sequence), respectively, into mammalian expression vectors.

The CYC17 cyclotide set forth in SEQ ID NO:72 was linked to the C-terminal end of the heavy chain (SEQ ID NO:102) of adalimumab via a Gly-Ser linker (e.g., SEQ ID NO: 104). A genetic construct for expression of the adalimumab-CYC17 cyclotide fusion protein was prepared by cloning the cyclotide gene fused to the 3′ end of a nucleotide sequence coding for the antibody heavy chain gene (with 5′ IL2 secretory signal peptide sequence) into a mammalian expression vector.

The expression constructs for the adalimumab heavy chain-CYC17 cyclotide fusion protein (or adalimumab heavy chain alone) and the adalimumab light chain (amino acid sequences set forth in SEQ ID NO:103) were used to co-transfect HEK293F cells, which were grown for 4 to 6 days at 37° C., 8% CO₂, 130 rpm shaking. After removal of cells and filtration of the supernatant, the resulting adalimumab-CYC17 cyclotide fusion protein was purified by Protein A affinity chromatography and concentrated using a 50 KDa molecular weight cutoff centrifugal filtration unit (Amicon).

### Fluorophore Labeling of Adalimumab and Adalimumab-HT2 Cyclotide Fusion

Purified adalimumab and adalimumab-CYC17 cyclotide fusion proteins were fluorescently labeled by incubation with a 5- to 10-fold molar excess of Alexafluor 488 N-hydroxysuccinamide (NHS) ester for one hour at room temperature, pH ˜8.3 in the dark. Free dye was then removed by passage through one to two desalting columns.

### Preparation of Human Cells Expressing Human or Mouse Transferrin Receptor

HEK293F cells were first serially passaged two to three times, then transfected with expression constructs encoding human or mouse transferrin receptor (hTfR, mTfR, respectively), and grown for 2-3 days at 37° C., 8% CO₂, 130 rpm shaking in preparation for the assay.

### Assaying Internalisation of Labeled Adalimumab and Adalimumab-CYC17 Cyclotide Fusion by Transferrin Receptor-Expressing Human Cells.

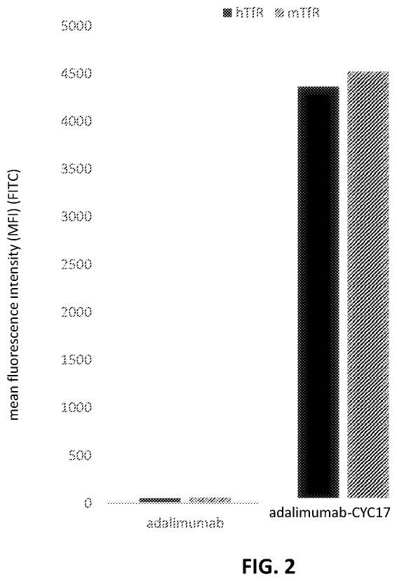

After ˜64 h of growth, 1×10⁵-5×10⁵ transfected cells (and untransfected and mock transfected controls) were transferred to 96-well V-bottom plates, pelleted, supernatant discarded, resuspended in 40 μL fresh media containing 50 nM of labeled adalimumab or adalimumab-CYC17 cyclotide fusion, and grown for 4 h at 37° C., 8% CO₂, 500 rpm shaking. Cells were then pelleted, supernatant discarded, and resuspended in 40 μL fresh media containing 250 nM anti-488 quenching antibody (or media lacking 488 quenching antibody as a control), incubated on ice for 5 min, pelleted, supernatant discarded, washed with 200 μL cold FACS buffer, pelleted, supernatant discarded, and resuspended in 200 μL cold FACS buffer. Each sample was then subjected to FACS analysis using the FITC fluorescence detection channel, using Gate 1 (forward scatter linear vs. side scatter linear) to select live cells with normal morphology, using Gate 2 (side scatter area vs. side scatter linear) to select single cells, and measuring logarithmic FITC signal for 20,000-50,000 cells per sample. Subsequent data analysis was carried out using the FlowJo software package. The mean FITC fluorescence intensity of each sample was monitored as an indication of the extent to which the labeled adalimumab or adalimumab-CYC17 cyclotide fusion was internalized. Results shown in FIG. 2 demonstrate that adalimumab-CYC17 cyclotide fusion, but not adalimumab, was internalized by both the mouse TfR (mTfR) and human TfR (hTfR).

### Example 5: Affinity Modulation of a Transferrin Receptor Binding Cyclotide

Selected TfR-binding cyclotides identified in Table E1 were further assessed for their affinity to TfR and mutant cyclotides were generated with reduced affinity.

Genetic constructs for expression of histidine and alanine scanning mutants of adalimumab-CYC17 cyclotide fusions were prepared. Mutants of CYC17 (SEQ ID NO:72) were prepared by PCR-based assembly of overlapping oligonucleotide primers encoding each CYC17 mutant. Each mutant CYC17 cyclotide gene was fused to the 3′ end of a nucleotide sequence encoding the adalimumab heavy chain gene (with 5′ secretory signal peptide sequence) via a sequence encoding a flexible linker into a mammalian expression vector. The vector construct encoding each adalimumab heavy chain-mutant CYC17 cyclotide fusion and the adalimumab light chain were co-transfected into HEK293F cells, which were grown for 4 to 6 days at 37° C., 8% CO₂, 130 rpm shaking. After removal of cells and filtration of the supernatant, the resulting adalimumab-cyclotide fusion was purified by Protein A affinity chromatography and concentrated.

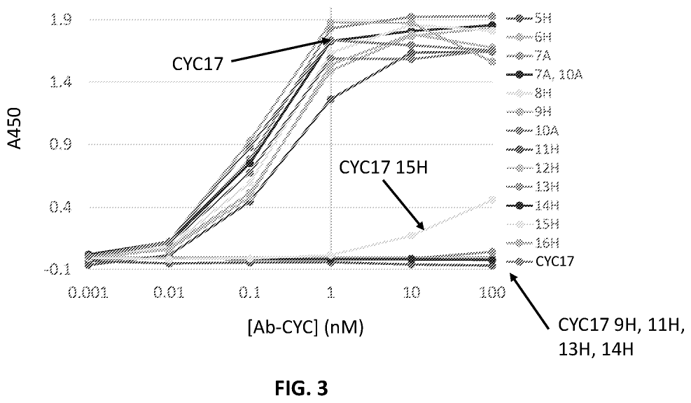

Exemplary CYC17 mutants are set forth in Table E2. Binding changes to human transferrin receptor for the exemplary CYC17 mutants were assessed by ELISA. Briefly, His-tagged human Transferrin receptor (TrFR) was coated at 1 μg/mL on a microwell plate overnight at 4° C., blocked for 1 hour at room temperature with 200 milk powder/PBS and purified-antibody-cyclotide fusions at various concentrations from 0.001 nM to 100 nM were contacted with the coated transferrin receptor, and binding of the antibody cyclotide fusion to the coated human transferrin receptor was monitored using 1:5000 diluted anti-human IgG-HRP conjugate (Jackson Laboratories) and TMVB substrate reagent. As shown in FIG. 3, CYC17 mutants with amino acid substitutions W9H, L11H, S13H, W14H and G15H exhibited reduced affinity for transferrin receptor.

TABLE E2
CYC17 Mutants

| | | | SEQ | | SEQ |
|---|---|---|---|---|---|
| | | Peptide | ID | | ID |
| Name | Mutation | sequence | NO | Full Cyclotide Sequence | NO |
| CYC17 | — | LAHKWHLQS | 26 | GGVCLAHKWHLQSWGTCRRD | 72 |
| | | WGT | | SDCPGACICRGNGYCGSGSD | |
| CYC17- | L5H | HAHKWHLQS | 117 | GGVCHAHKWHLQSWGTCRR | 105 |
| 5H | | WGT | | DSDCPGACICRGNGYCGSGSD | |
| CYC17- | A6H | LHHKWHLQS | 118 | GGVCLHHKWHLQSWGTCRRD | 106 |
| 6H | | WGT | | SDCPGACICRGNGYCGSGSD | |
| CYC17- | H7A | LAAKWHLQS | 119 | GGVCLAAKWHLQSWGTCRRD | 107 |
| 7A | | WGT | | SDCPGACICRGNGYCGSGSD | |
| CYC17- | H7A | LAAKWALQS | 120 | GGVCLAAKWALQSWGTCRRD | 108 |
| 7A/10A | H10A | WGT | | SDCPGACICRGNGYCGSGSD | |
| CYC17- | K8H | LAHKWHLQS | 121 | GGVCLAHKWHLQSWGTCRRD | 109 |
| 8H | | WGT | | SDCPGACICRGNGYCGSGSD | |
| CYC17- | W9H | LAHKHHLQS | 122 | GGVCLAHKHHLQSWGTCRRD | 110 |
| 9H | | WGT | | SDCPGACICRGNGYCGSGSD | |
| CYC17- | H10A | LAHKWALQS | 123 | GGVCLAHKWALQSWGTCRRD | 111 |
| 10A | | WGT | | SDCPGACICRGNGYCGSGSD | |
| CYC17- | L11H | LAHKWHHQS | 124 | GGVCLAHKWHHQSWGTCRR | 112 |
| 11H | | WGT | | DSDCPGACICRGNGYCGSGSD | |
| CYC17- | Q12H | LAHKWHLHS | 125 | GGVCLAHKWHLHSWGTCRRD | 113 |
| 12H | | WGT | | SDCPGACICRGNGYCGSGSD | |
| CYC17- | S13H | LAHKWHLQH | 126 | GGVCLAHKWHLQHWGTCRR | 114 |
| 13H | | WGT | | DSDCPGACICRGNGYCGSGSD | |
| CYC17- | W14H | LAHKWHLQS | 127 | GGVCLAHKWHLQSHGTCRRD | 115 |
| 14H | | HGT | | SDCPGACICRGNGYCGSGSD | |
| CYC17- | G15H | LAHKWHLQS | 55 | GGVCLAHKWHLQSWHTCRRD | 101 |
| 15H | | WHT | | SDCPGACICRGNGYCGSGSD | |
| CYC17- | T16H | LAHKWHLQS | 128 | GGVCLAHKWHLQSWGHCRR | 116 |
| 16H | | WGH | | DSDCPGACICRGNGYCGSGSD | |

### Example 6: Assessment of Binding Affinity of Aducanumab and Adalimumab Antibody-Cyclotide Fusions

Monovalent, bivalent and bispecific antibody-cyclotide fusions were made by knob (K) and hole (H) engineering of the Fc domain.

The heavy chain of the antibody aducanumab (Aduhelm™; SEQ ID NO: 129) was engineered by mutation of the CH3 to contain either a knob mutation (K; heavy chain-K set forth in SEQ ID NO: 130) mutation or a hole mutation (H; heavy chain-H set forth in SEQ ID NO:131). The heavy chain-K of aducanumab was fused to cyclotide CYC27 (SEQ ID NO:82) for IGF1R-targeting. The heavy chain-K or the heavy chain-H of aducanumab was fused to cyclotide CYC17 (SEQ ID NO:72) for hTfR-targeting. Nucleic acids encoding the heavy chain-K and heavy-H fusions were introduced with the nucleic acid encoding the light chain of aducanumab (SEQ ID NO:132) into HEK293F for expression and production of monovalent and bispecific heterodimeric aducanumab-cyclotide fusions. A schematic of monovalent and bispecific heterodimeric aducanumab-cyclotide fusions is depicted in FIGS. 1B-1D.

The heavy chain of the antibody adalimumab (Humira™; SEQ ID NO:102) was fused to cyclotide CYC17 for hTfR-targeting as described above. The expression constructs for the adalimumab heavy chain-CYC17 cyclotide fusion and the adalimumab light chain (SEQ ID NO:103) were used to co-transfect HEK293F cells for expression and production of a bivalent adalimumab-cyclotide fusion. A schematic of the bivalent adalimumab-CYC17 cyclotide fusion is depicted in FIG. 1A.

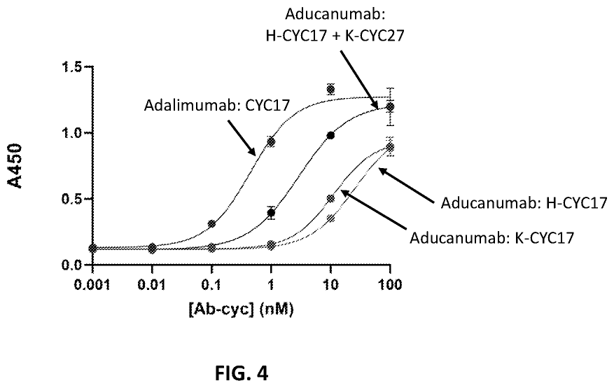

Standard receptor-substrate binding assays showed that the monovalent variants of the aducanumab-CYC17 cyclotide fusions have reduced affinity for hTfR while the bivalent adalimumab-CYC17 cyclotide fusion and the bispecific aducanumab-H-CYC17-K-CYC27 bind to hTfR with higher affinities. Results for binding affinity is shown in FIG. 4. The EC50 binding affinity is depicted in Table E3. Without wishing to be bound by theory, it is believed that a higher affinity antibody-cyclotide fusion may not be released from the endosome following RMT, whereas a lower affinity antibody-cyclotide fusion would be released from the endosome and could be transported to the brain after interaction with the receptor at the blood-brain barrier.

TABLE E3
Affinity for TfR

| Ab-Ab-Cyc | EC50 |
|---|---|
| adalimumab-CYC17 | 0.45 nM |
| Aducanumab-K-CYC17 | 11.20 nM |
| Aduhelm-H-CYC17 | 26.15 nM |
| aducanumab-H-CYC17-K-CYC27 | 2.92 nM |

### Example 7: Generation and Assessment of Monovalent and Bispecific Trastuzumab Antibody-Cyclotide Fusions

Monovalent and bispecific antibody-cyclotide fusions were generated by fusion with the exemplary antibody trastuzumab in which the cyclotides were also fused to the antibody with a flexible linker (SEQ ID NO: 104) with or without a cathepsin B cleavage sequence (CB; SEQ ID NO:133; Taha T A et al. FEBS Lett. 2006 Nov. 13; 580(26):6047-54). The inclusion of a cleavable cathepsin B cleavage site between the linker and cyclotide was added to promote cleavage of the cyclotide peptide portion from the fusion protein after its internalization into the endosome to enhance antibody release from the endosome and transport of the antibody across the blood-brain barrier.

The heavy chain of the antibody trastuzumab (Herceptin®, SEQ ID NO: 134) was engineered by mutation of the CH3 to contain either a knob mutation (K; heavy chain-K set forth in SEQ ID NO: 135) or a hole mutation (H; heavy chain-H set forth in SEQ ID NO:136). The heavy chain-K or the heavy chain-H of trastuzumab was fused to cyclotide CYC17 (SEQ ID NO:72) or the lower affinity mutant CYC17-15H (SEQ ID NO:101) for human transferrin receptor (hTfR)-targeting. For each, the cyclotide was linked to the C-terminal end of the heavy chain-K (SEQ ID NO:135) of trastuzumab via a Gly-Ser linker (e.g. SEQ ID NO: 104), and, in some cases further including the cathepsin B cleavage sequence (SEQ ID NO:133). For bispecific constructs, the heavy chain-K of trastuzumab was fused to cyclotide CYC27 (SEQ ID NO:82) for IGF1R-targeting.

Table E4 sets forth the sequence of generated constructs.

TABLE E4
Trastuzumab Cyclotide Fusions

| | | | | Light | Heavy Chain | | | |
|---|---|---|---|---|---|---|---|---|
| Name | | structure | | chain | IgH | Linker | Cyclotide | Full heavy chain |
| A | Trastuzumab | homodimeric | | 137 | 134 | — | — | 134 |
| | (control) | | | | | | | |
| C | Trastuzumab | heterodimeric | Chain | 137 | 136 | — | — | 136 |
| | CYC17_Ig; | | 1 | | (hole) | | | |
| | monovalent | | Chain | | 135 | 104 | 72 | 139 |
| | | | 2 | | (knob) | (GS) | | |
| D | Trastuzumab | heterodimeric | Chain | 137 | 136 | — | — | 136 |
| | CYC17- | | 1 | | (hole) | | | |
| | CB_Ig; | | Chain | | 135 | 104 | 72 | 140 |
| | monovalent | | 2 | | (knob) | (GS) | | |
| | | | | | | 133 | | |
| | | | | | | (CB) | | |
| E | Trastuzumab | heterodimeric | Chain | 137 | 136 | — | — | 136 |
| | CYC17- | | 1 | | (hole) | | | |
| | 15H_Ig; | | Chain | | 135 | 104 | 101 | 141 |
| | monovalent | | 2 | | (knob) | (GS) | | |
| F | Trastuzumab | heterodimeric | Chain | 137 | 136 | — | — | 136 |
| | CYC17- | | 1 | | (hole) | | | |
| | 15H-CB_Ig; | | Chain | | 135 | 104 | 101 | 142 |
| | monovalent | | 2 | | (knob) | (GS) | | |
| | | | | | | 133 | | |
| | | | | | | (CB) | | |
| G | Trastuzumab | heterodimeric | Chain | 137 | 136 | 104 | 82 | 143 |
| | CYC17- | | 1 | | (hole) | (GS) | | |
| | CB_Ig & | | | | | 133 | | |
| | CYC27- | | | | | (CB) | | |
| | CB_Ig; | | Chain | | 135 | 104 | 72 | 140 |
| | bispecific | | 2 | | (knob) | (GS) | | |
| | | | | | | 133 | | |
| | | | | | | (CB) | | |
| H | Trastuzumab | heterodimeric | Chain | 137 | 136 | 104 | 82 | 143 |
| | CYC17- | | 1 | | (hole) | (GS) | | |
| | 15H-CB_Ig | | | | | 133 | | |
| | & CYC27- | | | | | (CB) | | |
| | CB_Ig; | | Chain | | 135 | 104 | 101 | 142 |
| | bispecific | | 2 | | (knob) | (GS) | | |
| | | | | | | 133 | | |
| | | | | | | (CB) | | |

Nucleic acids encoding the respective full heavy chain-K (e.g., SEQ ID NOS: 139, 140, 141 or 142) and full heavy-H fusions (e.g., SEQ TD NOS: 136 or 143) were introduced with the nucleic acid encoding the light chain of trastuzumab (SEQ TD NO: 137) into HEK293F for expression and production of monovalent and bispecific heterodimeric trastuzumab-cyclotide fusions. For the trastuzumab control, nucleic acids encoding the wild-type (WT) trastuzumab heavy chain (SEQ ID NO: 134) and the light chain of trastuzumab (SEQ ID NO:137) were introduced into HEK293F cells. Cells were grown for 4 to 6 days at 37° C., 8% CO₂, 130 rpm shaking. After removal of cells and filtration of the supernatant, the resulting trastuzumab-cyclotide fusion proteins were purified by Protein A affinity chromatography and concentrated.

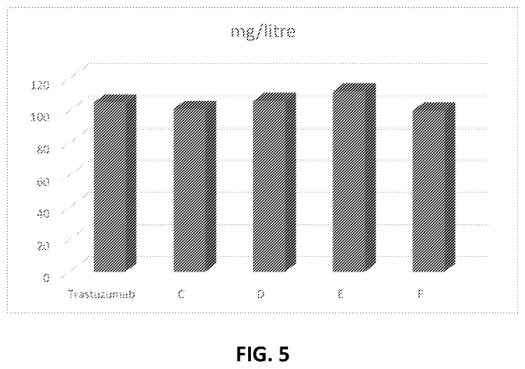

Results for expression of exemplary constructs is shown in FIG. 5. Trastuzumab fusion proteins (C, D, E and F in Table E4) expressed at similar levels to the parental antibody (A in Table E4) as determined by ELISA, with no evidence of aggregation by SDS-PAGE gel electrophoresis.

### Example 8: Glucocerebrosidase Fusion Cyclotide Expression and Activity

The exemplary human transferrin receptor (hTfR) binding cyclotide CYC17 (SEQ ID NO:72) was fused to the enzyme glucocerebrosidase (GlcCSase; SEQ ID NO:144), an enzyme that cleaves by hydrolysis the beta-glucosidic linkage of the chemical glucocerebroside. The cyclotide was linked to the C-terminal end of glucocerebrosidase via a Gly-Ser linker (e.g. SEQ ID NO: 104). The nucleic acid sequence encoding the CYC17-glucocerebrosidase fusion cyclotide, set forth in SEQ ID NO:146, was expressed in HEK293 cells.

The expression and integrity of the glucocerebrosidase-CYC17 fusion was evaluated by western blot (data not shown) using an anti-glucocerebrosidase antibody. Western blot analysis showed expression of a 61.1 kDa protein corresponding to the size of the GlcCSase-CYC17 fusion cyclotide (data not shown).

### Glucocerebroside Activity Assay

A standard glucocerebroside activity assay based on the ability of the GlcCSase-CYC17 fusion cyclotide to react with substrate was performed to assess the functional integrity/activity of the fusion cyclotide. Specifically, the enzymatic activity was assayed using the SensoLyte Red Glucocerebrosidase Fluorometric Assay Kit (AnaSpec) according to the manufacturer's protocol. The assay uses a resorufin-β-glucoside substrate that, upon cleavage of the glycosidic linkage by hGCSase, releases resorufin, which fluoresces at 610 nm upon excitation at 570 nm. The kinetic assay was conducted in triplicate in an opaque 96-well plate at room temperature in a SpectraMax fluorescence plate reader over the course of 1 hour using 10 μL of filtered, concentrated supernatant from the HEK293F hGCSase-CYC17 expression culture, 40 μL of assay buffer, and 50 μL of substrate solution. Positive (GCSase enzyme), inhibitor, vehicle only, and substrate only controls were assayed parallelly in triplicate.

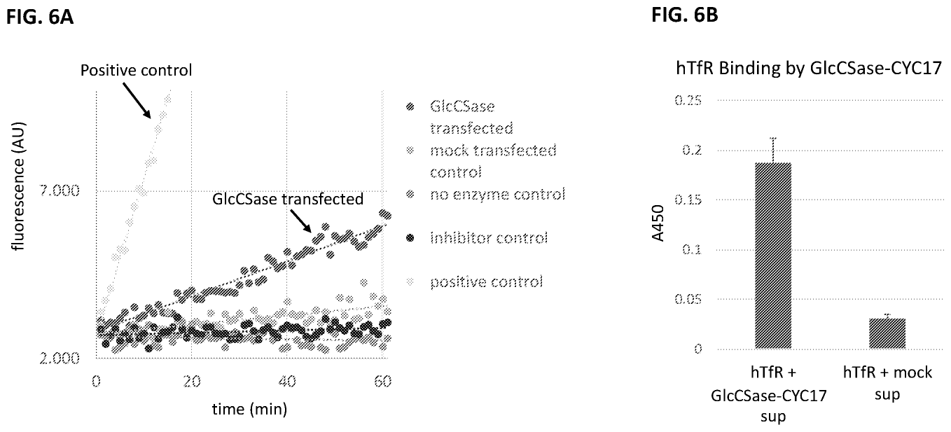

As shown in FIG. 6A, after incubating with the substrate, the GlcCSase-CYC17 fusion cyclotide showed a time dependent increase of emitted fluoresce, which is indicative of enzymatic activity, higher than the levels corresponding to the negative controls.

### Binding to Transferrin Receptor

Binding of the GlcCSase-CYC17 fusion cyclotide was assessed by Enzyme-linked immunosorbent assay (ELISA) of hGCSase-HT2 using human transferrin receptor (hTfR) antigen. Duplicate wells of a high binding half area 96-well plate (Corning) were directly coated with 125 ng of hTfR in 50 μL Tris buffered saline plus 0.1% (v/v) Tween 20 (TBST) containing 2% milk powder (or TBST containing 2% milk powder alone as a negative control) at 4° C. overnight. After removal of the supernatants, the wells are blocked with 50 μL of TBST containing 2% milk powder at room temperature for 2 hours. The blocking agent was removed, and 25 μL of filtered, concentrated supernatant from the HEK293F hGCSase-CYC17 fusion expression culture (or 25 μL of filtered, concentrated supernatant from a non-hGCSase expressing HEK293F expression culture as a negative control) plus 25 μL of TBST containing 4% milk powder were added to the appropriate wells and incubated at room temperature for 1 hour. The supernatants were removed, and wells were washed 4 times with TBST. A ˜1 mg/mL rabbit anti-GCSase primary antibody (Sigma) was diluted 1:300 in TBST containing 2% milk powder, 50 μL is added to each well, and the plate was incubated a room temperature for 1 hour. The supernatants were removed, and the wells were washed 4 times with TBST. A ˜1 mg/mL goat anti-rabbit IgG-HRP secondary antibody was diluted 1:5,000 in TBST containing 2% milk powder, 50 μL is added to each well, and the plate was incubated a room temperature for 30 minutes. The supernatants were removed, and wells were washed 4 times with TBST followed by one time with Tris buffered saline (TBS). 50 μL of tetramethylbenzidine (TMB) substrate was added to each well and incubated at room temperature for 5 minutes, then quenched with 50 μL of 1 N sulfuric acid, and absorbance was read at 450 nm using a SpectraMax absorbance plate reader. GlcCSase-CYC17 fusion cyclotide bound to hTfR as shown in FIG. 6B.

### Example 9: Preparation of NGF Cyclotide Fusions

The exemplary human transferrin receptor (hTfR) binding cyclotide CYC17 (SEQ ID NO:72) was fused to the C-terminal end of nerve growth factor (NGF). Genetic constructs for expression of therapeutic human NGF-cyclotide fusions were prepared by cloning the cyclotide gene fused to the 3′ end of the human Pro-NGF gene (encoding an amino acid sequence set forth in SEQ ID NO:147) via a sequence encoding a flexible linker. The construct included a 5′ IL2 secretory signal peptide sequence for expression. The sequence of the Pro-NGF-CYC17 fusion construct encoded an amino acid sequence set forth in SEQ ID NO:149. The genetic construct also included a nucleotide sequence that encoded for a V5 epitope tag (SEQ ID NO:150) and 6-His tag (SEQ ID NO:151) at the 3′-end of the cyclotide. The genetic construct was cloned into a mammalian expression vector. An exemplary nucleic acid sequence encoding the NGF-CYC17 fusion cyclotide sequence is set forth in SEQ ID NO:153.

For expression, T75 flasks were seeded with 15 mL of HEK 293 EBNA cells at 8×10⁵ cells/ml in Optimem medium (Invitrogen) and incubated over night at 37° C. in a humidified incubator supplemented with 5% CO₂. 180 μL of purified plasmid DNA encoding the cyclotide fusion at 1.0 mg/mL in sterile water was added to 600 μL of Optimem. 66 μL Lipofectamine 2000 (Invitrogen) was added to 660 μL Optimem, mixed briefly and incubated at room temperature for 5 minutes. The two solutions were then combined and incubated for a further 20 minutes at room temperature. Medium from T75 flasks was removed carefully and replaced with 15 mL fresh Optimem. DNA/Lipofactamine/Optimem solution was added to the flasks, which were then placed in at 37° C. in a humidified incubator supplemented with 5% CO₂. Medium was harvested after 7 days and His-tagged proteins were purified via immobilised Metal Anion Chromatography (IMAC).

Harvested medium was centrifuged at 4000 g to remove particulate matter. Imidazole was added to the supernatant to a final concentration of 10 mM. 300 μL of His-Select Nickel Affinity gel beads (Sigma) were added to approximately 30 mL of medium and incubated overnight on a blood mixer. Beads were centrifuged briefly and supernatant was removed. Beads were washed with 1 mL of PBS supplemented with 10 mM imidazole. Beads were centrifuged briefly and supernatant was removed. Beads were washed twice more. Bound proteins were then eluted via the addition of 0.5 mL PBS supplemented with 250 mM imidazole. Imidazole was removed via dialysis in 2 L PBS (pH 7.4) overnight at room temperature, using Slidealyzer dialysis cassettes (10,000 MW cut-off) (Thermo Fisher Scientific Ltd).

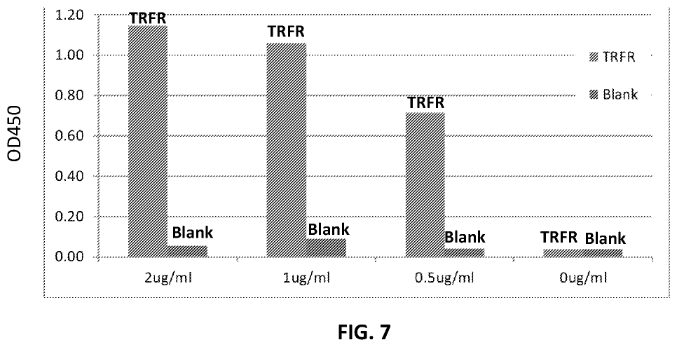

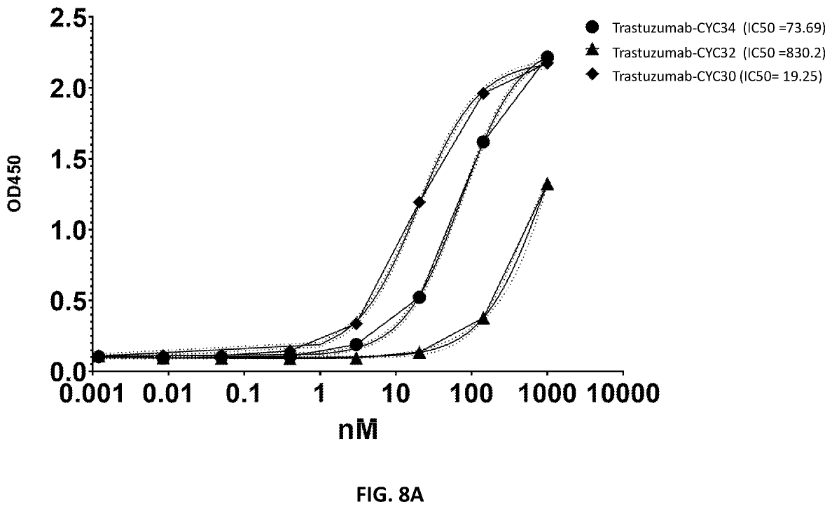

Purified NGF-cyclotide fusion was contacted with His-tagged human transferrin receptor (TrFR) coated at various concentrations (0.5 μg/mL, 1 μg/mL, or 2 μg/mL) on a microwell plate. Binding of the NGF-cyclotide fusion to the coated human transferrin receptor was assessed by ELISA by anti-V5-HRP detection of the NGF-CYC17 fusion protein. As shown in FIG. 7, the NGF-cyclotide fusion bound to hTrFR.

### Example 10: In Vivo Cyclotide-Mediated Delivery of Antibodies into Mouse Brains

Studies were carried out to assess the ability of cyclotides to mediate delivery of antibodies in vivo by receptor-mediated transcytosis (RMT) receptor targeting to the brain. In this example, delivery of the exemplary antibody trastuzumab was assessed following in vivo administration of cyclotide fusion proteins. Monovalent trastuzumab cyclotide fusion proteins were generated by knob (K) and hole (H) engineering of the Fc domain substantially as described in Examples above.

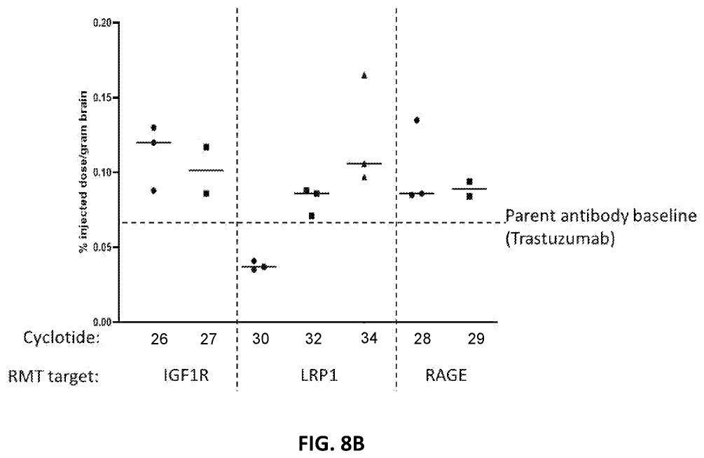

Table E5 sets forth the exemplary tested cyclotide fusions and trastuzumab control. Among the tested cyclotide fusions were fusion conjugates that exhibited different binding affinities for the BBB-R. In an exemplary experiment, heterodimeric cyclotide trastuzumab fusions of CYC30 (SEQ ID NO:85), CYC32 (SEQ ID NO:82) and CYC34 (SEQ ID NO:89) were assessed for binding affinity to human LRP1 by ELISA. As shown in FIG. 8A, the IC50 for binding h LPR1 was 19.25 nM for CYC30 antibody fusion, 73.69 nM for CYC34 antibody fusion, and 830.2 nM for CYC32 antibody fusion.

TABLE E5
Trastuzumab Cyclotide Fusions

| | Cyclotide | Cyclotide | | Light chain | Heavy Chain | |
|---|---|---|---|---|---|---|
| Name | Target | (SEQ ID NO) | Structure | (SEQ ID NO) | (SEQ ID NO) | |
| Trastuzumab | — | | homodimeric | 137 | | 134 |
| (control) | | | | | | |
| Trastuzumab | Hu IgFR | 81 | heterodimeric | 137 | Chain 1: | 178 |
| CYC26_Ig; | | | | | hole | |
| monovalent | | | | | Chain 2: | 179 |
| | | | | | knob | |
| Trastuzumab | Hu IgFR | 82 | heterodimeric | 137 | Chain 1: | 178 |
| CYC27_Ig; | | | | | knob | |
| monovalent | | | | | Chain 2: | 180 |
| | | | | | hole | |
| Trastuzumab | Hu LRP-1 | 85 | heterodimeric | 137 | Chain 1: | 178 |
| CYC30-Ig; | | | | | knob | |
| monovalent | | | | | Chain 2: | 181 |
| | | | | | hole | |
| Trastuzumab | Hu LRP-1 | 87 | heterodimeric | 137 | Chain 1: | 178 |
| CYC32_Ig; | | | | | knob | |
| monovalent | | | | | Chain 2: | 182 |
| | | | | | hole | |
| Trastuzumab | Hu LRP-1 | 89 | heterodimeric | 137 | Chain 1: | 178 |
| CYC34 _Ig; | | | | | knob | |
| monovalent | | | | | Chain 2: | 183 |
| | | | | | hole | |
| Trastuzumab | Hu RAGE | 83 | heterodimeric | 137 | Chain 1: | 178 |
| CYC28 _Ig; | | | | | knob | |
| monovalent | | | | | Chain 2: | 184 |
| | | | | | hole | |
| Trastuzumab | Hu RAGE | 84 | heterodimeric | 137 | Chain 1: | 178 |
| CYC29 _Ig; | | | | | knob | |
| monovalent | | | | | Chain 2: | 185 |
| | | | | | hole | |

Female C57BL/6 mice, age 10-12 weeks, were weighed and administered purified trastuzumab antibody or trastuzumab antibody-monovalent cyclotide fusions, made monovalent by knobs-holes fusion (targeting IGF1R, LRP1, or RAGE receptors) in triplicate at 25 mg/kg in phosphate buffered saline (PBS). After 24 hours, mice were perfused with Dulbecco's PBS containing anesthetic via aortic puncture and brains were removed. One brain hemisphere was transferred to 4% paraformaldehyde in PBS for 24 hours at 4° C., then transferred to PBS at 4° C., paraffin embedded, thin-sectioned using a microtome, and slices were mounted on slides for immunohistochemistry (IHC). The other brain hemisphere was flash frozen and stored at −80° C. prior to homogenization.

For brain homogenization, half brains were weighed and transferred to protease cocktail-treated bead beating homogenization tubes (Bertin Corp.). To each half brain was added 1 mL of PBS containing 1% NP-40, 5 mM EDTA; and tissue was disrupted using a bead beating homogenizer (Bertin Corp.) via 2×30 second pulses separated by a 15 second pause at 6,500 rpm. Brain homogenates were rotated at 4° C. for 1 hour, then centrifuged at 14,000 rpm, 4° C. for 20 minutes. Clarified homogenates (parenchyma) were carefully transferred to an Eppendorf tube and stored at −80° C.

Antibodies present in brain were quantified by enzyme-linked immunosorbent assay (ELISA). Clear half-area high binding 96-well plates were coated with 250 ng/well donkey anti-human Fc IgG overnight at 4° C. (Jackson). Liquid was removed, wells were blocked with Tris-buffered saline containing 0.1% Tween 20 (TBST) containing 2% (w/v) milk powder at room temperature for 1 hour, and liquid was again removed. Neat and serially diluted clarified brain homogenates and serial dilutions of each antibody standard were prepared in TBST containing 2% (w/v) milk powder, added in duplicate to plates, and incubated for 1 hour at room temperature. Plates were washed 4 times with TBST and treated with horseradish peroxidase (HRP)-conjugated goat anti-human Fc IgG (1:5000, Jackson) in TBST containing 2% (w/v) milk powder at room temperature for 30 minutes. Plates were washed 4 times with TBST and once with TBS, developed with 3,3′,5,5′-tetramethylbenzidine (TMB) for 2 minutes, quenched with 1 N sulfuric acid, and read at 450 nm in a SpectraMax plate reader. A standard curve for each antibody was generated from ELISA data by nonlinear regression using GraphPad Prism data analysis software. Amounts of antibodies (e.g., antibody fusion proteins or antibody alone) present in brain homogenates were determined by interpolation from each standard curve and were expressed as percents of injected dose per gram of brain (FIG. 8B).

Cyclotides targeting LRP1, IGF1R and RAGE enhanced trastuzumab delivery compared with unlabelled trastuzumab. These data demonstrate that cyclotide fusions targeting RMT receptors improve brain-specific delivery of a therapeutic agent. Among the cyclotides targeting LRP1, the CYC34 that had an IC50 of 73.69 nM for binding LPR-1 exhibited the greatest enhancement in trastuzumab delivery to the brain, whereas the CYC30 cyclotide with an IC50 of about1 19.25 nM did not exhibit enhancement. Without wishing to be bound by theory, these results indicate that cyclotide fusion conjugates with an intermediate affinity of 50 nM to 800 nM, such as about 100 nM, are most ideal for delivery of conjugated agents to the brain. These data support the use of antibody-cyclotide fusion proteins for improved delivery of therapeutics to the brain.

### Example 11: Transferrin Receptor Targeting for In Vivo Cyclotide-Mediated Delivery of Antibodies into Mouse Brains

Studies were carried out to assess the ability of a transferrin receptor-targeting cyclotide to mediate delivery of antibodies to the brain. In this example, delivery of the exemplary antibody trastuzumab was assessed following in vivo administration of a transferrin-targeted cyclotide fusion proteins. Monovalent trastuzumab cyclotide fusion proteins were generated by knob (K) and hole (H) engineering of the Fc domain substantially as described in Examples above. Table E6 sets forth the exemplary tested cyclotide fusions and trastuzumab control.

TABLE E6
Trastuzumab- Transferin Targeting Cyclotide Fusions

| | Cyclotide | Cyclotide | | Light chain | Heavy Chain | |
|---|---|---|---|---|---|---|
| Name | Target | (SEQ ID NO) | Structure | (SEQ ID NO) | (SEQ ID NO) | |
| Trastuzumab | — | | homodimeric | 137 | | 134 |
| (control) | | | | | | |
| Trastuzumab | Hu TrfR | 72 | heterodimeric | 137 | Chain 1: | 178 |
| HT2 (CYC17); | | | | | hole | |
| monovalent | | | | | Chain 2: | 139 |
| | | | | | knob | |
| Trastuzumab | Hu TrFR | 101 | heterodimeric | 137 | Chain 1: | 178 |
| HT2G15H | | | | | knob | |
| (CYC1715H); | | | | | Chain 2: | 141 |
| monovalent | | | | | hole | |

Female C57BL/6 mice, age 10-12 weeks, were weighed and administered purified trastuzumab antibody or trastuzumab antibody-cyclotide fusions (monovalent CYC17 or CYC17-15H mutant cyclotide paired in knobs-holes format) in triplicate at 25 mg/kg in phosphate buffered saline (PBS). After 24 hours mice were perfused with Dulbecco's PBS containing anesthetic via aortic puncture and brains were removed. One brain hemisphere was transferred to 4% paraformaldehyde in PBS for 24 hours at 4° C., then transferred to PBS at 4° C., paraffin embedded, thin-sectioned using a microtome, and slices were mounted on slides for immunohistochemistry (IHC). The other brain hemisphere was flash frozen and stored at −80° C. prior to homogenization.

For brain homogenization, half brains were weighed and transferred to protease cocktail-treated bead beating homogenization tubes (Bertin Corp.). To each half brain was added 1 mL of PBS containing 1% NP-40, 5 mM EDTA; and tissue was disrupted using a bead beating homogenizer (Bertin Corp.) via 2×30 second pulses separated by a 15 second pause at 6,500 rpm. Brain homogenates were rotated at 4° C. for 1 hour, then centrifuged at 14,000 rpm, 4° C. for 20 minutes. Clarified homogenates (parenchyma) were carefully transferred to an Eppendorf tube and stored at −80° C.

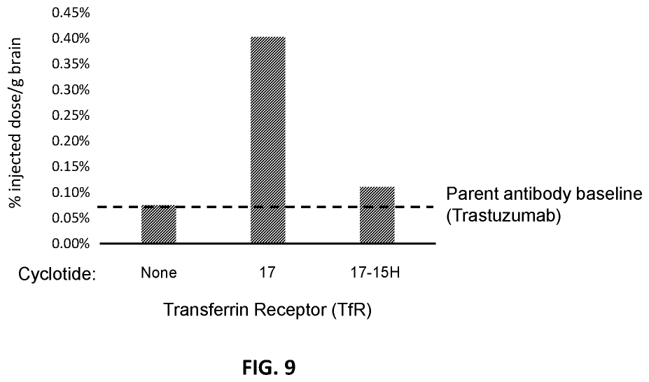

Antibodies present in brain were quantified by enzyme-linked immunosorbent assay (ELISA). Clear half-area high binding 96-well plates were coated with 250 ng/well donkey anti-human Fc IgG overnight at 4° C. (Jackson). Liquid was removed, wells were blocked with Tris-buffered saline containing 0.1% Tween 20 (TBST) containing 2% (w/v) milk powder at room temperature for 1 hour, and liquid was again removed. Neat and serially diluted clarified brain homogenates and serial dilutions of each antibody standard were prepared in TBST containing 2% (w/v) milk powder, added in duplicate to plates, and incubated for 1 hour at room temperature. Plates were washed 4 times with TBST and treated with horseradish peroxidase (tRP)-conjugated goat anti-human Fc IgG (1:5000, Jackson) in TBST containing 2% (w/v) milk powder at room temperature for 30 min. Plates were washed 4 times with TBST and once with TBS, developed with 3,3′,5,5′-tetramethylbenzidine (TMB) for 2 minutes, quenched with 1 N sulfuric acid, and read at 450 nm in a SpectraMax plate reader. A standard curve for each antibody was generated from ELISA data by nonlinear regression using GraphPad Prism data analysis software. Amounts of antibodies (e.g., antibody fusion proteins or antibody alone) present in brain homogenates were determined by interpolation from each standard curve and were expressed as percents of injected dose per gram of brain (FIG. 9).

These data demonstrate that the transferrin targeting cyclotides, particularly CYC17, improved brain-specific delivery of a therapeutic agent. These data support the use of antibody-cyclotide fusion proteins for improved delivery of therapeutics to the brain.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | GGVCPKILQRCRRDSDCPGACICRGNGYCGSGSD | MCOTI I |
| 2 | GGVCPKILKKCRRDSDCPGACICRGNGYCGSGSD | MCOTI II |
| 3 | ERACPRILKKCRRDSDCPGACICRGNGYCG | MCOTI III |
| 4 | GGTGGTGTTTGTCCTAAAATTCTCAAAAAATGTCGTCG | MCOTI II |
| | TGATTCTGATTGTCCTGGAGCGTGTATTTGTCGTGGTA | DNA |
| | ATGGTTATTGCGGTTCTGGTTCTGAT | |
| 5 | ATG GCC GGT GGT GTT TGT (NNN)10 TGT CGT CGT | LOOP1FOR10 |
| | GAT TCT GAT TG | N = a or g or c or |
| | | t/u, unknown, or |
| | | other |
| 6 | ATG GCC GGT GGT GTT TGT (NNN)12 TGT CGT CGT | LOOP1FOR12 |
| | GAT TCT GAT TG | N = a or g or c or |
| | | t/u, unknown, or |
| | | other |
| 7 | CAA CTT TCA ACA GTT TCA GCA GAG G | pIIISEQREV |
| 8 | GCC CAG CCG GCC ATG GCC GGT GGT GTT TG | LOOPILIBPT |
| 9 | TTT CTC CTA TGC GGC CGC ATC AGA ACC AGA ACC | LOOP5LIBPTR |
| | GC | EV |
| 10 | PWPKWTYMAQKY | Hu ObR peptide |
| 11 | QSFFWWHSWFGV | Hu ObR peptide |
| 12 | HSQFWPWEWYQF | Hu ObR peptide |
| 13 | WRWTPKIWVLEEMSS | Hu ObR peptide |
| 14 | WQGMMLFFVGEK | Hu ObR peptide |
| 15 | GWFYWYHKWFGP | Hu ObR peptide |
| 16 | YDWGWFWPFEQY | Hu ObR peptide |
| 17 | SDRWDWMFATWL | Hu ObR peptide |
| 18 | VQPWYSWFMEQG | Hu ObR peptide |
| 19 | PTQTWWFWLPAQ | Hu ObR peptide |
| 20 | YVDWPWTLTLWY | Hu ObR peptide |
| 21 | EWSDWFPYEFLF | Hu ObR peptide |
| 22 | PTAMWFDWFTVY | Hu ObR peptide |
| 23 | FLWFWYGFSRTL | Hu ObR peptide |
| 24 | RHSYSQIPLW | Hu ErbB3 |
| | | peptide |
| 25 | WWDKWVFPISRQ | Hu ErbB3 |
| | | peptide |
| 26 | LAHKWHLQSWGT | HuTrfR peptide |
| 27 | DPRWLAGWGS | HuTrfR peptide |
| 28 | TVWPFVWWGGVP | MuTrfR peptide |
| 29 | AYVHDWYHHVMY | MuTrfR peptide |
| 30 | MHNSASHNIQMQ | MuTrfR peptide |
| 31 | QTVFPMWPWYQL | MuTrfR peptide |
| 32 | HNLFVIESILFW | MuTrfR peptide |
| 33 | KALWYMGWGT | HuTrfR peptide |
| 34 | KQNWLPGWGT | HuTrfR peptide |
| 35 | FDPFYAMLQQLV | HuIGFR peptide |
| 36 | QGFYSWFAVQTM | HuIGFR peptide |
| 37 | HVGYWWHIWMGA | Hu RAGE |
| | | peptide |
| 38 | VSNFWFMHWFGV | Hu RAGE |
| | | peptide |
| 39 | WLTLGIANKGIDVYDVK | Hu LRP-1 |
| | | peptide |
| 40 | NPLKLKYWREWTYGGG | Hu LRP-1 |
| | | peptide |
| 41 | YTQNRYKWLYAQTLN | Hu LRP-1 |
| | | peptide |
| 42 | IEDRKHHRNLHQTVQ | Hu LRP-1 |
| | | peptide |
| 43 | WYETKTKLYVGVVK | Hu LRP-1 |
| | | peptide |
| 44 | LALTKPYWQWAM | Hu LRP-1 |
| | | peptide |
| 45 | WHHEWITWWFWH | Hu LRP-1 |
| | | peptide |
| 46 | DQSPYKLKHYGW | Hu LRP-1 |
| | | peptide |
| 47 | PWYKMWFYETHR | Hu LRP-1 |
| | | peptide |
| 48 | TPWKSQPLFWFQ | Hu LRP-1 |
| | | peptide |
| 49 | EYFRWHLDSWGE | Hu TrfR peptide |
| 50 | LVSQWHIESWGQ | Hu TrfR peptide |
| 51 | QEERWHMHSWGP | Hu TrfR peptide |
| 52 | RYQEWHMESWGS | Hu TrfR peptide |
| 53 | LYEAWHLPSWGQ | Hu TrfR peptide |
| 54 | QMVSEHWVARVW | Hu TrfR peptide |
| 55 | LAHKWHLQSWHT | Hu TrfR peptide |
| 56 | GGVCPWPKWTYMAQKYCRRDSDCPGACICRGNGYCGS | CYC1, Hu ObR |
| | GSD | |
| 57 | GGVCQSFFWWHSWFGVCRRDSDCPGACICRGNGYCGSG | CYC2, Hu ObR |
| | SD | |
| 58 | GGVCHSQFWPWEWYQFCRRDSDCPGACICRGNGYCGSG | CYC3, Hu ObR |
| | SD | |
| 59 | GGVCWRWTPKIWVLEEMSSCSDCPGACICRGNGYCGSG | CYC4, Hu ObR |
| | SD | |
| 60 | GGVCWQGMMLFFVGEKCRRDSDCPGACICRGNGYCGS | CYC5, Hu ObR |
| | GSD | |
| 61 | GGVCGWFYWYHKWFGPCRRDSDCPGACICRGNGYCGS | CYC6, Hu ObR |
| | GSD | |
| 62 | GGVCYDWGWFWPFEQYCRRDSDCPGACICRGNGYCGS | CYC7, Hu ObR |
| | GSD | |
| 63 | GGVCSDRWDWMFATWLCRRDSDCPGACICRGNGYCGSG | CYC8, Hu ObR |
| | SD | |
| 64 | GGVCVQPWYSWFMEQGCRRDSDCPGACICRGNGYCGS | CYC9, Hu ObR |
| | GSD | |
| 65 | GGVCPTQTWWFWLPAQCRRDSDCPGACICRGNGYCGSG | CYC10, Hu ObR |
| | SD | |
| 66 | GGVCYVDWPWTLTLWYCRRDSDCPGACICRGNGYCGSG | CYC11, Hu ObR |
| | SD | |
| 67 | GGVCEWSDWFPYEFLFCRRDSDCPGACICRGNGYCGSGS | CYC12, Hu ObR |
| | D | |
| 68 | GGVCPTAMWFDWFTVYCRRDSDCPGACICRGNGYCGSG | CYC13, Hu ObR |
| | SD | |
| 69 | GGVCFLWFWYGFSRTLCRRDSDCPGACICRGNGYCGSGS | CYC14, Hu ObR |
| | D | |
| 70 | GGVCRHSYSQIPLWCRRDSDCPGACICRGNGYCGSGSD | CYC15, Hu |
| | | ErbB3 |
| 71 | GGVCWWDKWVFPISRQCRRDSDCPGACICRGNGYCGSG | CYC16, Hu |
| | SD | ErbB3 |
| 72 | GGVCLAHKWHLQSWGTCRRDSDCPGACICRGNGYCGSG | CYC17, HuTrfR |
| | SD | |
| 73 | GGVCDPRWLAGWGSCRRDSDCPETCICRGNGYCASGYD | CYC18, HuTrfR |
| 74 | GGVCTVWPFVWWGGVPCRRDSDCPGACICRGNGYCGS | CYC19, MuTrfR |
| | GSD | |
| 75 | GGVCAYVHDWYHHVMYCRRDSDCPGACICRGNGYCGS | CYC20, MuTrfR |
| | GSD | |
| 76 | GGVCMHNSASHNIQMQCRRDSDCPGACICRGNGYCGSG | CYC21, MuTrfR |
| | SD | |
| 77 | GGVCQTVFPMWPWYQLCRRDSDCPGACICRGNGYCGSG | CYC22, MuTrfR |
| | SD | |
| 78 | GGVCHNLFVIESILFWCRRDSDCPGACICRGNGYCGSGSD | CYC23, MuTrfR |
| 79 | GGVCKALWYMGWGTCRRDSDCPGACICRGNGYCGSGS | CYC24, HuTrfR |
| | D | |
| 80 | GGVCKQNWLPGWGTCRRDSDCPGACICRGNGYCGSGSD | CYC25, HuTrfR |
| 81 | GGVCFDPFYAMLQQLVCRRDSDCPGACICRGNGYCGSGS | CYC26, |
| | D | HuIGFR |
| 82 | GGVCQGFYSWFAVQTMCRRDSDCPGACICRGNGYCGSG | CYC27, |
| | SD | HuIGFR |
| 83 | GGVCHVGYWWHIWMGACRRDSDCPGACICRGNGYCGS | CYC28, Hu |
| | GSD | RAGE |
| 84 | GGVCVSNFWFMHWFGVCRRDSDCPGACICRGNGYCGSG | CYC29, Hu |
| | SD | RAGE |
| 85 | GGVCWLTLGIANKGIDVYDVKCRRDSDCPGACICRGNG | CYC30, Hu |
| | YCGSGSD | LRP-1 |
| 86 | GGVCNPLKLKYWREWTYGGGCRRDSDCPGACICRGNG | CYC31, Hu |
| | YCGSGSD | LRP-1 |
| 87 | GGVCYTQNRYKWLYAQTLNCRRDSDCPGACICRGNGYC | CYC32, Hu |
| | GSGSD | LRP-1 |
| 88 | GGVCIEDRKHHRNLHQTVQCRRDSDCPGACICRGNGYC | CYC33, Hu |
| | GSGSD | LRP-1 |
| 89 | GGVCWYETKTKLYVGVVKCRRDSDCPGACICRGNGYCG | CYC34, Hu |
| | SGSD | LRP-1 |
| 90 | GGVCLALTKPYWQWAMCRRDSDCPGACICRGNGYCGS | CYC35, Hu |
| | GSD | LRP-1 |
| 91 | GGVCWHHEWITWWFWHCRRDSDCPGACICRGNGYCGS | CYC36, Hu |
| | GSD | LRP-1 |
| 92 | GGVCDQSPYKLKHYGWCRRDSDCPGACICRGNGYCGSG | CYC37, Hu |
| | SD | LRP-1 |
| 93 | GGVCPWYKMWFYETHRCRRDSDCPGACICRGNGYCGS | CYC38, Hu |
| | GSD | LRP-1 |
| 94 | GGVCTPWKSQPLFWFQCRRDSDCPGACICRGNGYCGSGS | CYC39, Hu |
| | D | LRP-1 |
| 95 | GGVCEYFRWHLDSWGECRRDSDCPGACICRGNGYCGSG | CYC40, Hu |
| | SD | TrfR |
| 96 | GGVCLVSQWHIESWGQCRRDSDCPGACICRGNGYCGSG | CYC41, Hu |
| | SD | TrfR |
| 97 | GGVCQEERWHMHSWGPCRRDSDCPGACICRGNGYCGS | CYC42, Hu |
| | GSD | TrfR |
| 98 | GGVCRYQEWHMESWGSCRRDSDCPGACICRGNGYCGS | CYC43, Hu |
| | GSD | TrfR |
| 99 | GGVCLYEAWHLPSWGQCRRDSDCPGACICRGNGYCGSG | CYC44, Hu |
| | SD | TrfR |
| 100 | GGVCQMVSEHWVARVWCRRDSDCPGACICRGNGYCGS | CYC45, Hu |
| | GSD | TrfR |
| 101 | GGVCLAHKWHLQSWHTCRRDSDCPGACICRGNGYCGSG | CYC17-15H, Hu |
| | SD | TrfR, |
| 102 | EVOLVESGGGLVQPGRSLRLSCAASGFTFDDYAMHWVR | Heavy chain |
| | QAPGKGLEWVSAITWNSGHIDYADSVEGRFTISRDNAKN | (adalimumab) |
| | SLYLQMNSLRAEDTAVYYCAKVSYLSTASSLDYWGQGT | |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSC | |
| 103 | DIQMTQSPSSLSASVGDRVTITCRASQGIRNYLAWYQQKP | Light chain |
| | GKAPKLLIYAASTLQSGVPSRFSGSGSGTDFTLTISSLQPE | (adalimumab) |
| | DVATYYCQRYNRAPYTFGQGTKVEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| 104 | GSGGGSGGGGSGGGGS | GS linker |
| 105 | GGVCHAHKWHLQSWGTCRRDSDCPGACICRGNGYCGS | CYC17-5H, Hu |
| | GSD | TrfR |
| 106 | GGVCLHHKWHLQSWGTCRRDSDCPGACICRGNGYCGSG | CYC17-6H, Hu |
| | SD | TrfR, |
| 107 | GGVCLAAKWHLQSWGTCRRDSDCPGACICRGNGYCGSG | CYC17-7A, Hu |
| | SD | TrfR, |
| 108 | GGVCLAAKWALQSWGTCRRDSDCPGACICRGNGYCGSG | CYC17-7A/10A, |
| | SD | Hu TrfR, |
| 109 | GGVCLAHKWHLQSWGTCRRDSDCPGACICRGNGYCGSG | CYC17-8H, Hu |
| | SD | TrfR, |
| 110 | GGVCLAHKHHLQSWGTCRRDSDCPGACICRGNGYCGSG | CYC17-9H, Hu |
| | SD | TrfR, |
| 111 | GGVCLAHKWALQSWGTCRRDSDCPGACICRGNGYCGSG | CYC17-10A, Hu |
| | SD | TrfR, |
| 112 | GGVCLAHKWHHQSWGTCRRDSDCPGACICRGNGYCGS | CYC17-11H, Hu |
| | GSD | TrfR, |
| 113 | GGVCLAHKWHLHSWGTCRRDSDCPGACICRGNGYCGSG | CYC17-12H, Hu |
| | SD | TrfR, |
| 114 | GGVCLAHKWHLQHWGTCRRDSDCPGACICRGNGYCGS | CYC17-13H, Hu |
| | GSD | TrfR, |
| 115 | GGVCLAHKWHLQSHGTCRRDSDCPGACICRGNGYCGSG | CYC17-14H, Hu |
| | SD | TrfR, |
| 116 | GGVCLAHKWHLQSWGHCRRDSDCPGACICRGNGYCGS | CYC17-16H, Hu |
| | GSD | TrfR, |
| 117 | HAHKWHLQSWGT | Hu TrfR peptide |
| 118 | LHHKWHLQSWGT | Hu TrfR peptide |
| 119 | LAAKWHLQSWGT | Hu TrfR peptide |
| 120 | LAAKWALQSWGT | Hu TrfR peptide |
| 121 | LAHKWHLQSWGT | Hu TrfR peptide |
| 122 | LAHKHHLQSWGT | Hu TrfR peptide |
| 123 | LAHKWALQSWGT | Hu TrfR peptide |
| 124 | LAHKWHHQSWGT | Hu TrfR peptide |
| 125 | LAHKWHLHSWGT | Hu TrfR peptide |
| 126 | LAHKWHLQHWGT | Hu TrfR peptide |
| 127 | LAHKWHLQSHGT | Hu TrfR peptide |
| 128 | LAHKWHLQSWGH | Hu TrfR peptide |
| 129 | QVQLVESGGGVVQPGRSLRLSCAASGFAFSSYGMHWVR | Heavy chain |
| | QAPGKGLEWVAVIWFDGTKKYYTDSVKGRFTISRDNSK | (aducanumab) |
| | NTLYLQMNTLRAEDTAVYYCARDRGIGARRGPYYMDV | |
| | WGKGTTVTVSSASTKGPSVFPLAPSSKSTSGGTAALGCL | |
| | VKDYFPEPVTVSWNSGALTSGVHTFPAVLQSSGLYSLSS | |
| | VVTVPSSSLGTQTYICNVNHKPSNTKVDKRVEPKSCDKT | |
| | HTCPPCPAPELLGGPSVFLFPPKPKDTLMISRTPEVTCVVV | |
| | DVSHEDPEVKFNWYVDGVEVHNAKTKPREEQYNSTYRV | |
| | VSVLTVLHQDWLNGKEYKCKVSNKALPAPIEKTISKAKG | |
| | QPREPQVYTLPPSREEMTKNQVSLTCLVKGFYPSDIAVE | |
| | WESNGQPENNYKTTPPVLDSDGSFFLYSKLTVDKSRWQQ | |
| | GNVFSCSVMHEALHNHYTQKSLSLSPG | |
| 130 | QVQLVESGGGVVQPGRSLRLSCAASGFAFSSYGMHWVR | Heavy chain- |
| | QAPGKGLEWVAVIWFDGTKKYYTDSVKGRFTISRDNSK | knob |
| | NTLYLQMNTLRAEDTAVYYCARDRGIGARRGPYYMDV | (aducanumab) |
| | WGKGTTVTVSSASTKGPSVFPLAPSSKSTSGGTAALGCL | |
| | VKDYFPEPVTVSWNSGALTSGVHTFPAVLQSSGLYSLSS | |
| | VVTVPSSSLGTQTYICNVNHKPSNTKVDKRVEPKSCDKT | |
| | HTCPPCPAPELLGGPSVFLFPPKPKDTLMISRTPEVTCVVV | |
| | DVSHEDPEVKFNWYVDGVEVHNAKTKPREEQYNSTYRV | |
| | VSVLTVLHQDWLNGKEYKCKVSNKALPAPIEKTISKAKG | |
| | QPREPQVYTLPPCREEMTKNQVSLWCLVKGFYPSDIAVE | |
| | WESNGQPENNYKTTPPVLDSDGSFFLYSKLTVDKSRWQQ | |
| | GNVFSCSVMHEALHNHYTQKSLSLSPG | |
| 131 | QVQLVESGGGVVQPGRSLRLSCAASGFAFSSYGMHWVR | Heavy chain- |
| | QAPGKGLEWVAVIWFDGTKKYYTDSVKGRFTISRDNSK | hole |
| | NTLYLQMNTLRAEDTAVYYCARDRGIGARRGPYYMDV | (aducanumab) |
| | WGKGTTVTVSSASTKGPSVFPLAPSSKSTSGGTAALGCL | |
| | VKDYFPEPVTVSWNSGALTSGVHTFPAVLQSSGLYSLSS | |
| | VVTVPSSSLGTQTYICNVNHKPSNTKVDKRVEPKSCDKT | |
| | HTCPPCPAPELLGGPSVFLFPPKPKDTLMISRTPEVTCVVV | |
| | DVSHEDPEVKFNWYVDGVEVHNAKTKPREEQYNSTYRV | |
| | VSVLTVLHQDWLNGKEYKCKVSNKALPAPIEKTISKAKG | |
| | QPREPQVCTLPPSREEMTKNQVSLSCAVKGFYPSDIAVE | |
| | WESNGQPENNYKTTPPVLDSDGSFFLVSKLTVDKSRWQQ | |
| | GNVFSCSVMHEALHNHYTQKSLSLSPG | |
| 132 | DIQMTQSPSSLSASVGDRVTITCRASQSISSYLNWYQQKP | light chain |
| | GKAPKLLIYAASSLQSGVPSRFSGSGSGTDFTLTISSLQPE | (aducanumab) |
| | DFATYYCQQSYSTPLTFGGGTKVEIKRTVAAPSVFIFPPSD | |
| | EQLKSGTASVVCLLNNFYPREAKVQWKVDNALQSGNSQ | |
| | ESVTEQDSKDSTYSLSSTLTLSKADYEKHKVYACEVTHQ | |
| | GLSSPVTKSFNRGEC | |
| 133 | LAASINHYAGYEQV | Cathepsin B |
| | | (CB) cleavage |
| | | site |
| 134 | EVQLVESGGGLVQPGGSLRLSCAASGFNIKDTYIHWVRQ | WT igH |
| | APGKGLEWVARIYPTNGYTRYADSVKGRFTISADTSKNT | Trastuzumab |
| | AYLQMNSLRAEDTAVYYCSRWGGDGFYAMDYWGQGT | |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPSRDELTKNQVSLTCLVKGFYPSDIAVEWESNGQP | |
| | ENNYKTTPPVLDSDGSFFLYSKLTVDKSRWQQGNVFSCS | |
| | VMHEALHNHYTQKSLSLSPG | |
| 135 | EVQLVESGGGLVQPGGSLRLSCAASGFNIKDTYIHWVRQ | KNOB igH |
| | APGKGLEWVARIYPTNGYTRYADSVKGRFTISADTSKNT | Trastuzumab |
| | AYLQMNSLRAEDTAVYYCSRWGGDGFYAMDYWGQGT | |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPCRDELTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPG | |
| 136 | EVQLVESGGGLVQPGGSLRLSCAASGFNIKDTYIHWVRQ | HOLE igH |
| | APGKGLEWVARIYPTNGYTRYADSVKGRFTISADTSKNT | Trastuzumab |
| | AYLQMNSLRAEDTAVYYCSRWGGDGFYAMDYWGQGT | |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VCTLPPSRDELTKNQVSLSCAVKGFYPSDIAVEWESNGQP | |
| | ENNYKTTPPVLDSDGSFFLVSKLTVDKSRWQQGNVFSCS | |
| | VMHEALHNHYTQKSLSLSPG | |
| 137 | DIQMTQSPSSLSASVGDRVTITCRASQDVNTAVAWYQQK | Trastuzumab VL |
| | PGKAPKLLIYSASFLYSGVPSRFSGSRSGTDFTLTISSLQPE | |
| | DFATYYCQQHYTTPPTFGQGTKVEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |
| 138 | EVQLVESGGGLVQPGGSLRLSCAASGFNIKDTYIHWVRQ | Trastuzumab- |
| | APGKGLEWVARIYPTNGYTRYADSVKGRFTISADTSKNT | CYC17_IgH |
| | AYLQMNSLRAEDTAVYYCSRWGGDGFYAMDYWGQGT | |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPSRDELTKNQVSLTCLVKGFYPSDIAVEWESNGQP | |
| | ENNYKTTPPVLDSDGSFFLYSKLTVDKSRWQQGNVFSCS | |
| | VMHEALHNHYTQKSLSLSPGSGGGSGGGGSGGGGSLEG | |
| | GVCLAHKWHLQSWGTCRRDSDCPGACICRGNGYCGSGS | |
| | D | |
| 139 | EVQLVESGGGLVQPGGSLRLSCAASGFNIKDTYIHWVRQ | Trastuzumab |
| | APGKGLEWVARIYPTNGYTRYADSVKGRFTISADTSKNT | KNOB |
| | AYLQMNSLRAEDTAVYYCSRWGGDGFYAMDYWGQGT | CYC17_IgH |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPCRDELTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPGSGGGSGGGGSGGGGSLE | |
| | GGVCLAHKWHLQSWGTCRRDSDCPGACICRGNGYCGSG | |
| | SD | |
| 140 | EVQLVESGGGLVQPGGSLRLSCAASGFNIKDTYIHWVRQ | Trastuzumab |
| | APGKGLEWVARIYPTNGYTRYADSVKGRFTISADTSKNT | KNOB CYC17 |
| | AYLQMNSLRAEDTAVYYCSRWGGDGFYAMDYWGQGT | CB linker_IgH |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPCRDELTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPGSGGGSGGGGSGGGGSLEL | |
| | AASINHYAGYEQVGGVCLAHKWHLQSWGTCRRDSDCP | |
| | GACICRGNGYCGSGSD | |
| 141 | EVQLVESGGGLVQPGGSLRLSCAASGFNIKDTYIHWVRQ | Trastuzumab |
| | APGKGLEWVARIYPTNGYTRYADSVKGRFTISADTSKNT | KNOB CYC17- |
| | AYLQMNSLRAEDTAVYYCSRWGGDGFYAMDYWGQGT | 15H_IgH |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPCRDELTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPGSGGGSGGGGSGGGGSLE | |
| | GGVCLAHKWHLQSWHTCRRDSDCPGACICRGNGYCGSG | |
| | SD | |
| 142 | EVQLVESGGGLVQPGGSLRLSCAASGFNIKDTYIHWVRQ | Trastuzumab |
| | APGKGLEWVARIYPTNGYTRYADSVKGRFTISADTSKNT | KNOB CYC17- |
| | AYLQMNSLRAEDTAVYYCSRWGGDGFYAMDYWGQGT | 15H-CB |
| | LVTVSSASTKGPSVFPLAPSSKSTSGGTAALGCLVKDYFP | linker_IgH |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPCRDELTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPGSGGGSGGGGSGGGGSLEL | |
| | AASINHYAGYEQVGGVCLAHKWHLQSWHTCRRDSDCP | |
| | GACICRGNGYCGSGSD | |
| 143 | EVQLVESGGGLVQPGGSLRLSCAASGFNIKDTYIHWVRQ | Trastuzumab |
| | APGKGLEWVARIYPTNGYTRYADSVKGRFTISADTSKNT | HOLE CYC27- |
| | AYLQMNSLRAEDTAVYYCSRWGGDGFYAMDYWGQGT | CBlinker_IgH |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VCTLPPSRDELTKNQVSLSCAVKGFYPSDIAVEWESNGQP | |
| | ENNYKTTPPVLDSDGSFFLVSKLTVDKSRWQQGNVFSCS | |
| | VMHEALHNHYTQKSLSLSPGSGGGSGGGGSGGGGSLEL | |
| | AASINHYAGYEQVGGVCQGFYSWFAVQTMCRRDSDCPG | |
| | ACICRGNGYCGSGSD | |
| 144 | ARPCIPKSFGYSSVVCVCNATYCDSFDPPTFPALGTFSRYE | Glucocerebrosidase |
| | STRSGRRMELSMGPIQANHTGTGLLLTLQPEQKFQKVKG | |
| | FGGAMTDAAALNILALSPPAQNLLLKSYFSEEGIGYNIIRV | |
| | PMASCDFSIRTYTYADTPDDFQLHNFSLPEEDTKLKIPLIH | |
| | RALQLAQRPVSLLASPWTSPTWLKTNGAVNGKGSLKGQ | |
| | PGDIYHQTWARYFVKFLDAYAEHKLQFWAVTAENEPSA | |
| | GLLSGYPFQCLGFTPEHQRDFIARDLGPTLANSTHHNVRL | |
| | LMLDDQRLLLPHWAKVVLTDPEAAKYVHGIAVHWYLD | |
| | FLAPAKATLGETHRLFPNTMLFASEACVGSKFWEQSVRL | |
| | GSWDRGMQYSHSIITNLLYHVVGWTDWNLALNPEGGPN | |
| | WVRNFVDSPIIVDITKDTFYKQPMFYHLGHFSKFIPEGSQ | |
| | RVGLVASQKNDLDAVALMHPDGSAVVVVLNRSSKDVPL | |
| | TIKDPAVGFLETISPGYSIHTYLWHRQ | |
| 145 | ARPCIPKSFGYSSVVCVCNATYCDSFDPPTFPALGTFSRYE | Glucocerebrosidase |
| | STRSGRRMELSMGPIQANHTGTGLLLTLQPEQKFQKVKG | |
| | FGGAMTDAAALNILALSPPAQNLLLKSYFSEEGIGYNIIRV | |
| | PMASCDFSIRTYTYADTPDDFQLHNFSLPEEDTKLKIPLIH | |
| | RALQLAQRPVSLLASPWTSPTWLKTNGAVNGKGSLKGQ | |
| | PGDIYHQTWARYFVKFLDAYAEHKLQFWAVTAENEPSA | |
| | GLLSGYPFQCLGFTPEHQRDFIARDLGPTLANSTHHNVRL | |
| | LMLDDQRLLLPHWAKVVLTDPEAAKYVHGIAVHWYLD | |
| | FLAPAKATLGETHRLFPNTMLFASEACVGSKFWEQSVRL | |
| | GSWDRGMQYSHSIITNLLYHVVGWTDWNLALNPEGGPN | |
| | WVRNFVDSPIIVDITKDTFYKQPMFYHLGHFSKFIPEGSQ | |
| | RVGLVASQKNDLDAVALMHPDGSAVVVVLNRSSKDVPL | |
| | TIKDPAVGFLETISPGYSIHTYLWRRQ | |
| 146 | ARPCIPKSFGYSSVVCVCNATYCDSFDPPTFPALGTFSRYE | |
| | STRSGRRMELSMGPIQANHTGTGLLLTLQPEQKFQKVKG | Glucocerebrosidase- |
| | FGGAMTDAAALNILALSPPAQNLLLKSYFSEEGIGYNIIRV | CYC17 with |
| | PMASCDFSIRTYTYADTPDDFQLHNFSLPEEDTKLKIPLIH | linker |
| | RALQLAQRPVSLLASPWTSPTWLKTNGAVNGKGSLKGQ | |
| | PGDIYHQTWARYFVKFLDAYAEHKLQFWAVTAENEPSA | |
| | GLLSGYPFQCLGFTPEHQRDFIARDLGPTLANSTHHNVRL | |
| | LMLDDQRLLLPHWAKVVLTDPEAAKYVHGIAVHWYLD | |
| | FLAPAKATLGETHRLFPNTMLFASEACVGSKFWEQSVRL | |
| | GSWDRGMQYSHSIITNLLYHVVGWTDWNLALNPEGGPN | |
| | WVRNFVDSPIIVDITKDTFYKQPMFYHLGHFSKFIPEGSQ | |
| | RVGLVASQKNDLDAVALMHPDGSAVVVVLNRSSKDVPL | |
| | TIKDPAVGFLETISPGYSIHTYLWHRQGSGGGSGGGGSGG | |
| | GGSLEGGVCLAHKWHLQSWGTCRRDSDCPGACICRGNG | |
| | YCGSGSD | |
| 147 | PHSESNVPAGHTIPQVHWTKLQHSLDTALRRARSAPAAA | NGF |
| | IAARVAGQTRNITVDPRLFKKRRLRSPRVLFSTQPPREAA | |
| | DTQDLDFEVGGAAPFNRTHRSKRSSSHPIFHRGEFSVCDS | |
| | VSVWVGDKTTATDIKGKEVMVLGEVNINNSVFKQYFFE | |
| | TKCRDSNPVDSGCRGIDSKHWNSYCTTTHTFVKALTMD | |
| | GKQAAWEFIRIDTACVCVLSRKAVR | |
| 148 | GGGGS | GS linker |
| 149 | PHSESNVPAGHTIPQVHWTKLQHSLDTALRRARSAPAAA | NGF-CYC17 |
| | IAARVAGQTRNITVDPRLFKKRRLRSPRVLFSTQPPREAA | cyclotide fusion |
| | DTQDLDFEVGGAAPFNRTHRSKRSSSHPIFHRGEFSVCDS | |
| | VSVWVGDKTTATDIKGKEVMVLGEVNINNSVFKQYFFE | |
| | TKCRDSNPVDSGCRGIDSKHWNSYCTTTHTFVKALTMD | |
| | GKQAAWEFIRIDTACVCVLSRKAVRGGGGSGNSGGVCL | |
| | AHKWHLQSWGTCRRDSDCPGACICRGNGYCGSGSD | |
| 150 | GKPIPNPLLGLDST | V5 epitope tag |
| 151 | HHHHHH | His tag |
| 152 | MYRMQLLSCIALSLALVINS | IL-2 secretory |
| 153 | CCAGGATCCACAGGCGAGCCTCACAGCGAGAGCAATG | signal peptide |
| | TGCCTGCCGGCCACACCATCCCTCAGGTGCACTGGACA | CYC17-NGF- |
| | AAGCTGCAGCACAGCCTGGACACCGCCCTGAGAAGGG | V5-His6 Trunc |
| | CCAGATCTGCTCCTGCCGCCGCTATTGCCGCTAGAGTG | |
| | GCCGGACAGACCCGGAACATCACCGTGGACCCCCGGC | |
| | TGTTCAAGAAGCGGAGACTGAGAAGCCCCAGAGTGCT | |
| | GTTCAGCACCCAGCCCCCTAGAGAGGCCGCCGATACC | |
| | CAGGACCTGGATTTCGAAGTGGGCGGAGCCGCCCCTTT | |
| | CAACCGGACCCACAGAAGCAAGCGGAGCAGCAGCCAC | |
| | CCCATCTTCCACAGAGGCGAGTTCAGCGTGTGCGACAG | |
| | CGTGTCCGTGTGGGTGGGAGATAAGACCACCGCCACC | |
| | GACATCAAGGGCAAAGAAGTGATGGTGCTGGGCGAAG | |
| | TGAACATCAACAACAGCGTGTTCAAGCAGTACTTCTTC | |
| | GAGACAAAGTGCCGGGACAGCAACCCCGTGGACAGCG | |
| | GCTGTAGAGGCATCGACAGCAAGCACTGGAACAGCTA | |
| | CTGCACCACCACACACACCTTCGTGAAGGCCCTGACCA | |
| | TGGACGGAAAGCAGGCCGCCTGGGAGTTCATCAGAAT | |
| | CGACACCGCCTGCGTGTGCGTGCTGAGCAGAAAAGCT | |
| | GTGCGGGGAGGCGGCGGAAGCGGGAATTCGGGTGGTG | |
| | TTTGTCTGGCTCATAAATGGCATCTGCAGTCTTGGGGT | |
| | ACTTGTCGTCGTGATTCTGATTGTCCTGGAGCGTGTATT | |
| | TGTCGTGGTAATGGTTATTGCGGTTCTGGTTCTGATGC | |
| | GGCCGCAGGTAAACCTATCCCGAATCCGCTGCTGGGTC | |
| | TGGACTCTACCCATCATCACCATCATCACTGA | |
| 154 | GGGGSGGGGS | (GGGGS)₂ |
| | | linker |
| 155 | GGGGSGGGGSGGGGS | (GGGGS)₃ |
| | | linker |
| 156 | QPMKRLTLGN | Cathepsin |
| | | cleavable linker |
| 157 | GKPILFFRLK | Cathepsin |
| | | cleavable linker |
| 158 | APISFFELG | Cathepsin |
| | | cleavable linker |
| 159 | GRWPPMGLPWE | Cathepsin |
| | | cleavable linker |
| 160 | GRWHPMGAPWE | Cathepsin |
| | | cleavable linker |
| 161 | HPGGPQ | Cathepsin |
| | | cleavable linker |
| 162 | TFLGGPKPPQRVMFTEDLKLPASF | Cathepsin |
| | | cleavable linker |
| 163 | AVPAVTEGPIPEVLK | Cathepsin |
| | | cleavable linker |
| 164 | LSQDTVSVPCQSASSASALG | Cathepsin |
| | | cleavable linker |
| 165 | KGKVFQEPLFYEAPRSVD | Cathepsin |
| | | cleavable linker |
| 166 | ITYKSNPNRILPDSVD | Cathepsin |
| | | cleavable linker |
| 167 | MSYREAASGNFSLF | Cathepsin |
| | | cleavable linker |
| 168 | NALKFLASLLELPE | Cathepsin |
| | | cleavable linker |
| 169 | AGLTTELFSPVDLN | Cathepsin |
| | | cleavable linker |
| 170 | MQYFSHFIRSGNPN | Cathepsin |
| | | cleavable linker |
| 171 | AQTKLLAVSGPFHY | Cathepsin |
| | | cleavable linker |
| 172 | YPYEFSRKVPTFAT | Cathepsin |
| | | cleavable linker |
| 173 | TNSQLFRRAVLMGG | Cathepsin |
| | | cleavable linker |
| 174 | KKKRKVGLEPGEKP | Cathepsin |
| | | cleavable linker |
| 175 | KRKVGLEPGE | Cathepsin |
| | | cleavable linker |
| 176 | RKVGLEPG | Cathepsin |
| | | cleavable linker |
| 177 | XXXXXHXXSWGx | Transferrin |
| | | receptor peptide |
| | | consensus |
| | | X is any amino |
| | | acid |
| 178 | EVQLVESGGGLVQPGGSLRLSCAASGFNIKDTYIHWVRQ | HOLE igH |
| | APGKGLEWVARIYPTNGYTRYADSVKGRFTISADTSKNT | Trastuzumab |
| | AYLQMNSLRAEDTAVYYCSRWGGDGFYAMDYWGQGT | |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VCTLPPSRDELTKNQVSLSCAVKGFYPSDIAVEWESNGQP | |
| | ENNYKTTPPVLDSDGSFFLVSKLTVDKSRWQQGNVFSCS | |
| | VMHEALHNHYTQKSLSLSPGK | |
| 179 | EVQLVESGGGLVQPGGSLRLSCAASGFNIKDTYIHWVRQ | Trastuzumab |
| | APGKGLEWVARIYPTNGYTRYADSVKGRFTISADTSKNT | KNOB |
| | AYLQMNSLRAEDTAVYYCSRWGGDGFYAMDYWGQGT | CYC26_IgH |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPCRDELTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPGSGGGSGGGGSGGGGSLE | |
| | GGVCFDPFYAMLQQLVCRRDSDCPGACICRGNGYCGSG | |
| | SD | |
| 180 | EVQLVESGGGLVQPGGSLRLSCAASGFNIKDTYIHWVRQ | Trastuzumab |
| | APGKGLEWVARIYPTNGYTRYADSVKGRFTISADTSKNT | KNOB |
| | AYLQMNSLRAEDTAVYYCSRWGGDGFYAMDYWGQGT | CYC27_IgH |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPCRDELTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPGSGGGSGGGGSGGGGSLE | |
| | GGVCQGFYSWFAVQTMCRRDSDCPGACICRGNGYCGSG | |
| | SD | |
| 181 | EVQLVESGGGLVQPGGSLRLSCAASGFNIKDTYIHWVRQ | Trastuzumab |
| | APGKGLEWVARIYPTNGYTRYADSVKGRFTISADTSKNT | KNOB |
| | AYLQMNSLRAEDTAVYYCSRWGGDGFYAMDYWGQGT | CYC30_IgH |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPCRDELTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPGSGGGSGGGGSGGGGSLE | |
| | GGVCWLTLGIANKGIDVYDVKCRRDSDCPGACICRGNG | |
| | YCGSGSD | |
| 182 | EVQLVESGGGLVQPGGSLRLSCAASGFNIKDTYIHWVRQ | Trastuzumab |
| | APGKGLEWVARIYPTNGYTRYADSVKGRFTISADTSKNT | KNOB |
| | AYLQMNSLRAEDTAVYYCSRWGGDGFYAMDYWGQGT | CYC32_IgH |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPCRDELTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPGSGGGSGGGGSGGGGSLE | |
| | GGVCYTQNRYKWLYAQTLNCRRDSDCPGACICRGNGYC | |
| | GSGSD | |
| 183 | EVQLVESGGGLVQPGGSLRLSCAASGFNIKDTYIHWVRQ | Trastuzumab |
| | APGKGLEWVARIYPTNGYTRYADSVKGRFTISADTSKNT | KNOB |
| | AYLQMNSLRAEDTAVYYCSRWGGDGFYAMDYWGQGT | CYC34_IgH |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPCRDELTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPGSGGGSGGGGGGGGSLE | |
| | GGVCWYETKTKLYVGVVKCRRDSDCPGACICRGNGYCG | |
| | SGSD | |
| 184 | EVQLVESGGGLVQPGGSLRLSCAASGFNIKDTYIHWVRQ | Trastuzumab |
| | APGKGLEWVARIYPTNGYTRYADSVKGRFTISADTSKNT | KNOB |
| | AYLQMNSLRAEDTAVYYCSRWGGDGFYAMDYWGQGT | CYC28_IgH |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPCRDELTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPGSGGGSGGGGSGGGGSLE | |
| | GGVCHVGYWWHIWMGACRRDSDCPGACICRGNGYCGS | |
| | GSD | |
| 185 | EVQLVESGGGLVQPGGSLRLSCAASGFNIKDTYIHWVRQ | Trastuzumab |
| | APGKGLEWVARIYPTNGYTRYADSVKGRFTISADTSKNT | KNOB |
| | AYLQMNSLRAEDTAVYYCSRWGGDGFYAMDYWGQGT | CYC29_IgH |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPCRDELTKNQVSLWCLVKGFYPSDIAVEWESNGQ | |
| | PENNYKTTPPVLDSDGSFFLYSKLTVDKSRWQQGNVFSC | |
| | SVMHEALHNHYTQKSLSLSPGSGGGSGGGGSGGGGSLE | |
| | GGVCVSNFWFMHWFGVCRRDSDCPGACICRGNGYCGSG | |
| | SD | |
| 186 | GGSGGSGGSGGS | Linker |
| 187 | GGSGGSGGSGGSGGS | Linker |
| 188 | GGGGGSGGGGGSGGGGGS | Linker |
| 189 | GGSGGGGSGGGGSGGGGS | Linker |

1. A modified cyclotide comprising a peptide that binds to a blood-brain barrier trancytosis receptor (BBB-R), wherein:
the peptide is inserted into or replaces one or more amino acids of at least one loop of the cyclotide scaffold set forth in SEQ ID NO:2, SEQ ID NO:1 or SEQ ID NO: 3, and wherein the peptide is about 2 to 50 amino acid residues; and
the BBB-R is selected from the group consisting of transferrin receptor (TrfR), insulin-like growth factor type 1 receptor (IGFR), Erb-B2 Receptor Tyrosine Kinase 3 (ErbB3), leptin receptor (ObR), low-density lipoprotein receptor-related protein 1 (LRP-1), and receptor for advanced glycation end products (RAGE).

2. A modified cyclotide comprising:
i) a peptide that binds to a blood-brain barrier trancytosis receptor (BBB-R) selected from the group consisting of transferrin receptor (TrfR), insulin-like growth factor type 1 receptor (IGFR), Erb-B2 Receptor Tyrosine Kinase 3 (ErbB3), leptin receptor (ObR), low-density lipoprotein receptor-related protein 1 (LRP-1), and receptor for advanced glycation-end products (RAGE), wherein said peptide has an amino acid sequence of 2 to 50 amino acid residues; and
ii) a cyclotide scaffold comprising the peptide of i),
wherein the modified cyclotide comprises the structure (I): 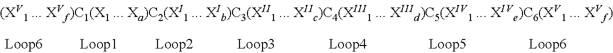
wherein C₁ to C₆ are cysteine residues; wherein each of C₁ and C₄, C₂ and C₅, and C₃ and C₆ are connected by a disulfide bond to form a cysteine knot; wherein each X represents an amino acid residue in a loop, wherein said amino acid residues are the same or different; wherein d is about 1-2; wherein at least one loop from loops 1, 2, 3, 5 or 6 has an amino acid sequence comprising the sequence of said peptide of clause i), wherein any loop comprising said sequence of said peptide of clause i) comprises 2 to about 30 amino acids, and wherein for any of loops 1, 2, 3, 5, or 6 that do not contain said sequence of said peptide of clause i), a, b, c, e, and f, are the same or different, and are each any number from 3-10, and b, c, e, and f are each any number from 1 to 20.

3. The modified cyclotide of claim 1, wherein the cyclotide scaffold is selected from a plant cyclotide.

4. The modified cyclotide of claim 2 or claim 3, wherein the cyclotide scaffold is of the Momordicae species.

5. The modified cyclotide of claim 4, wherein the cyclotide scaffold is a Momordica cochinchinensis trypsin inhibitor.

6. The modified cyclotide of claim 5, wherein the Momordica cochinchinensis trypsin inhibitor is MCoTI-II set forth in SEQ ID NO: 2, MCoTI-I set forth in SEQ ID NO: 1, or MCoTI-III set forth in SEQ ID NO: 3.

7. The modified cyclotide of any of claims 1-6, wherein the BBB-R is human.

8. The modified cyclotide of any of claims 1-7, wherein the sequence of said peptide replaces or substitutes one or more amino acids of one of the one or more loops of the cyclotide scaffold.

9. The modified cyclotide of any of claims 1-8, wherein the cyclotide scaffold is set forth in SEQ ID NO:2.

10. The modified cyclotide of any of claims 1-9, wherein the at least one loop is loop 1, loop 5 or loop 6, or is a combination thereof.

11. The modified cyclotide of any of claims 1-10, wherein the at least one loop is loop 1.

12. The modified cyclotide of any of claims 1-11, wherein the peptide is inserted into and replaces amino acids in only one loop of the cyclotide scaffold.

13. The modified cyclotide of claim 12, wherein the one loop is loop 1.

14. The modified cyclotide of any of claims 11-13, wherein the peptide replaces loop 1 amino acids between cysteine 4 and cysteine 11 of the cyclotide scaffold.

15. A modified cyclotide comprising a peptide inserted into loop 1 to replace all amino acids between cysteine 4 and cysteine 11 of SEQ ID NO:2, wherein the peptide is 2 to 50 amino acid residues and binds to a blood-brain barrier trancytosis receptor (BBB-R) selected from the group consisting of transferrin receptor (TrfR), insulin-like growth factor type 1 receptor (IGFR), Erb-B2 Receptor Tyrosine Kinase 3 (ErbB3), leptin receptor (ObR), low-density lipoprotein receptor-related protein 1 (LRP-1), and receptor for advanced glycation end products (RAGE).

16. The modified cyclotide of any of claims 1-15, wherein the peptide is 10 to 25 amino acids.

17. The modified cyclotide of any of claims 1-16, wherein the BBB-R is expressed on brain endothelial cells.

18. The modified cyclotide of any of claims 1-17, wherein the peptide has blood-brain barrier translocation activity.

19. The modified cyclotide of any of claims 1-18, wherein the BBB-R is the transferrin receptor (TrfR).

20. The modified cyclotide of any of claims 1-19, wherein the peptide comprises the sequence set forth in any one of SEQ ID NOS: 26-34 and 49-54.

21. The modified cyclotide of any of claims 1-20, wherein the peptide has the consensus motif set forth as xxxxxHxxSWGx (SEQ ID NO:177).

22. The modified cyclotide of any of claims 1-20, wherein the modified cyclotide comprises the sequence set forth in any one of SEQ ID NOS: 72-80 and 95-100.

23. The modified cyclotide of any of claims 1-21, wherein the peptide comprises the sequence forth in SEQ ID NO:26.

24. The modified cyclotide of any of claims 1-23, wherein the modified cyclotide comprises the sequence set forth in SEQ ID NO:72.

25. The modified cyclotide of any of claims 1-21, wherein the peptide comprises the sequence forth in SEQ ID NO:49.

26. The modified cyclotide of any of claims 1-22 and 25, wherein the modified cyclotide comprises the sequence set forth in SEQ ID NO:95.

27. The modified cyclotide of any of claims 1-21, wherein the peptide comprises a sequence with 1, 2, 3 or 4 amino acid substitution(s) compared to the sequence set forth in of SEQ ID NOS: 26-34 and 49-54.

28. The modified cyclotide of claim 27, wherein the peptide comprises a sequence with 1, 2, 3 or 4 amino acid substitution(s) compared to the sequence set forth in of SEQ ID NO: 26.

29. The modified cyclotide of any of claims 1-28, wherein the amino acid substitution(s) is substitution of an amino acid to another amino acid selected from a histidine or an alanine.

30. The modified cyclotide of any of claims 1-21 and 27-29, wherein the peptide comprises the sequence set forth in any one of SEQ ID NOS: 55 and 117-128.

31. The modified cyclotide of any of claims 1-21 and 27-30, wherein the modified cyclotide comprises the sequence set forth in any one of SEQ ID NOS: 101 and 105-116.

32. The modified cyclotide of any of claims 1-21 and 27-29, wherein the peptide comprises the sequence set forth in SEQ ID NO:55.

33. The modified cyclotide of any of claims 1-21 and 27-32, wherein the modified cyclotide comprises the sequence set forth in any one of SEQ ID NOS: 101.

34. The modified cyclotide of any of claims 1-18, wherein the BBB-R is the leptin receptor.

35. The modified cyclotide of any of claims 1-18 and 34, wherein the peptide comprises the sequence set forth in any one of SEQ ID NOS: 10-23.

36. The modified cyclotide of any of claims 1-18 and 34-35, wherein the modified cyclotide comprises the sequence set forth in any one of SEQ ID NOS: 56-69.

37. The modified cyclotide of any of claims 1-18, wherein the BBB-R is ErbB3.

38. The modified cyclotide of any of claims 1-18 and 37, wherein the peptide comprises the sequence set forth in SEQ ID NO:24 or 25.

39. The modified cyclotide of any of claims 1-18, 37 and 38, wherein the modified cyclotide comprises the sequence set forth SEQ ID NO: 70 or 71.

40. The modified cyclotide of any of claims 1-18, wherein the BBB-R is insulin-like growth factor type 1 receptor (IGFR).

41. The modified cyclotide of any of claims 1-18 and 40, wherein the peptide comprises the sequence set forth in SEQ ID NO:35 or 36.

42. The modified cyclotide of any of claims 1-18, 40 and 41, wherein the modified cyclotide comprises the sequence set forth SEQ ID NO: 81 or 82.

43. The modified cyclotide of any of claims 1-18, wherein the BBB-R is RAGE.

44. The modified cyclotide of any of claims 1-18 and 43, wherein the peptide comprises the sequence set forth in SEQ ID NO:37 or 38.

45. The modified cyclotide of any of claims 1-18, 43 and 44, wherein the modified cyclotide comprises the sequence set forth SEQ ID NO: 83 or 84.

46. The modified cyclotide of any of claims 1-18, wherein the BBB-R is the 1 lipoprotein receptor-related protein 1 (LRP-1).

47. The modified cyclotide of any of claims 1-18 and 46, wherein the peptide comprises the sequence set forth in any one of SEQ ID NOS: 39-48.

48. The modified cyclotide of any of claims 1-18, 46 and 47, wherein the peptide comprises the sequence set forth in SEQ ID NO:39.

49. The modified cyclotide of any of claims 1-18, 46 and 47, wherein the peptide comprises the sequence set forth in SEQ ID NO:43.

50. The modified cyclotide of any of claims 1-18, 46 and 47, wherein the peptide comprises the sequence set forth in SEQ ID NO: 47.

51. The modified cyclotide of any of claims 1-18 and 46-50, wherein the modified cyclotide comprises the sequence set forth in any one of SEQ ID NOS: 85-94.

52. The modified cyclotide of any of claims 1-18, 46-48 and 51, wherein the modified cyclotide comprises the sequence set forth in SEQ ID NO:85.

53. The modified cyclotide of any of claims 1-18, 46, 47, 49 and 51, wherein the modified cyclotide comprises the sequence set forth in SEQ ID NO:89.

54. The modified cyclotide of any of claims 1-18, 46, 47, 50 and 51, wherein the modified cyclotide comprises the sequence set forth in SEQ ID NO:93.

55. A peptide comprising the amino acid sequence set forth in any of SEQ ID NOs: 10-55 or 117-128, wherein the peptide is 6-50 amino acids in length and binds to a receptor involved in blood-brain barrier transcytosis (BBB-R).

56. The peptide of claim 55, wherein the peptide has blood-brain barrier translocation activity.

57. The peptide of claim 55 and 56, wherein the peptide is 10 to 25 amino acids.

58. A peptide consisting of the sequence set forth in any one of SEQ ID NOs: SEQ ID NOs: 10-55 or 117-128.

59. The peptide of claim 58, wherein the peptide has blood-brain barrier translocation activity.

60. The peptide of claim 58 or claim 59, wherein the peptide binds to a receptor involved in blood-brain barrier transcytosis.

61. A peptide set forth by the sequence of any of SEQ ID NOS: 26-34 and 49-55 and 117-128, wherein the peptide binds the transferrin receptor.

62. The peptide of any of claims 55-61, wherein the peptide is set forth in SEQ ID NO:26.

63. The peptide of any of claims 55-61, wherein the peptide is set forth in SEQ ID NO: 49

64. The peptide of any of claims 55-61, wherein the peptide is set forth in SEQ ID NO: 55.

65. A peptide set forth by the sequence of any of SEQ ID NOS: 10-23, wherein the peptide binds the leptin receptor.

66. A peptide set forth by the sequence of any of SEQ ID NOS: 24 or 25, wherein the peptide binds ErbR3.

67. A peptide set forth by the sequence of any of SEQ ID NOS: 35 or 36, wherein the peptide binds IGFR.

68. The peptide of any of claims 55-60 and 67, wherein the peptide is set forth in SEQ ID NO:35.

69. The peptide of any of claims 55-60 and 67, wherein the peptide is set forth in SEQ ID NO:36.

70. A peptide set forth by the sequence of SEQ ID NO: 37 or 38, wherein the peptide binds the RAGE.

71. A peptide set forth by the sequence of any of SEQ ID NOS: 39-48, wherein the peptide binds the lipoprotein receptor-related protein 1 (LRP-1).

72. The peptide of any of claims 55-60 and 71, wherein the peptide is set forth in SEQ ID NO:39

73. The peptide of any of claims 55-60 and 71, wherein the peptide is set forth in SEQ ID NO: 47.

74. The peptide of any of claims 55-60 and 71, wherein the peptide is set forth in SEQ ID NO: 43.

75. The peptide of any of claims 55-74, wherein the peptide is synthetic.

76. A binding molecule comprising a binding scaffold and the peptide of any of claims 55-75.

77. The binding molecule of claim 76, wherein the binding scaffold is a cysteine-knot protein.

78. The binding molecule of claim 76 or claim 77 wherein the binding scaffold is a cyclotide and the peptide is inserted into or replaces one or more amino acids of a loop of a cyclotide backbone.

79. A nucleic acid molecule encoding the modified cyclotide of any of claims 1-54 or the binding molecule of any of claims 76-78.

80. A vector comprising the nucleic acid molecule of claim 79.

81. The vector of claim 80 that is an expression vector.

82. A host cell comprising the nucleic acid molecule of claim 79 or the vector of claim 80 or claim 81.

83. A method of producing a modified cyclotide or binding molecule, the method comprising introducing the nucleic acid of claim 79 or the vector of claim 80 or claim 81 into a host cell and culturing the host cell under conditions to express the protein in the cell, and optionally purifying the protein from the cell.

84. A purified modified cyclotide or binding molecule produced by the method of claim 83.

85. A conjugate comprising a modified cyclotide of any of claims 1-54 or binding molecule of any of claims 76-78, and a biologically active agent.

86. The conjugate of claim 85, wherein the biologically active agent is a small molecule, a peptide or a protein.

87. The conjugate of claim 85 or claim 86, wherein the biologically active agent is a diagnostic agent or a therapeutic agent.

88. The conjugate of any of claims 85-87 that is a fusion protein comprising the modified cyclotide operably linked to a biologically active agent that is a protein or peptide.

89. The conjugate of any of claims 85-88, wherein the biologically active agent is an antibody.

90. The conjugate of claim 89, wherein the antibody is directed against an antigen selected from the group consisting of human epidermal growth factor receptor 2 (HER2), beta-secretase 1 (BACE1), amyloid beta (Abeta), epidermal growth factor receptor (EGFR), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), caspase 6 and TNF-alpha.

91. The conjugate of claim 90, wherein the antibody is trastuzumab, adalimumab or aducanumab.

92. The conjugate of any of claims 85-88, wherein the biologically active agent is a growth factor or a hormone.

93. The conjugate of claim 92, wherein the biologically active agent is a growth factor and the growth factor is nerve growth factor (NGF) or Granulocyte colony-stimulating factor (GCSF).

94. The conjugate of any of claims 85-88, wherein the biologically active agent is an enzyme.

95. The conjugate of claim 94, wherein the enzyme is a ceramide degrading enzyme, a lipase, a hydrolase type enzyme or a sulfatase.

96. The conjugate of claim 94 or claim 95, wherein the enzyme is a ceramide degrading enzyme and the ceramide degrading enzyme is glucocerebrosidase, galactocerebrosidase or alpha galactosidase.

97. The conjugate of any of claims 94-96, wherein the enzyme is a glucocerebrosidase that has a sequence of amino acids that is at least 95% identical to the sequence set forth in SEQ ID NO:144 or SEQ ID NO:145.

98. The conjugate of any of claims 94-97, wherein the enzyme is a glucocerebrosidase that has the sequence of amino acids set forth in SEQ ID NO:144 or SEQ ID NO:145.

99. The conjugate of claim 94 or claim 95, wherein the enzyme is a lipase or a hydrolase type enzyme and the enzyme is sphingomyelinase, cerliponase or alpha glucosidase.

100. The conjugate of any of claims 85-99, wherein the conjugate is monovalent for binding a BBB-R.

101. The conjugate of any of claims 85-99, wherein the conjugate is bivalent for binding a BBB-R.

102. The conjugate of any of claims 85-99, wherein the conjugate is bispecific for binding two different BBB-R and comprises at least two different modified cyclotides that each bind to a different BBB-R.

103. The conjugate of any of claims 85-102, wherein the modified cyclotide is linked to the biologically active agent via a linker.

104. The conjugate of claim 103, wherein the linker is at least 10 amino acids in length.

105. The conjugate of claim 103, wherein the linker is at least 15 amino acids in length.

106. The conjugate of any of claims 103-105, wherein the linker is 10 to 20 amino acids.

107. The conjugate of any of claims 103-106, wherein the linker is a flexible GS peptide linker comprising the sequence GGGGS (SEQ ID NO:148), (GGGGS)₂ (SEQ ID NO:154) or (GGGGS)₃ (SEQ ID NO:155).

108. The conjugate of any of claims 103-107, wherein the linker is set forth in SEQ ID NO:104.

109. The conjugate of any of claims 103-106, wherein the linker is a cleavable linker comprising an endosome-specific protease cleavage site.

110. The conjugate of claim 109, wherein the endosome-specific protease cleavage site is a cathepsin cleavage site.

111. The conjugate of claim 110, wherein the cathepsin cleavage site is a cathepsin B cleavage site.

112. The conjugate of any of claims 103-106 and 109-111, wherein the linker comprises the sequence set forth in SEQ ID NO:133.

113. A pharmaceutical composition comprising the conjugate of any of claims 85-112, and a pharmaceutical carrier.

114. A method for transporting a biologically active agent across a blood brain barrier of an individual, the method comprising administering the conjugate of any of claims 85-112 or the pharmaceutical composition of claim 113 to an individual in need thereof.

115. The method of claim 114, wherein the individual has a neurological disease.

116. A method for treating a patient having a neurological disease comprising administering the conjugate of any of claims 85-112 or the pharmaceutical composition of claim 113 to said patient.

117. A method for diagnosing a neurological disease in a patient in need thereof comprising administering the conjugate of any of claims 85-112 or the pharmaceutical composition of claim 113 to said patient and wherein said conjugate comprises a radiolabel or detectable/diagnostic agent.

118. The pharmaceutical composition of claim 113 for use in the treatment of a neurological disease.

119. The pharmaceutical composition of claim 113 for use in the diagnosis of a neurological disease.

120. Use of the pharmaceutical composition of claim 113 in the manufacture of a medicament for use in the treatment of a neurological disease.

121. Use of the pharmaceutical composition of claim 113 in the manufacture of a medicament for use in the diagnosis of a neurological disease.


SEQUENCE LISTING (189 sequences)

SEQ ID NO: 1 - protein, 34 aa, synthetic construct
  REGION 1..34: note = MCOTI I
GGVCPKILQRCRRDSDCPGACICRGNGYCGSGSD

SEQ ID NO: 2 - protein, 34 aa, synthetic construct
  REGION 1..34: note = MCOTI II
GGVCPKILKKCRRDSDCPGACICRGNGYCGSGSD

SEQ ID NO: 3 - protein, 30 aa, synthetic construct
  REGION 1..30: note = MCOTI III
ERACPRILKKCRRDSDCPGACICRGNGYCG

SEQ ID NO: 4 - DNA, 102 bp, synthetic construct
  misc_feature 1..102: note = MCOTI II
  source 1..102: mol_type = other DNA
ggtggtgtttgtcctaaaattctcaaaaaatgtcgtcgtgattctgattgtcctggagcgtgtatttgtcgtggtaatggttattgcggttctggttctgat

SEQ ID NO: 5 - DNA, 68 bp, synthetic construct
  misc_feature 1..68: note = LOOP1FOR10
  source 1..68: mol_type = other DNA
  misc_difference 19..48: note = n = A,T/U,C,G or unknown or other
atggccggtggtgtttgtnnnnnnnnnnnnnnnnnnnnnnnnnnnnnntgtcgtcgtgattctgattg

SEQ ID NO: 6 - DNA, 74 bp, synthetic construct
  misc_feature 1..74: note = LOOP1FOR12
  source 1..74: mol_type = other DNA
  misc_difference 19..54: note = n = A,T/U,C,G or unknown or other
atggccggtggtgtttgtnnnnnnnnnnnnnnnnnnnnnnnnnnnnnnnnnnnntgtcgtcgtgattctgattg

SEQ ID NO: 7 - DNA, 25 bp, synthetic construct
  misc_feature 1..25: note = pIIISEQREV
  source 1..25: mol_type = other DNA
caactttcaacagtttcagcagagg

SEQ ID NO: 8 - DNA, 29 bp, synthetic construct
  misc_feature 1..29: note = LOOP1LIBPT
  source 1..29: mol_type = other DNA
gcccagccggccatggccggtggtgtttg

SEQ ID NO: 9 - DNA, 35 bp, synthetic construct
  misc_feature 1..35: note = LOOP5LIBPTREV
  source 1..35: mol_type = other DNA
tttctcctatgcggccgcatcagaaccagaaccgc

SEQ ID NO: 10 - protein, 12 aa, synthetic construct
  REGION 1..12: note = Hu ObR peptide
PWPKWTYMAQKY

SEQ ID NO: 11 - protein, 12 aa, synthetic construct
  REGION 1..12: note = Hu ObR peptide
QSFFWWHSWFGV

SEQ ID NO: 12 - protein, 12 aa, synthetic construct
  REGION 1..12: note = Hu ObR peptide
HSQFWPWEWYQF

SEQ ID NO: 13 - protein, 15 aa, synthetic construct
  REGION 1..15: note = Hu ObR peptide
WRWTPKIWVLEEMSS

SEQ ID NO: 14 - protein, 12 aa, synthetic construct
  REGION 1..12: note = Hu ObR peptide
WQGMMLFFVGEK

SEQ ID NO: 15 - protein, 12 aa, synthetic construct
  REGION 1..12: note = Hu ObR peptide
GWFYWYHKWFGP

SEQ ID NO: 16 - protein, 12 aa, synthetic construct
  REGION 1..12: note = Hu ObR peptide
YDWGWFWPFEQY

SEQ ID NO: 17 - protein, 12 aa, synthetic construct
  REGION 1..12: note = Hu ObR peptide
SDRWDWMFATWL

SEQ ID NO: 18 - protein, 12 aa, synthetic construct
  REGION 1..12: note = Hu ObR peptide
VQPWYSWFMEQG

SEQ ID NO: 19 - protein, 12 aa, synthetic construct
  REGION 1..12: note = Hu ObR peptide
PTQTWWFWLPAQ

SEQ ID NO: 20 - protein, 12 aa, synthetic construct
  REGION 1..12: note = Hu ObR peptide
YVDWPWTLTLWY

SEQ ID NO: 21 - protein, 12 aa, synthetic construct
  REGION 1..12: note = Hu ObR peptide
EWSDWFPYEFLF

SEQ ID NO: 22 - protein, 12 aa, synthetic construct
  REGION 1..12: note = Hu ObR peptide
PTAMWFDWFTVY

SEQ ID NO: 23 - protein, 12 aa, synthetic construct
  REGION 1..12: note = Hu ObR peptide
FLWFWYGFSRTL

SEQ ID NO: 24 - protein, 10 aa, synthetic construct
  REGION 1..10: note = Hu ErbB3 peptide
RHSYSQIPLW

SEQ ID NO: 25 - protein, 12 aa, synthetic construct
  REGION 1..12: note = Hu ErbB3 peptide
WWDKWVFPISRQ

SEQ ID NO: 26 - protein, 12 aa, synthetic construct
  REGION 1..12: note = HuTrfR peptide
LAHKWHLQSWGT

SEQ ID NO: 27 - protein, 10 aa, synthetic construct
  REGION 1..10: note = HuTrfR peptide
DPRWLAGWGS

SEQ ID NO: 28 - protein, 12 aa, synthetic construct
  REGION 1..12: note = MuTrfR peptide
TVWPFVWWGGVP

SEQ ID NO: 29 - protein, 12 aa, synthetic construct
  REGION 1..12: note = MuTrfR peptide
AYVHDWYHHVMY

SEQ ID NO: 30 - protein, 12 aa, synthetic construct
  REGION 1..12: note = MuTrfR peptide
MHNSASHNIQMQ

SEQ ID NO: 31 - protein, 12 aa, synthetic construct
  REGION 1..12: note = MuTrfR peptide
QTVFPMWPWYQL

SEQ ID NO: 32 - protein, 12 aa, synthetic construct
  REGION 1..12: note = MuTrfR peptide
HNLFVIESILFW

SEQ ID NO: 33 - protein, 10 aa, synthetic construct
  REGION 1..10: note = HuTrfR peptide
KALWYMGWGT

SEQ ID NO: 34 - protein, 10 aa, synthetic construct
  REGION 1..10: note = HuTrfR peptide
KQNWLPGWGT

SEQ ID NO: 35 - protein, 12 aa, synthetic construct
  REGION 1..12: note = HuIGFR peptide
FDPFYAMLQQLV

SEQ ID NO: 36 - protein, 12 aa, synthetic construct
  REGION 1..12: note = HuIGFR peptide
QGFYSWFAVQTM

SEQ ID NO: 37 - protein, 12 aa, synthetic construct
  REGION 1..12: note = Hu RAGE peptide
HVGYWWHIWMGA

SEQ ID NO: 38 - protein, 12 aa, synthetic construct
  REGION 1..12: note = Hu RAGE peptide
VSNFWFMHWFGV

SEQ ID NO: 39 - protein, 17 aa, synthetic construct
  REGION 1..17: note = Hu LRP-1 peptide
WLTLGIANKGIDVYDVK

SEQ ID NO: 40 - protein, 16 aa, synthetic construct
  REGION 1..16: note = Hu LRP-1 peptide
NPLKLKYWREWTYGGG

SEQ ID NO: 41 - protein, 15 aa, synthetic construct
  REGION 1..15: note = Hu LRP-1 peptide
YTQNRYKWLYAQTLN

SEQ ID NO: 42 - protein, 15 aa, synthetic construct
  REGION 1..15: note = Hu LRP-1 peptide
IEDRKHHRNLHQTVQ

SEQ ID NO: 43 - protein, 14 aa, synthetic construct
  REGION 1..14: note = Hu LRP-1 peptide
WYETKTKLYVGVVK

SEQ ID NO: 44 - protein, 12 aa, synthetic construct
  REGION 1..12: note = Hu LRP-1 peptide
LALTKPYWQWAM

SEQ ID NO: 45 - protein, 12 aa, synthetic construct
  REGION 1..12: note = Hu LRP-1 peptide
WHHEWITWWFWH

SEQ ID NO: 46 - protein, 12 aa, synthetic construct
  REGION 1..12: note = Hu LRP-1 peptide
DQSPYKLKHYGW

SEQ ID NO: 47 - protein, 12 aa, synthetic construct
  REGION 1..12: note = Hu LRP-1 peptide
PWYKMWFYETHR

SEQ ID NO: 48 - protein, 12 aa, synthetic construct
  REGION 1..12: note = Hu LRP-1 peptide
TPWKSQPLFWFQ

SEQ ID NO: 49 - protein, 12 aa, synthetic construct
  REGION 1..12: note = Hu TrfR peptide
EYFRWHLDSWGE

SEQ ID NO: 50 - protein, 12 aa, synthetic construct
  REGION 1..12: note = Hu TrfR peptide
LVSQWHIESWGQ

SEQ ID NO: 51 - protein, 12 aa, synthetic construct
  REGION 1..12: note = Hu TrfR peptide
QEERWHMHSWGP

SEQ ID NO: 52 - protein, 12 aa, synthetic construct
  REGION 1..12: note = Hu TrfR peptide
RYQEWHMESWGS

SEQ ID NO: 53 - protein, 12 aa, synthetic construct
  REGION 1..12: note = Hu TrfR peptide
LYEAWHLPSWGQ

SEQ ID NO: 54 - protein, 12 aa, synthetic construct
  REGION 1..12: note = Hu TrfR peptide
QMVSEHWVARVW

SEQ ID NO: 55 - protein, 12 aa, synthetic construct
  REGION 1..12: note = Hu TrfR peptide
LAHKWHLQSWHT

SEQ ID NO: 56 - protein, 40 aa, synthetic construct
  REGION 1..40: note = CYC1, Hu ObR
GGVCPWPKWTYMAQKYCRRDSDCPGACICRGNGYCGSGSD

SEQ ID NO: 57 - protein, 40 aa, synthetic construct
  REGION 1..40: note = CYC2, Hu ObR
GGVCQSFFWWHSWFGVCRRDSDCPGACICRGNGYCGSGSD

SEQ ID NO: 58 - protein, 40 aa, synthetic construct
  REGION 1..40: note = CYC3, Hu ObR
GGVCHSQFWPWEWYQFCRRDSDCPGACICRGNGYCGSGSD

SEQ ID NO: 59 - protein, 40 aa, synthetic construct
  REGION 1..40: note = CYC4, Hu ObR
GGVCWRWTPKIWVLEEMSSCSDCPGACICRGNGYCGSGSD

SEQ ID NO: 60 - protein, 40 aa, synthetic construct
  REGION 1..40: note = CYC5, Hu ObR
GGVCWQGMMLFFVGEKCRRDSDCPGACICRGNGYCGSGSD

SEQ ID NO: 61 - protein, 40 aa, synthetic construct
  REGION 1..40: note = CYC6, Hu ObR
GGVCGWFYWYHKWFGPCRRDSDCPGACICRGNGYCGSGSD

SEQ ID NO: 62 - protein, 40 aa, synthetic construct
  REGION 1..40: note = CYC7, Hu ObR
GGVCYDWGWFWPFEQYCRRDSDCPGACICRGNGYCGSGSD

SEQ ID NO: 63 - protein, 40 aa, synthetic construct
  REGION 1..40: note = CYC8, Hu ObR
GGVCSDRWDWMFATWLCRRDSDCPGACICRGNGYCGSGSD

SEQ ID NO: 64 - protein, 40 aa, synthetic construct
  REGION 1..40: note = CYC9, Hu ObR
GGVCVQPWYSWFMEQGCRRDSDCPGACICRGNGYCGSGSD

SEQ ID NO: 65 - protein, 40 aa, synthetic construct
  REGION 1..40: note = CYC10, Hu ObR
GGVCPTQTWWFWLPAQCRRDSDCPGACICRGNGYCGSGSD

SEQ ID NO: 66 - protein, 40 aa, synthetic construct
  REGION 1..40: note = CYC11, Hu ObR
GGVCYVDWPWTLTLWYCRRDSDCPGACICRGNGYCGSGSD

SEQ ID NO: 67 - protein, 40 aa, synthetic construct
  REGION 1..40: note = CYC12, Hu ObR
GGVCEWSDWFPYEFLFCRRDSDCPGACICRGNGYCGSGSD

SEQ ID NO: 68 - protein, 40 aa, synthetic construct
  REGION 1..40: note = CYC13, Hu ObR
GGVCPTAMWFDWFTVYCRRDSDCPGACICRGNGYCGSGSD

SEQ ID NO: 69 - protein, 40 aa, synthetic construct
  REGION 1..40: note = CYC14, Hu ObR
GGVCFLWFWYGFSRTLCRRDSDCPGACICRGNGYCGSGSD

SEQ ID NO: 70 - protein, 38 aa, synthetic construct
  REGION 1..38: note = CYC15, Hu ErbB3
GGVCRHSYSQIPLWCRRDSDCPGACICRGNGYCGSGSD

SEQ ID NO: 71 - protein, 40 aa, synthetic construct
  REGION 1..40: note = CYC16, Hu ErbB3
GGVCWWDKWVFPISRQCRRDSDCPGACICRGNGYCGSGSD

SEQ ID NO: 72 - protein, 40 aa, synthetic construct
  REGION 1..40: note = CYC17, HuTrfR
GGVCLAHKWHLQSWGTCRRDSDCPGACICRGNGYCGSGSD

SEQ ID NO: 73 - protein, 38 aa, synthetic construct
  REGION 1..38: note = CYC18, HuTrfR
GGVCDPRWLAGWGSCRRDSDCPETCICRGNGYCASGYD

SEQ ID NO: 74 - protein, 40 aa, synthetic construct
  REGION 1..40: note = CYC19, MuTrfR
GGVCTVWPFVWWGGVPCRRDSDCPGACICRGNGYCGSGSD

SEQ ID NO: 75 - protein, 40 aa, synthetic construct
  REGION 1..40: note = CYC20, MuTrfR
GGVCAYVHDWYHHVMYCRRDSDCPGACICRGNGYCGSGSD

SEQ ID NO: 76 - protein, 40 aa, synthetic construct
  REGION 1..40: note = CYC21, MuTrfR
GGVCMHNSASHNIQMQCRRDSDCPGACICRGNGYCGSGSD

SEQ ID NO: 77 - protein, 40 aa, synthetic construct
  REGION 1..40: note = CYC22, MuTrfR
GGVCQTVFPMWPWYQLCRRDSDCPGACICRGNGYCGSGSD

SEQ ID NO: 78 - protein, 40 aa, synthetic construct
  REGION 1..40: note = CYC23, MuTrfR
GGVCHNLFVIESILFWCRRDSDCPGACICRGNGYCGSGSD

SEQ ID NO: 79 - protein, 38 aa, synthetic construct
  REGION 1..38: note = CYC24, HuTrfR
GGVCKALWYMGWGTCRRDSDCPGACICRGNGYCGSGSD

SEQ ID NO: 80 - protein, 38 aa, synthetic construct
  REGION 1..38: note = CYC25, HuTrfR
GGVCKQNWLPGWGTCRRDSDCPGACICRGNGYCGSGSD

SEQ ID NO: 81 - protein, 40 aa, synthetic construct
  REGION 1..40: note = CYC26, HuIGFR
GGVCFDPFYAMLQQLVCRRDSDCPGACICRGNGYCGSGSD

SEQ ID NO: 82 - protein, 40 aa, synthetic construct
  REGION 1..40: note = CYC27, HuIGFR
GGVCQGFYSWFAVQTMCRRDSDCPGACICRGNGYCGSGSD

SEQ ID NO: 83 - protein, 40 aa, synthetic construct
  REGION 1..40: note = CYC28, Hu RAGE
GGVCHVGYWWHIWMGACRRDSDCPGACICRGNGYCGSGSD

SEQ ID NO: 84 - protein, 40 aa, synthetic construct
  REGION 1..40: note = CYC29, Hu RAGE
GGVCVSNFWFMHWFGVCRRDSDCPGACICRGNGYCGSGSD

SEQ ID NO: 85 - protein, 45 aa, synthetic construct
  REGION 1..45: note = CYC30, Hu LRP-1
GGVCWLTLGIANKGIDVYDVKCRRDSDCPGACICRGNGYCGSGSD

SEQ ID NO: 86 - protein, 44 aa, synthetic construct
  REGION 1..44: note = CYC31, Hu LRP-1
GGVCNPLKLKYWREWTYGGGCRRDSDCPGACICRGNGYCGSGSD

SEQ ID NO: 87 - protein, 43 aa, synthetic construct
  REGION 1..43: note = CYC32, Hu LRP-1
GGVCYTQNRYKWLYAQTLNCRRDSDCPGACICRGNGYCGSGSD

SEQ ID NO: 88 - protein, 43 aa, synthetic construct
  REGION 1..43: note = CYC33, Hu LRP-1
GGVCIEDRKHHRNLHQTVQCRRDSDCPGACICRGNGYCGSGSD

SEQ ID NO: 89 - protein, 42 aa, synthetic construct
  REGION 1..42: note = CYC34, Hu LRP-1
GGVCWYETKTKLYVGVVKCRRDSDCPGACICRGNGYCGSGSD

SEQ ID NO: 90 - protein, 40 aa, synthetic construct
  REGION 1..40: note = CYC35, Hu LRP-1
GGVCLALTKPYWQWAMCRRDSDCPGACICRGNGYCGSGSD

SEQ ID NO: 91 - protein, 40 aa, synthetic construct
  REGION 1..40: note = CYC36, Hu LRP-1
GGVCWHHEWITWWFWHCRRDSDCPGACICRGNGYCGSGSD

SEQ ID NO: 92 - protein, 40 aa, synthetic construct
  REGION 1..40: note = CYC37, Hu LRP-1
GGVCDQSPYKLKHYGWCRRDSDCPGACICRGNGYCGSGSD

SEQ ID NO: 93 - protein, 40 aa, synthetic construct
  REGION 1..40: note = CYC38, Hu LRP-1
GGVCPWYKMWFYETHRCRRDSDCPGACICRGNGYCGSGSD

SEQ ID NO: 94 - protein, 40 aa, synthetic construct
  REGION 1..40: note = CYC39, Hu LRP-1
GGVCTPWKSQPLFWFQCRRDSDCPGACICRGNGYCGSGSD

SEQ ID NO: 95 - protein, 40 aa, synthetic construct
  REGION 1..40: note = CYC40, Hu TrfR
GGVCEYFRWHLDSWGECRRDSDCPGACICRGNGYCGSGSD

SEQ ID NO: 96 - protein, 40 aa, synthetic construct
  REGION 1..40: note = CYC41, Hu TrfR
GGVCLVSQWHIESWGQCRRDSDCPGACICRGNGYCGSGSD

SEQ ID NO: 97 - protein, 40 aa, synthetic construct
  REGION 1..40: note = CYC42, Hu TrfR
GGVCQEERWHMHSWGPCRRDSDCPGACICRGNGYCGSGSD

SEQ ID NO: 98 - protein, 40 aa, synthetic construct
  REGION 1..40: note = CYC43, Hu TrfR
GGVCRYQEWHMESWGSCRRDSDCPGACICRGNGYCGSGSD

SEQ ID NO: 99 - protein, 40 aa, synthetic construct
  REGION 1..40: note = CYC44, Hu TrfR
GGVCLYEAWHLPSWGQCRRDSDCPGACICRGNGYCGSGSD

SEQ ID NO: 100 - protein, 40 aa, synthetic construct
  REGION 1..40: note = CYC45, Hu TrfR
GGVCQMVSEHWVARVWCRRDSDCPGACICRGNGYCGSGSD

SEQ ID NO: 101 - protein, 40 aa, synthetic construct
  REGION 1..40: note = CYC17-15H, Hu TrfR,
GGVCLAHKWHLQSWHTCRRDSDCPGACICRGNGYCGSGSD

SEQ ID NO: 102 - protein, 224 aa, synthetic construct
  REGION 1..224: note = Heavy chain (adalimumab)
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 103 - protein, 214 aa, synthetic construct
  REGION 1..214: note = Light chain (adalimumab)
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 16 aa, synthetic construct
  REGION 1..16: note = GS linker
GSGGGSGGGGSGGGGS

SEQ ID NO: 105 - protein, 40 aa, synthetic construct
  REGION 1..40: note = CYC17-5H, Hu TrfR
GGVCHAHKWHLQSWGTCRRDSDCPGACICRGNGYCGSGSD

SEQ ID NO: 106 - protein, 40 aa, synthetic construct
  REGION 1..40: note = CYC17-6H, Hu TrfR,
GGVCLHHKWHLQSWGTCRRDSDCPGACICRGNGYCGSGSD

SEQ ID NO: 107 - protein, 40 aa, synthetic construct
  REGION 1..40: note = CYC17-7A, Hu TrfR,
GGVCLAAKWHLQSWGTCRRDSDCPGACICRGNGYCGSGSD

SEQ ID NO: 108 - protein, 40 aa, synthetic construct
  REGION 1..40: note = CYC17-7A/10A, Hu TrfR,
GGVCLAAKWALQSWGTCRRDSDCPGACICRGNGYCGSGSD

SEQ ID NO: 109 - protein, 40 aa, synthetic construct; identical to SEQ ID NO: 72
  REGION 1..40: note = CYC17-8H, Hu TrfR,

SEQ ID NO: 110 - protein, 40 aa, synthetic construct
  REGION 1..40: note = CYC17-9H, Hu TrfR,
GGVCLAHKHHLQSWGTCRRDSDCPGACICRGNGYCGSGSD

SEQ ID NO: 111 - protein, 40 aa, synthetic construct
  REGION 1..40: note = CYC17-10A, Hu TrfR,
GGVCLAHKWALQSWGTCRRDSDCPGACICRGNGYCGSGSD

SEQ ID NO: 112 - protein, 40 aa, synthetic construct
  REGION 1..40: note = CYC17-11H, Hu TrfR,
GGVCLAHKWHHQSWGTCRRDSDCPGACICRGNGYCGSGSD

SEQ ID NO: 113 - protein, 40 aa, synthetic construct
  REGION 1..40: note = CYC17-12H, Hu TrfR,
GGVCLAHKWHLHSWGTCRRDSDCPGACICRGNGYCGSGSD

SEQ ID NO: 114 - protein, 40 aa, synthetic construct
  REGION 1..40: note = CYC17-13H, Hu TrfR,
GGVCLAHKWHLQHWGTCRRDSDCPGACICRGNGYCGSGSD

SEQ ID NO: 115 - protein, 40 aa, synthetic construct
  REGION 1..40: note = CYC17-14H, Hu TrfR,
GGVCLAHKWHLQSHGTCRRDSDCPGACICRGNGYCGSGSD

SEQ ID NO: 116 - protein, 40 aa, synthetic construct
  REGION 1..40: note = CYC17-16H, Hu TrfR,
GGVCLAHKWHLQSWGHCRRDSDCPGACICRGNGYCGSGSD

SEQ ID NO: 117 - protein, 12 aa, synthetic construct
  REGION 1..12: note = Hu TrfR peptide
HAHKWHLQSWGT

SEQ ID NO: 118 - protein, 12 aa, synthetic construct
  REGION 1..12: note = Hu TrfR peptide
LHHKWHLQSWGT

SEQ ID NO: 119 - protein, 12 aa, synthetic construct
  REGION 1..12: note = Hu TrfR peptide
LAAKWHLQSWGT

SEQ ID NO: 120 - protein, 12 aa, synthetic construct
  REGION 1..12: note = Hu TrfR peptide
LAAKWALQSWGT

SEQ ID NO: 121 - protein, 12 aa, synthetic construct
  REGION 1..12: note = Hu TrfR peptide
LAHKWHLQSWGT

SEQ ID NO: 122 - protein, 12 aa, synthetic construct
  REGION 1..12: note = Hu TrfR peptide
LAHKHHLQSWGT

SEQ ID NO: 123 - protein, 12 aa, synthetic construct
  REGION 1..12: note = Hu TrfR peptide
LAHKWALQSWGT

SEQ ID NO: 124 - protein, 12 aa, synthetic construct
  REGION 1..12: note = Hu TrfR peptide
LAHKWHHQSWGT

SEQ ID NO: 125 - protein, 12 aa, synthetic construct
  REGION 1..12: note = Hu TrfR peptide
LAHKWHLHSWGT

SEQ ID NO: 126 - protein, 12 aa, synthetic construct
  REGION 1..12: note = Hu TrfR peptide
LAHKWHLQHWGT

SEQ ID NO: 127 - protein, 12 aa, synthetic construct
  REGION 1..12: note = Hu TrfR peptide
LAHKWHLQSHGT

SEQ ID NO: 128 - protein, 12 aa, synthetic construct
  REGION 1..12: note = Hu TrfR peptide
LAHKWHLQSWGH

SEQ ID NO: 129 - protein, 453 aa, synthetic construct
  REGION 1..453: note = Heavy chain (aducanumab)
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 453 aa, synthetic construct
  REGION 1..453: note = Heavy chain -knob (aducanumab)
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 453 aa, synthetic construct
  REGION 1..453: note = Heavy chain -hole (aducanumab)
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 132 - protein, 214 aa, synthetic construct
  REGION 1..214: note = light chain (aducanumab)
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 14 aa, synthetic construct
  REGION 1..14: note = Cathepsin B (CB) cleavage site
LAASINHYAGYEQV

SEQ ID NO: 134 - protein, 449 aa, synthetic construct
  REGION 1..449: note = WT igH Trastuzumab
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 449 aa, synthetic construct
  REGION 1..449: note = KNOB igH Trastuzumab
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 136 - protein, 449 aa, synthetic construct
  REGION 1..449: note = HOLE igH Trastuzumab
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 137 - protein, 214 aa, synthetic construct
  REGION 1..214: note = Trastuzumab VL
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 506 aa, synthetic construct
  REGION 1..506: note = Trastuzumab-CYC17_IgH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGSGGGGSGGGGSLEGGVCLAHKWHLQSWGTCRRDSDCPGACICRGNGYCGSGSD

SEQ ID NO: 139 - protein, 506 aa, synthetic construct
  REGION 1..506: note = Trastuzumab KNOB CYC17_IgH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGGSGGGGSGGGGSLEGGVCLAHKWHLQSWGTCRRDSDCPGACICRGNGYCGSGSD

SEQ ID NO: 140 - protein, 520 aa, synthetic construct
  REGION 1..520: note = Trastuzumab KNOB CYC17 CB linker_IgH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGGSGGGGSGGGGSLELAASINHYAGYEQVGGVCLAHKWHLQSWGTCRRDSDCPGACICRGNGYCGSGSD

SEQ ID NO: 141 - protein, 506 aa, synthetic construct
  REGION 1..506: note = Trastuzumab KNOB CYC17-15H_IgH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGGSGGGGSGGGGSLEGGVCLAHKWHLQSWHTCRRDSDCPGACICRGNGYCGSGSD

SEQ ID NO: 142 - protein, 520 aa, synthetic construct
  REGION 1..520: note = Trastuzumab KNOB CYC17-15H-CB linker_IgH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGGSGGGGSGGGGSLELAASINHYAGYEQVGGVCLAHKWHLQSWHTCRRDSDCPGACICRGNGYCGSGSD

SEQ ID NO: 143 - protein, 520 aa, synthetic construct
  REGION 1..520: note = Trastuzumab HOLE CYC27-CBlinker_IgH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGSGGGSGGGGSGGGGSLELAASINHYAGYEQVGGVCQGFYSWFAVQTMCRRDSDCPGACICRGNGYCGSGSD

SEQ ID NO: 144 - protein, 497 aa, synthetic construct [sequence omitted]

SEQ ID NO: 145 - protein, 497 aa, synthetic construct
  REGION 1..497: note = Glucocerbrosidase
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 146 - protein, 555 aa, synthetic construct
  REGION 1..555: note = Glucocerebrosidase-CYC17 with linker
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQGSGGGSGGGGSGGGGSLEGGVCLAHKWHLQSWGTCRRDSDCPGACICRGNGYCGSGSD

SEQ ID NO: 147 - protein, 220 aa, synthetic construct
  REGION 1..220: note = NGF
PHSESNVPAGHTIPQVHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDSNPVDSGCRGIDSKHWNSYCTTTHTFVKALTMDGKQAAWEFIRIDTACVCVLSRKAVR

SEQ ID NO: 148 - protein, 5 aa, synthetic construct
  REGION 1..5: note = GS linker
GGGGS

SEQ ID NO: 149 - protein, 268 aa, synthetic construct
  REGION 1..268: note = NGF-CYC17 cyclotide fusion
PHSESNVPAGHTIPQVHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDSNPVDSGCRGIDSKHWNSYCTTTHTFVKALTMDGKQAAWEFIRIDTACVCVLSRKAVRGGGGSGNSGGVCLAHKWHLQSWGTCRRDSDCPGACICRGNGYCGSGSD

SEQ ID NO: 150 - protein, 14 aa, synthetic construct
  REGION 1..14: note = V5 epitope tag
GKPIPNPLLGLDST

SEQ ID NO: 151 - protein, 6 aa, synthetic construct
  REGION 1..6: note = His tag
HHHHHH

SEQ ID NO: 152 - protein, 20 aa, synthetic construct
  REGION 1..20: note = IL-2 secretory signal peptide
MYRMQLLSCIALSLALVTNS

SEQ ID NO: 153 - protein, 894 aa, synthetic construct
  REGION 1..894: note = CYC17-NGF-V5-His6 Trunc
CCAGGATCCACAGGCGAGCCTCACAGCGAGAGCAATGTGCCTGCCGGCCACACCATCCCTCAGGTGCACTGGACAAAGCTGCAGCACAGCCTGGACACCGCCCTGAGAAGGGCCAGATCTGCTCCTGCCGCCGCTATTGCCGCTAGAGTGGCCGGACAGACCCGGAACATCACCGTGGACCCCCGGCTGTTCAAGAAGCGGAGACTGAGAAGCCCCAGAGTGCTGTTCAGCACCCAGCCCCCTAGAGAGGCCGCCGATACCCAGGACCTGGATTTCGAAGTGGGCGGAGCCGCCCCTTTCAACCGGACCCACAGAAGCAAGCGGAGCAGCAGCCACCCCATCTTCCACAGAGGCGAGTTCAGCGTGTGCGACAGCGTGTCCGTGTGGGTGGGAGATAAGACCACCGCCACCGACATCAAGGGCAAAGAAGTGATGGTGCTGGGCGAAGTGAACATCAACAACAGCGTGTTCAAGCAGTACTTCTTCGAGACAAAGTGCCGGGACAGCAACCCCGTGGACAGCGGCTGTAGAGGCATCGACAGCAAGCACTGGAACAGCTACTGCACCACCACACACACCTTCGTGAAGGCCCTGACCATGGACGGAAAGCAGGCCGCCTGGGAGTTCATCAGAATCGACACCGCCTGCGTGTGCGTGCTGAGCAGAAAAGCTGTGCGGGGAGGCGGCGGAAGCGGGAATTCGGGTGGTGTTTGTCTGGCTCATAAATGGCATCTGCAGTCTTGGGGTACTTGTCGTCGTGATTCTGATTGTCCTGGAGCGTGTATTTGTCGTGGTAATGGTTATTGCGGTTCTGGTTCTGATGCGGCCGCAGGTAAACCTATCCCGAATCCGCTGCTGGGTCTGGACTCTACCCATCATCACCATCATCACTGA

SEQ ID NO: 154 - protein, 10 aa, synthetic construct
  REGION 1..10: note = (GGGGS)2 linker
GGGGSGGGGS

SEQ ID NO: 155 - protein, 15 aa, synthetic construct
  REGION 1..15: note = (GGGGS)3 linker
GGGGSGGGGSGGGGS

SEQ ID NO: 156 - protein, 10 aa, synthetic construct
  REGION 1..10: note = Cathepsin cleavable linker
QPMKRLTLGN

SEQ ID NO: 157 - protein, 10 aa, synthetic construct
  REGION 1..10: note = Cathepsin cleavable linker
GKPILFFRLK

SEQ ID NO: 158 - protein, 9 aa, synthetic construct
  REGION 1..9: note = Cathepsin cleavable linker
APISFFELG

SEQ ID NO: 159 - protein, 11 aa, synthetic construct
  REGION 1..11: note = Cathepsin cleavable linker
GRWPPMGLPWE

SEQ ID NO: 160 - protein, 11 aa, synthetic construct
  REGION 1..11: note = Cathepsin cleavable linker
GRWHPMGAPWE

SEQ ID NO: 161 - protein, 6 aa, synthetic construct
  REGION 1..6: note = Cathepsin cleavable linker
HPGGPQ

SEQ ID NO: 162 - protein, 24 aa, synthetic construct
  REGION 1..24: note = Cathepsin cleavable linker
TFLGGPKPPQRVMFTEDLKLPASF

SEQ ID NO: 163 - protein, 15 aa, synthetic construct
  REGION 1..15: note = Cathepsin cleavable linker
AVPAVTEGPIPEVLK

SEQ ID NO: 164 - protein, 20 aa, synthetic construct
  REGION 1..20: note = Cathepsin cleavable linker
LSQDTVSVPCQSASSASALG

SEQ ID NO: 165 - protein, 18 aa, synthetic construct
  REGION 1..18: note = Cathepsin cleavable linker
KGKVFQEPLFYEAPRSVD

SEQ ID NO: 166 - protein, 16 aa, synthetic construct
  REGION 1..16: note = Cathepsin cleavable linker
ITYKSNPNRILPDSVD

SEQ ID NO: 167 - protein, 14 aa, synthetic construct
  REGION 1..14: note = Cathepsin cleavable linker
MSYREAASGNFSLF

SEQ ID NO: 168 - protein, 14 aa, synthetic construct
  REGION 1..14: note = Cathepsin cleavable linker
NALKFLASLLELPE

SEQ ID NO: 169 - protein, 14 aa, synthetic construct
  REGION 1..14: note = Cathepsin cleavable linker
AGLTTELFSPVDLN

SEQ ID NO: 170 - protein, 14 aa, synthetic construct
  REGION 1..14: note = Cathepsin cleavable linker
MQYFSHFIRSGNPN

SEQ ID NO: 171 - protein, 14 aa, synthetic construct
  REGION 1..14: note = Cathepsin cleavable linker
AQTKLLAVSGPFHY

SEQ ID NO: 172 - protein, 14 aa, synthetic construct
  REGION 1..14: note = Cathepsin cleavable linker
YPYEFSRKVPTFAT

SEQ ID NO: 173 - protein, 14 aa, synthetic construct
  REGION 1..14: note = Cathepsin cleavable linker
TNSQLFRRAVLMGG

SEQ ID NO: 174 - protein, 14 aa, synthetic construct
  REGION 1..14: note = Cathepsin cleavable linker
KKKRKVGLEPGEKP

SEQ ID NO: 175 - protein, 10 aa, synthetic construct
  REGION 1..10: note = Cathepsin cleavable linker
KRKVGLEPGE

SEQ ID NO: 176 - protein, 8 aa, synthetic construct
  REGION 1..8: note = Cathepsin cleavable linker
RKVGLEPG

SEQ ID NO: 177 - protein, 12 aa, synthetic construct
  REGION 1..12: note = Transferrin receptor peptide consensus
  VARIANT 1..5: note = Xaa = Any Amino Acid
  VARIANT 7..8
  VARIANT 12
XXXXXHXXSWGX

SEQ ID NO: 178 - protein, 450 aa, synthetic construct
  REGION 1..450: note = HOLE igH Trastuzumab
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 179 - protein, 506 aa, synthetic construct
  REGION 1..506: note = Trastuzumab KNOB CYC26_IgH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGGSGGGGSGGGGSLEGGVCFDPFYAMLQQLVCRRDSDCPGACICRGNGYCGSGSD

SEQ ID NO: 180 - protein, 506 aa, synthetic construct
  REGION 1..506: note = Trastuzumab KNOB CYC27_IgH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGGSGGGGSGGGGSLEGGVCQGFYSWFAVQTMCRRDSDCPGACICRGNGYCGSGSD

SEQ ID NO: 181 - protein, 511 aa, synthetic construct
  REGION 1..511: note = Trastuzumab KNOB CYC30_IgH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGGSGGGGSGGGGSLEGGVCWLTLGIANKGIDVYDVKCRRDSDCPGACICRGNGYCGSGSD

SEQ ID NO: 182 - protein, 509 aa, synthetic construct
  REGION 1..509: note = Trastuzumab KNOB CYC32_IgH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGGSGGGGSGGGGSLEGGVCYTQNRYKWLYAQTLNCRRDSDCPGACICRGNGYCGSGSD

SEQ ID NO: 183 - protein, 508 aa, synthetic construct
  REGION 1..508: note = Trastuzumab KNOB CYC34_IgH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGGSGGGGSGGGGSLEGGVCWYETKTKLYVGVVKCRRDSDCPGACICRGNGYCGSGSD

SEQ ID NO: 184 - protein, 506 aa, synthetic construct
  REGION 1..506: note = Trastuzumab KNOB CYC28_IgH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGGSGGGGSGGGGSLEGGVCHVGYWWHIWMGACRRDSDCPGACICRGNGYCGSGSD

SEQ ID NO: 185 - protein, 506 aa, synthetic construct
  REGION 1..506: note = Trastuzumab KNOB CYC29_IgH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGGSGGGGSGGGGSLEGGVCVSNFWFMHWFGVCRRDSDCPGACICRGNGYCGSGSD

SEQ ID NO: 186 - protein, 12 aa, synthetic construct
  REGION 1..12: note = Linker
GGSGGSGGSGGS

SEQ ID NO: 187 - protein, 15 aa, synthetic construct
  REGION 1..15: note = Linker
GGSGGSGGSGGSGGS

SEQ ID NO: 188 - protein, 18 aa, synthetic construct
  REGION 1..18: note = Linker
GGGGGSGGGGGSGGGGGS

SEQ ID NO: 189 - protein, 18 aa, synthetic construct
  REGION 1..18: note = Linker
GGSGGGGSGGGGSGGGGS